(19) United States
(12) Patent Application Publication
(10) Pub. No.: US 2023/0365690 A1
(43) Pub. Date: Nov. 16, 2023

(54) METHODS OF TREATING MALIGNANT GLIOBLASTOMA

(71) Applicants: NORTHWESTERN UNIVERSITY, Evanston, IL (US); THE TRUSTEES OF COLUMBIA UNIVERSITY IN THE CITY OF NEW YORK, New York, NY (US)

(72) Inventors: Víctor Andrés Arrieta González, Chicago, IL (US); Raul Rabadan, New York, NY (US); Fabio M. Iwamoto, New York, NY (US); Adam M. Sonabend Worthalter, Chicago, IL (US)

(21) Appl. No.: 18/040,888

(22) PCT Filed: Aug. 8, 2021
(86) PCT No.: PCT/US2021/071138

§ 371 Date: Feb. 7, 2023

Related U.S. Application Data
(60) Provisional application No. 63/062,805, filed on Aug. 7, 2020.

(51) Int. Cl. C07K 16/28; A61P 35/04; A61K 45/06; G01N 33/574

(52) U.S. Cl. CPC C07K 16/2818; A61P 35/04; A61K 45/06; G01N 33/57484; G01N 2440/14; G01N 2333/91205

(57) ABSTRACT

Disclosed herein are methods for identifying subjects, diagnosed as having glioblastoma (GBM), as likely responders to treatment with a programmed death protein 1 (PD-1) blockade. In some embodiments, the subjects are identified by determining the level of phosphorylated extracellular-signal-regulated kinase (p-ERK) in a GBM tumor sample from the subject. In some embodiments, the GBM is recurrent. Also disclosed herein are methods of treating GBM subjects, whereby the methods include (1) identifying the subject as a likely responder to PD-1 blockade, and (2) administering a PD-1 inhibitor and/or a programmed death-ligand 1 (PD-L1) inhibitor.

### CROSS-REFERENCE TO RELATED APPLICATIONS

This application claims the benefit of U.S. Provisional Application No. 63/062,805 filed Aug. 7, 2020, the content of which is incorporated herein by reference in its entirety.

### STATEMENT REGARDING FEDERALLY SPONSORED RESEARCH OR DEVELOPMENT

This invention was made with government support under OD021356, OD021356-05, NS110703, and CA221747 awarded by the National Institutes of Health. The government has certain rights in the invention.

### BACKGROUND

The prognosis for patients with malignant gliomas is poor. For example, patients suffering from one type of malignant glioma, glioblastoma (also known as glioblastoma multiforme, “GBM”) is extremely poor with a median overall survival (OS) of around 21 months. Whereas radiation, chemotherapy, and tumor-treating fields are established treatments at the time of diagnosis, at recurrence, there are no established effective therapies. This is attributed partially to the notorious molecular and microenvironmental heterogeneity of glioma, of which GBM is an example, which contributes to erratic and unpredictable responses to specific therapies. Thereby, the variable response to therapy across glioma patients, such as GBM patients, often manifests as negative clinical trials in which an elusive subset of patients exhibits durable response.

Immune checkpoint blockade has seen an unparalleled expansion in cancer therapy leading to long-term remissions even in cases with advanced metastatic disease. It has been adopted as the standard of care of advanced melanoma, non-small cell lung cancer, clear-cell renal cell carcinoma, solid tumors with DNA mismatch repair deficiency or microsatellite instability (MSI), and an increasing number of other cancers. In contrast, its success has been limited in GBM, partly due to the diverse and iterative mechanisms of intrinsic and iatrogenic immunosuppression. These include tumor infiltration by immunosuppressive cells, defects in the antigen processing and presentation machinery, sequestration of T cells in the bone marrow, and frequent use of immunosuppressive medications such as corticosteroids, among other factors. Whereas several negative trials showed an overall lack of efficacy of immune checkpoint blockade for GBM patients, durable clinical responses have been reported in a subset of patients.

The present inventors recently reported an analysis of recurrent GBM patients treated with adjuvant PD-1 blockade, where they uncovered molecular determinants of immunotherapy response. Response was based on imaging and pathological criteria and responsive patients exhibited significant prolongation of survival independent of other therapies and known clinical or molecular prognostic variables.

BRAF and PTPN11 activating mutations, which are known to drive MAPK/ERK pathway signaling were enriched in recurrent GBM patients that responded to PD-1 blockade. However, somatic mutations on these MAPK pathway genes were encountered in only 30% of patients who responded to PD-1 inhibitors in our GBM cohort. In the unselected population from TCGA, only 2-3% of GBM subjects present such mutations. Thus, whereas these mutations might provide biological clues into the GBM biology associated to PD-1 inhibitors, these alterations have limited value as a predictive biomarker given that 70% of responder patients were not identified by these mutations. Accordingly, there is a need in the art to definitively identify GBM patients who will benefit from PD-1 therapies.

### SUMMARY

Disclosed herein are methods for identifying subjects, diagnosed as having malignant gliomas, e.g., glioblastoma (GBM), as likely responders to treatment with a programmed death protein 1 (PD-1) blockade. In some embodiments, the subjects are identified by determining the level of phosphorylated extracellular-signal-regulated kinase 1/2 (p-ERK) in a tumor sample, such as a GBM tumor sample, from the subject. In some embodiments, the GBM is recurrent. Also disclosed herein are methods of treating malignant gliomas, such as GBM in subjects, whereby the methods include (1) identifying the subject as a likely responder to PD-1 blockade, and (2) administering a PD-1 inhibitor. In some embodiments, additionally or alternatively, a programmed death-ligand 1 (PD-L1) inhibitor is administered.

### BRIEF DESCRIPTION OF THE DRAWINGS

FIG. 1a-b. ERK1/2 activation is a predictive biomarker of radiographic response to anti-PD-1 immunotherapy in recurrent GBM patients. a, Dot plot showing the quantification of p-ERK⁺ cells prior to PD-1 blockade initiation in responder and non-responder patients as previously defined¹⁴ (n=29). P=0.0029, two-sided Mann Whitney U test. Data is presented as mean±s.d. Each dot represents an independent patient sample. b, (Top) Example of a responder patient showing the corresponding Mill, the associated H&E staining and immunostaining for p-ERK in the pre-treatment sample. A biopsy performed in a gadolinium-enhancing lesion 10 months after treatment with the anti-PD-1 therapy showed few tumor cells and a profuse CD3⁺ T cell infiltrate. Flow cytometry analysis in the CSF and brain tumor showed that many of these T cells were CD4⁺ and CD8⁺. This patient experienced stable disease for at least 21 months after immunotherapy initiation. (Bottom) A non-responder patient with the corresponding Mill, H&E and p-ERK immunostaining in the pre-treatment sample. Arrows point to p-ERK⁺ endothelial cells.

FIG. 2a-d. ERK1/2 phosphorylation evaluated by semi-automatic IHC quantification shows that is a predictive biomarker following PD-1 blockade in recurrent GBM. a, Kaplan-Meier curve comparing OS of recurrent GBM patients defined as either high or low p-ERK by quantitative IHC measurement counting from initiation of PD-1 blockade (anti-PD-1 therapy group, n=29) and from surgery at recurrence (no-immunotherapy group, n=33); P values, two-sided log-rank test. b, Forest plots representing the univariable and multivariable survival analysis using a Cox proportional hazard model evaluating the MGMT promoter methylation status, IDH mutations, KPS, age, and p-ERK⁺ cell density on survival in the anti-PD-1 therapy cohort (top) and the No immunotherapy cohort (bottom) presented as Hazard ratios (95% CI). P values by two-sided Wald test. c, ROC curve of sensitivity and 1-specificity displaying the mean AUC (95% CI) for the anti-PD-1 therapy cohort and the No immunotherapy cohort. d, (left), Dot plot comparing the quantification of p-ERK⁺ cell density between tumors GBM patients harboring either BRAF/PTPN11 mutations (n=4), wild-type BRAF/PTPN11 (n=5), or unknown BRAF/PTPN11 status (n=2) that had an OS >12 months with those that had wild type (n=11) or unknown BRAF/PTPN11 status (n=7) and lived <12 months after initiation of the immunotherapy. (right) H&E and p-ERK immunostaining of three GBM samples. From top to bottom: a BRAF mutated tumor and a wild-type BRAF tumor from patients that lived more than 12 months; and a wild-type BRAF tumor from a patient that lived less than 12 months. P values by two-sided Mann Whitney U test. Data is presented as mean±s.d. Each dot represents an independent patient sample.

FIG. 3a-f. Validation of pre-treatment p-ERK staining correlates with OS following PD-1 blockade in an independent recurrent GBM cohort treated with adjuvant PD1 blockade in the context of a prospective clinical trial from Cloughesy T et al.¹⁶ a, Cartoon showing the timepoints of surgical tumor acquisition relative to treatment with PD-1 blockade identified as pre-study or on-study tumor samples. b, (left) Change in p-ERK cell density in pre-study and on-study tumor samples. Red and blue dots represent high and low p-ERK tumors, respectively. Dashed green line represent the optimized cut-point value used to partition high and low p-ERK tumors. P value calculated using two-sided Wilcoxon signed-rank test. Each dot represents an independent patient sample. (right) Representative IHC micrographs showing the change of p-ERK immunostaining between paired tumor samples. c, d, Kaplan-Meier plots showing OS in high vs low p-ERK groups treated with adjuvant PD-1 blockade evaluating pre-study tumor samples (c) and on-study tumor samples (d). e, Forest plots for on-study tumor samples representing the univariable and multivariable survival analysis using a Cox proportional hazard model evaluating potential covariates influencing survival presented as Hazard ratios (95% CI). P values by two-sided Log-rank test and two-sided Wald test. f, ROC curve of sensitivity and 1-specificity displaying the mean AUC (95% CI) for the validation GBM cohort.

FIG. 4a-f. Multiplex immunofluorescence staining of recurrent GBM samples shows p-ERK positivity in SOX2⁺ cells and associated myeloid cell infiltration. a, Bar plot showing the contribution to p-ERK expression from SOX2⁺, TMEM119⁺, CD163⁺, and other cells (SOX2⁻ TMEM119⁻ CD163⁻ cells). Differences among cell types were evaluated using one-way ANOVA with post hoc Tukey's multiple comparisons test (n=13) b, Dot plot showing the comparison of SOX2⁺ p-ERK⁺ cells/mm² between high and low p-ERK tumors (n=13). c, Representative images of three different tumor samples. From top to bottom: a BRAF^{V600E} GBM sample having high p-ERK staining, a wild-type BRAF/PTPN11 GBM having high p-ERK staining, and a wild-type BRAF/PTPN11 GBM displaying low p-ERK staining. For the three tumor samples: (left) H&E and p-ERK IHC images of the same tumor region. (middle), Multiplex immunofluorescence images showing the markers for SOX2, p-ERK, and DAPI. (right) Multiplex immunofluorescence images showing the markers for SOX2, TMEM119, CD163, and DAPI. Arrows point to SOX2⁺ p-ERK⁺ cells. d, Dot plot showing the comparison of TMEM119⁺ between high and low p-ERK tumors (n=13). e, Dot plot showing the comparison of CD163⁺ cells/mm² between high and low p-ERK tumors (n=13). f, (left) Scatter plot showing the correlation of p-ERK⁺ cells/mm² with Iba1⁺ cells/mm² obtained by software-based quantification of IHC stained tumor samples. (right) Representatives images of p-ERK and Iba1 immunostainings of the same tumor region (n=12). P value by Pearson's correlation. e, P value by two-sided Mann Whitney U test in b, d, and e. Data is presented as mean±s.d. in a, b, d, and e. Each dot represents an independent patient sample in a, b, d, e, and f a, Bar plot showing the contribution to p-ERK expression from SOX2⁺, TMEM119⁺, CD163⁺, and other cells (SOX2⁻ TMEM119⁻ CD163⁻ cells). Differences among cell types were evaluated using one-way ANOVA with post hoc Tukey's multiple comparisons test (n=13) b, Dot plot showing the comparison of SOX2⁺ p-ERK⁺ cells/mm² between high and low p-ERK tumors (n=13). c, Representative images of three different tumor samples. From top to bottom: a BRAF^{V600E} GBM sample having high p-ERK staining, a wild-type BRAF/PTPN11 GBM having high p-ERK staining, and a wild-type BRAF/PTPN11 GBM displaying low p-ERK staining. For the three tumor samples: (left) H&E and p-ERK IHC images of the same tumor region. (middle), Multiplex immunofluorescence images showing the markers for SOX2, p-ERK, and DAPI. (right) Multiplex immunofluorescence images showing the markers for SOX2, TMEM119, CD163, and DAPI. Arrows point to SOX2⁺ p-ERK⁺ cells. d, Dot plot showing the comparison of TMEM119⁺ between high and low p-ERK tumors (n=13). e, Dot plot showing the comparison of CD163+ cells/mm² between high and low p-ERK tumors (n=13). f, (left) Scatter plot showing the correlation of p-ERK⁺ cells/mm² with Iba1⁺ cells/mm² obtained by software-based quantification of IHC stained tumor samples. (right) Representatives images of p-ERK and Iba1 immunostainings of the same tumor region (n=12). P value by Pearson's correlation. e, P value by two-sided Mann Whitney U test in b, d, and e. Data is presented as mean±s.d. in a, b, d, and e. Each dot represents an independent patient sample in a, b, d, e, and f.

FIG. 5a-g. Spatial analysis of the tumor cells expressing p-ERK and their associated myeloid cells. a, Cartoon representing the calculation of distances from TMEM119⁺ and CD163⁺cells to SOX2⁺ p-ERK⁺/p-ERK⁻ cells. b, c, Bar plots comparing the mean distances from TMEM119⁺ (b) and CD163⁺ (c) cells to SOX2⁺ p-ERK⁺/p-ERK⁻ cells in high vs low p-ERK tumors. Dots represent tumor samples (n=13). d, Representative multiplex immunofluorescence images illustrating the spatial dimensions between TMEM119⁺ cells and SOX2⁺ p-ERK⁺ in a high and a low p-ERK GBM. e, Cartoon representing the calculation of distances from CD163+ cells to GFAP⁺ p-ERK⁺/p-ERK⁻ cells. f, Bar plots comparing the mean distances from CD163⁺ cells to GFAP⁺ p-ERK⁺/p-ERK⁻ cells in high vs low p-ERK tumors. Dots represent recurrent glioblastoma tumor samples (n=6). g Representative multiplex immunofluorescence images illustrating the spatial dimensions between CD163⁺ cells and GFAP⁺ p-ERK⁺ in a high and a low p-ERK GBM. P values by two-tailed unpaired t test in b, c, and f.

FIG. 6a-h. Single-cell RNA-seq of GBM patients from high and low p-ERK groups. a, Quantification of and representative p-ERK IHC images and multiplex immunofluorescence images of GBM samples that were used analysis. Dashed green line represent the cut-point value used to partition high and low p-ERK tumors in the discovery and validation cohorts. b, UMAP graph showing the expression of validated cell markers for tumor cells (SOX2), myeloid cells (CD14), endothelial cells (VWF), and pericytes (PDGFRB). The color key indicates the expression levels. Each dot represents an individual cell. c, UMAP graphs showing the overlapping annotations derived from high and low p-ERK IHC stained tumors obtained from the software-based quantification in all cells (top) and in the myeloid cell compartment (bottom). d, GO terms analysis. Differentially expressed gene signatures of the myeloid cell population infiltrating high and low p-ERK GBM samples. 27 differentially expressed GO terms are represented in a dot plot, with the size of the dot corresponding to the percentage of genes that matched the GO term. The color of the dots corresponds to the q value of enrichment. e, (top) UMAP plot showing the expression of the “MHC class II protein binding complex” gene signature in the myeloid cells. (bottom) GSEA plot showing enrichment of the GO term “MHC class II protein binding complex” within myeloid cells derived from high and low p-ERK tumors. f, g, Beeswarm plot showing the cell density of TMEM119⁺ MCHII⁺ cells (f) and CD163⁺ MHCII⁺ cells (g) between high and low p-ERK tumors (n=23). P value by two-sided Mann Whitney U test. h, Representative multiplex immunofluorescence images illustrating the expression of MHC II by TMEM119⁺ and CD163⁺ cells in a high and a low p-ERK GBM sample. SOX2, TMEM119, CD163, MHC II and DAPI are included as cell markers.

FIG. 7a-c. Optimization of the staining technique for p-ERK antibody. a, Titration of the p-ERK antibody (clone: D13.14.4E) using different dilutions performed in GBM samples. We show the same region of a GBM sample stained with the indicated dilutions of the p-ERK antibody with a low and high magnification image for each dilution. b, (left) Microarray containing breast cancer tissues stained with p-ERK antibody (1:500 dilution) representing a positive control. (right) Magnification of one the breast cancer tissues showing specific staining in the endothelium (red rectangle). c, (left) Non-tumoral brain tissue stained with p-ERK antibody (1:500 dilution) representing a negative control. (right) Magnification of the white matter showing p-ERK staining with minimal background.

FIG. 8. Workflow used for the software-based quantification of p-ERK⁺ cells. After a certified neuropathologist outlined the tumor regions in digitized whole-slide images for each case, several representative regions of interest (ROI) were selected to measure p-ERK⁺ cell density using quantitative image analysis software. Intensity staining parameters were adjusted to identify cells with p-ERK⁺ staining in segmented nuclei and their surrounding cytoplasmic regions. The same software parameters were used to analyze all cases in the discovery, validation, and scRNA-seq cohorts.

FIG. 9. Gating strategy used for flow cytometry analysis. By employing an SSC-A vs FCS-A density plot, we gated lymphocytes based on their known location with these parameters. Then, cells were gated for singlets using an FSC-H vs FSC-A plot. The lymphocyte/singlet gate was further analyzed to identify live/dead cells. Next, CD45⁺ cells were identified. Finally, CD4⁺ cells in the X axis and CD8⁺ in the Y axis were plotted.

FIG. 10. Quantification of p-ERK⁺ cell density in tumoral regions. (left) Dot plot showing the distribution of p-ERK quantification of all GBM samples treated and non-treated with PD-1 blockade (n=62). (right) From top to bottom, micrographs showing one high p-ERK tumor sample and two low p-ERK tumor samples with positive staining in the endothelial cells (red arrows). In the dot plot, the magenta dot represents CU100 patient, the green dot represents NU01688 patient, and the red dot represents CU110 patient.

FIG. 11a-c. ERK1/2 phosphorylation is a predictive biomarker of OS following PD-1 blockade in recurrent GBM. a, Conditional inference trees analysis for cut-point optimization in the GBM cohort treated with PD-1 blockade reveals a cut-point value similar to the median of all tumor samples. b, Forest plot representing the univariable analysis using a Cox regression model evaluating the clinical and molecular prognostic factors that might confound the association between survival p-ERK and presented as Hazard ratio (95% CI). P value by two-sided Wald test. c, Kaplan-Meier curve comparing OS of recurrent GBM patients scored as either high or low p-ERK by assessment of a neuropathologist counting from initiation of PD-1 blockade (anti-PD-1 therapy group, n=29) and from surgery at recurrence (no-immunotherapy group, n=33). p-ERK scores in tumor regions were designated as follows: 0-1 were considered as low, and 2-3 as high; P value by two-sided log-rank test.

FIG. 12. Peptide competition assay neutralizing the p-ERK1/2 antibody tested in GBM samples. We obtained tissue sections and extracted protein from a set of FFPE GBM samples from our cohort. In parallel, we neutralized the p-ERK1/2 antibody with a specific blocking peptide. Then, we performed western blot in which we loaded the extracted proteins from the GBM samples. One western blot was incubated with the neutralized p-ERK antibody and the other with the free p-ERK antibody. In addition, we performed IHC in the same GBM samples used to perform western blot employing the neutralized and free p-ERK antibody to perform the staining.

FIG. 13. Preservation of the p-ERK epitope in FPPE GBM tissues. Western blot targeting p-ERK, ERK1, ERK2, p-AKT, AKT, and β-actin in a subset of GBM samples used for survival analysis.

FIG. 14a-c. Evaluation of the impact of ischemic time on p-ERK degradation by IHC and western blot, and comparison to the samples used in our study. a, Representatives images of the analysis conducted to evaluate p-ERK degradation in endothelial cells of GBM samples at different periods of ischemic time. For this, 3 human tumor specimens were obtained during surgery, and immediately divided into similar size portions, which then were subjected to different ischemic times before processing. Specific endothelial cells subjected to analysis are labeled with colors assigned by the software. b, Bar plots and dot plots showing p-ERK⁺ cell/mm² in tumor regions, p-ERK intensity of endothelial cells within samples used to evaluate the effect of ischemic time on p-ERK degradation, and tumor samples used for survival analysis (PD-1 immunotherapy cohort and no immunotherapy cohort). Each dot represents one ROI analyzing one endothelial cell. Green dots represent a statistically significant group compared to the group of 0 hrs. of ischemic time represented as gray dots. (All samples were normalized to the average of values of the three 0 hrs. groups). P values by Kruskal Wallis test with post hoc Dunn's multiple comparison test. c, Western blot showing p-ERK and other phosphoproteins in samples subjected to different ischemic times. Densitometry analysis for p-ERK western blot was performed using ERK1 and ERK2 staining. For this densitometry, every patient had density normalized by 0 minutes of ischemic time. Error bars represent SEM.

FIG. 15a-b. Progression-free survival of the validation cohort from Cloughesy T et al.¹⁶ clinical trial. a, b, Kaplan-Meier showing progression-free survival following PD-1 blockade based on p-ERK high vs low for pre-study (a) and on-study (b) tumor samples.

FIG. 16a-b. Multiplex immunofluorescence staining of recurrent GBM samples employing GFAP marker. a, Bar plot showing the comparison of GFAP⁺ p-ERK⁺ cells and other cells expressing p-ERK⁺. P value by two-sided Mann Whitney U test. b, Representative images of three different tumor samples. From top to bottom: a BRAF^{V600E} GBM sample having high p-ERK staining, a wild-type BRAF/PTPN11 GBM having high p-ERK staining, and a wild-type BRAF/PTPN11 GBM displaying low p-ERK staining. For the three tumor samples: (left) H&E and p-ERK IHC images of the same tumor region. (middle), Multiplex immunofluorescence images showing the markers for GFAP, p-ERK, and DAPI. (right) Multiplex immunofluorescence images showing the markers for GFAP, CD163, and DAPI.

FIG. 17. Single-cell RNA seq of GBM patients with high and low p-ERK IHC staining. UMAP representation of 21,326 individual cells from 10 GBM patients measured with scRNA-seq (left). UMAP graph showing the representation of 1758 myeloid cells derived from the 10 GBM patients (right). Each dot represents an individual cell.

FIG. 18. Table 1 showing demographics and clinical data of GBM patients included in the discovery cohort.

FIG. 19. Shows plots of Schoenfeld residuals in the discovery cohort (left panel) and the validation cohort (right panel).

FIG. 20a-b. a shows a table summarizing clinical characteristics of the GBM cohort rerated with anti-PD-1 therapy. Col. 1 Patient ID; Col. 2 Age; Col. 3 Gender; Col. 4 IHD status; Col. 5 MGMT methylation; Col. 6 KPS; Col. 7 Concurrent treatments; Col. 8 Survival days; Col. 9 Survival status (1=dead; 0=alive); Col. 10 Tumor size; Col. 11Ki67; Col 12 steroid dose (mg/kg); Col. 13 Days from surgery to immunotherapy; Col. 14 pERK cells/mm² quantification; Col. 15 pERK score; Col. 16 Endothelial pERK staining. b shows a table summarizing clinical characteristics of the GBM cohort that did not receive immunotherapy. Col. 1 Patient ID; Col. 2 Age; Col. 3 Gender; Col. 4 IDH status; Col. 5 MGMT methylation; Col. 6 KPS; Col. 7 Survival days; Col. 8 Survival status (1=dead; 0=alive); Col. 9 pERK cell/mm² quantification; Col.10 pERK score; Col. 11 Endothelial pERK staining.

FIG. 21. Is a Table showing clinical characteristics of the validation cohort treated with adjuvant PD-1 blockade. Col. 1 Patient ID; Col. 2 Gender; Col. 3 Age; Col. 4 OS Status; Col. 5 OS days; Col. 6 PFS status; Col. 7 PFS days; Col. 8 MGMT; Col. 9 IDH status; Col. 10 KPS; Col. 11 Steroid dose; Col. 12 Percent resected; Col. 13 days from surgery to treatment; Col. 14 pERK quantification on study samples; Col. 15 p-ERK quantification pre study samples.

FIG. 22. Is a Table showing results of an investigation of the transcriptional differences within the myeloid cell population between high and low p-ERK tumors, using gene set enrichment analysis (GSEA) with the gene ontology (GO) set collection. With this analysis, 27 pathways were significantly enriched in the myeloid cells derived from high p-ERK tumors. Co; 1 Go Term; Col. 2 es; Col. 3 nes; Col. 4 pval; Col. 5 fdr; Col. 6 geneset_size; Col. 7 matched_size; Col. 8 genes.

### DETAILED DESCRIPTION

The present invention is described herein using several definitions, as set forth below and throughout the application.

### Definitions

Unless otherwise specified or indicated by context, the terms “a”, “an”, and “the” mean “one or more.” For example, “an inhibitor of tumor cell aggregation” should be interpreted to mean “one or more inhibitors of tumor cell aggregation.”

As used herein, “about,” “approximately,” “substantially,” and “significantly” will be understood by persons of ordinary skill in the art and will vary to some extent on the context in which they are used. If there are uses of these terms which are not clear to persons of ordinary skill in the art given the context in which they are used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising” in that these latter terms are “open” transitional terms that do not limit claims only to the recited elements succeeding these transitional terms. The term “consisting of,” while encompassed by the term “comprising,” should be interpreted as a “closed” transitional term that limits claims only to the recited elements succeeding this transitional term. The term “consisting essentially of” while encompassed by the term “comprising,” should be interpreted as a “partially closed” transitional term which permits additional elements succeeding this transitional term, but only if those additional elements do not materially affect the basic and novel characteristics of the claim.

As used herein, the term “subject” may be used interchangeably with the term “patient” or “individual” and may include an “animal” and in particular a “mammal.” Mammalian subjects may include humans and other primates, domestic animals, farm animals, and companion animals such as dogs, cats, guinea pigs, rabbits, rats, mice, horses, cattle, cows, and the like.

In some embodiments, a subject may be in need of treatment, for example, treatment may include administering a therapeutic amount of one or more agents that inhibits the programmed cell death 1 (PD-1) pathway (e.g., a PD-1 and/or a PD-L1 inhibitor).

As used herein, the phrase “effective amount” shall mean that drug dosage that provides the specific pharmacological response for which the drug is administered in a significant number of patients in need of such treatment. An effective amount of a drug that is administered to a particular patient in a particular instance will not always be effective in treating the conditions/diseases described herein, even though such dosage is deemed to be a therapeutically effective amount by those of skill in the art.

As used herein, the terms “treat” or “treatment” encompass both “preventative” and “curative” treatment. “Preventative” treatment is meant to indicate a postponement of development of a disease, a symptom of a disease, or medical condition, suppressing symptoms that may appear, or reducing the risk of developing or recurrence of a disease or symptom. “Curative” treatment includes reducing the severity of or suppressing the worsening of an existing disease, symptom, or condition. Thus, treatment includes ameliorating or preventing the worsening of existing disease symptoms, preventing additional symptoms from occurring, ameliorating or preventing the underlying systemic causes of symptoms, inhibiting the disorder or disease, e.g., arresting the development of the disorder or disease, relieving the disorder or disease, causing regression of the disorder or disease, relieving a condition caused by the disease or disorder, or stopping the symptoms of the disease or disorder.

Pharmaceutical or therapeutic compositions disclosed herein may be formulated for administration by any suitable route of delivery, such as oral, parenteral, rectal, nasal, topical, or ocular routes, or by inhalation. By way of example but not by way of limitation, PD-1 blockade therapeutic compositions may be administered intravenously.

A “subject in need of treatment” may include a subject having a disease, disorder, or condition that is responsive to an inhibitor of PD-1 activity (e.g., a PD-1 blockade). For example, a “subject in need of treatment” may include a subject having a cell proliferative disease, disorder, or condition such as cancer. Cancers may include, but are not limited to malignant glioma (e.g., glioblastoma multiforme (GBM)), melanoma, Merkel cell carcinoma, lung cancer (non-small cell lung cancer and small cell lung cancer), renal cancer (e.g., renal cell carci-noma), Hodgkin lymphoma, hepatocellular carcinoma, colorectal carcinoma (microsatellite instability-high), bladder cancer, pancreatic cancer, head and neck cancer, squamous cell carcinoma, diffuse large B-cell lym-phoma, prostate cancer, ovarian cancer, endometrial carci-noma, breast cancer (e.g., triple-negative breast cancer), and any cancer subtype showing microsatellite instability.

A “subject in need of treatment” may include a subject having a malignant glioma, such as GBM. In some embodiments, a “subject in need of treatment” may include a subject having or diagnosed with malignant glioma, for example, a subject may have recurrent GBM, and elevated levels of p-ERK in a sample of the tumor tissue as compared to a control or reference subject or sample. By way of example, but not by way of limitation, a high, higher, or elevated level of p-ERK can include a higher density of p-ERK⁺ cells within tumoral regions of a biopsy sample as compared to a control or a reference sample.

As used herein, the term “reference level” refers to the level of expression of a biomarker in a known sample against which another test sample is compared. A reference level can be obtained, for example, from a known cancer sample from a different individual or group of individuals (e.g., not the individual being tested). The reference level may be determined before and/or after a treatment, e.g., from samples obtained from the same subject before and/or after treatment. A known sample can also be obtained by pooling samples from a plurality of individuals to produce a reference level over an averaged population. A “level” can be an amount of nucleic acid expression, protein expression, level of a biomarker (e.g., p-ERK), or a percentage of cells expressing a biomarker, e.g., in a stained, tumor biopsy sample.

As used herein “control,” as in “control subject” or “control sample” has its ordinary meaning in the art, and refers to a sample, or a subject, that is appropriately matched to the test subject or test sample and is treated or not treated as appropriate.

Tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

Malignant Gliomas

Malignant gliomas include glioblastomas, anaplastic astrocytomas, anaplastic oligodendrogliomas and anaplastic oligoastrocytomas, and some less common tumors such as anaplastic ependymomas and anaplastic gangliogliomas. Malignant gliomas have high morbidity and mortality. Even with optimal treatment, median survival is only 12-15 months for glioblastomas and 2-5 years for anaplastic gliomas. Disclosed herein are methods and compositions useful to treat malignant gliomas. While the inventive methods and compositions are exemplified herein using the most comment and aggressive form of malignant glioma, glioblastoma, as a model, the invention is not intended to be limited to this single form of malignant glioma, and the treatment of other forms of malignant glioma is also contemplated.

Glioblastoma, also known as glioblastoma multiforme (GBM) represents the most frequently occurring and among the most aggressive form of primary malignant brain tumors, accounting for 54% of glioma. Glioblastoma is an aggressive type of cancer that can occur in the brain or spinal cord. Glioblastoma forms from astrocytes that support nerve cells. Glioblastoma can occur at any age but tends to occur more often in older adults. It can cause worsening headaches, nausea, vomiting and seizures.

A patient with neurological symptoms will first be given a physical exam that includes neurologic function tests (reflexes, muscle strength, eye and mouth movement, coordination, and alertness). If a tumor is suspected, the patient will have imaging tests so that a brain abnormality can be detected. These tests may include magnetic resonance imaging (MM) and computerized tomography (CT) scans to produce detailed images of the brain and spine and allow doctors to detect the presence of a tumor. MM scans typically provide the best images of glioblastoma multiforme; those scans are usually done with a contrast agent (dye) to help distinguish the tumor from normal brain tissue. A surgical biopsy may be performed to help confirm the diagnosis. In this procedure, a neurosurgeon extracts a small sample of abnormal cells to test in a pathology laboratory. The main clue to a tumor's being glioblastoma multiforme is the cell necrosis, or cell death, that is characteristic of GBM.

GBM can be very difficult to treat, and a cure is often not possible. Treatments may slow progression of the cancer and reduce signs and symptoms. Treatment options include surgery, radiation, chemotherapy, tumor treating fields therapy, and targeted drug therapy,

A sub-analysis in an international randomized trial by the European Organization for Research and Treatment of Cancer (EORTC) and the National Cancer Institute of Canada (NCIC) compared the results of radiation therapy (RT) alone with those of concomitant RT and temozolomide (TMZ) and found that the addition of TMZ to RT for newly diagnosed GBM resulted in a significant survival benefit. A significant survival benefit of TMZ administration was also found in the subgroup analysis of the 5-year survival data of the EORTC/NCIC trial. Since then, TMZ has been the first-line chemotherapeutic agent for GBM. However, despite aggressive treatment including surgery, radiation therapy (RT), and adjuvant TMZ-based chemotherapy, the prognosis of patients with GBM has been extremely poor, with a median survival of 14.6 months from diagnosis. The median progression-free-survival (PFS) after standard therapy is 6-9 months, demonstrating that current treatment options are palliative rather than curative.

The prognosis for recurrent glioblastoma is much worse than for GBM, with most patients dying within six months. In general, treatment of recurrent GBM may involve repeated resection, focal RT, chemotherapy, or experimental therapies. There has been very modest progress in the treatment of recurrent GBM and there are no standard treatment recommendations for recurrent GBM. In view of the unfavorable survival outlook with currently available treatment modalities, new methods for the treatment for GBM and recurrent GBM are desirable.

Extracellular Signal-Regulated Kinases (ERKs)

Extracellular signal-regulated kinases (ERKs), also known as MAP kinases, refer to the widely expressed protein kinase intracellular signaling molecules that are involved in numerous cellular functions including the regulation of meiosis, mitosis, and post-mitotic functions in differentiated cells. Many different stimuli, including growth factors, cytokines, infection, ligands for heteromeric G protein-coupled receptors, transforming agents, and carcinogens, activate the ERK pathway. Also known as MAPK3 and MAPK1, the MAP kinases FRIG and ERK2 are 44- and 42-kDa serine/threonine kinases, respectively, with 90% sequence identity in mammals. Initially isolated and cloned as kinases activated in response to insulin and -Nerve Growth Factor (NGF), ERK1 and ERK2 are both expressed in most, if not all, mammalian tissues, with ERK2 levels generally higher than ERK1. Knock-out studies in mice demonstrate that either ERK may at least partially compensate for the other's loss. Because of their high degree of similarity, ERK-1 and ERK-2 are usually considered to be functionally redundant, and as used herein, the term “ERK” includes both ERK-1 and ERK-2. ERK-1 and -2 are activated by phosphorylation to yield p-ERK (phosphorylated ERIK). Because of the rather broad nature of its substrate recognition, the ERKs can phosphorylate a large number of proteins after their activation. Targets or ERK. activation are often regulatory in nature and are located both in the cytoplasm and the nucleus.

The amino acid sequences of ERK1 and ERK2 are provided as SEQ ID NO:1 and SEQ ID NO:2, respectively.

```
SEQ ID NO: 1
                10 20 30 40
MAAAAAQGGG GGEPRRTEGV GPGVPGEVEM VKGQPFDVGP
                50 60 70 80
RYTQLQYIGE GAYGMVSSAY DHVRKTRVAI KKISPFEHQT
                90 100 110 120
YCQRTLREIQ ILLRFRHENV IGIRDILRAS TLEAMRDVYI
              130 140 150 160
VQDLMETDLY KLLKSQQLSN DHICYFLYQI LRGLKYIHSA
              170 180 190 200
NVLHRDLKPS NLLINTTCDL KICDFGLARI ADPEHDHTGF
              210 220 230 240
LTEYVATRWY RAPEIMLNSK GYTKSIDIWS VGCILAEMLS
              250 260 270 280
NRPIFPGKHY LDQLNHILGI LGSPSQEDLN CIINMKARNY
              290 300 310 320
LQSLPSKTKV AWAKLFPKSD SKALDLLDRM LTFNPNKRIT
              330 340 350 360
VEEALAHPYL EQYYDPTDEP VAEEPFTFAM ELDDLPKERL
              370
KELIFQETAR FQPGVLEAP
SEQ ID NO: 2
                10 20 30 40
MAAAAAAGAG PEMVRGQVFD VGPRYTNLSY IGEGAYGMVC
                50 60 70 80
SAYDNVNKVR VAIKKISPFE HQTYCQRTLR EIKILLRFRH
                90 100 110 120
ENIIGINDII RAPTIEQMKD VYIVQDLMET DLYKLLKTQH
              130 140 150 160
LSNDHICYFL YQILRGLKYI HSANVLHRDL KPSNLLLNTT
              170 180 190 200
CDLKICDFGL ARVADPDHDH TGFLTEYVAT RWYRAPEIML
              210 220 230 240
NSKGYTKSID IWSVGCILAE MLSNRPIFPG KHYLDQLNHI
              250 260 270 280
LGILGSPSQE DLNCIINLKA RNYLLSLPHK NKVPWNRLFP
              290 300 310 320
NADSKALDLL DKMLTFNPHK RIEVEQALAH PYLEQYYDPS
              330 340 350 360
DEPIAEAPFK FDMELDDLPK EKLKELIFEE TARFQPGYRS
```

Methods to detect phosphorylated ERK are available and are well known in the art, for example, PerkinElmer's AlphaScreen® SureFire™ ERK Assay (high throughput capability using bead proximity-based AlphaScreen technology); ELISA (Enzyme linked immunosorbent assays); Meso-Scale Discovery Assays (Electrochemiluminesence-based method providing medium to high throughput screening); LICOR (Infrared fluorescence-based method providing medium to high throughput screening); Western Blot Analysis; and the use of antibodies (e.g., monoclonal antibodies directed to p-ERK) followed by visualization with, for example, a secondary and/or tertiary labeled antibody.

Methods to Identify Likely Responders and Likely Non-Responders to PD-1 Blockade Therapy

Disclosed herein are methods for identifying subjects, diagnosed as having a malignant glioma, such as glioblastoma (GBM), as likely responders, or as likely non-responders, to treatment with a programmed death protein 1 (PD-1) blockade. In some embodiments, the subjects are identified by determining the level of phosphorylated extracellular-signal-regulated kinase (p-ERK) in a tumor sample from the subject (e.g., a GBM tumor sample). In some embodiments, the subject has GBM; in some embodiments, the GBM is recurrent.

As used herein, the term “PD-1 blockade” refers to a therapy that inhibits the activity of programmed cell death protein 1 (PD-1) and/or its ligand, PD-L1. Such compounds are referred to herein as PD-1 inhibitors, or PD-L1 inhibitors, depending on the mode of action (by way of example, an antibody that binds PD-1 would be considered a PD-1 inhibitor; an antibody that binds PD-L1 would be considered a PD-L1 inhibitor; a small molecule that blocks PD-L1 would be considered a PD-L1 inhibitor, etc.). Exemplary therapeutics employed to block PD-1 or its ligand include but are not limited to monoclonal antibodies Nivolumab (anti-PD1; Bristol-Myers Squibb), Pembrolizumab (anti-PD1; Merck), Atezolizumab (anti-PD-L1; Genentech/Rothe), Avelumab (anti-PD-L1; EMD Serono/Merck&Pfizer) and Durvalumab (anti-PD-L1; AstraZeneca). Other monoclonal antibodies include Cemiplimab (anti-PD1; Regeneron Pharmaceuticals), Spartalizumab (PDR001) is a PD-1 inhibitor developed by Novartis to treat both solid tumors and lymphomas, which as of 2018 has entered. Phase III trials; Camrelizumab (SHR1210) is an anti-PD-1 monoclonal antibody introduced by Jiangsu HengRui Medicine Co., Ltd. that recently received conditional approval in China for the treatment of relapsed or refractory classical Hodgkin lymphoma; Simi (IBI308), a human anti-PD-1 antibody developed by innovent and Eli Lilly for patients with non-small cell lung cancer; Tislelizumab (BGB-A317) is a humanized IgG4 anti-PD-1 monoclonal antibody in pivotal Phase 3 and Phase 2 clinical trials in solid tumors and hematologic cancers; Toripalimab (JS 001) is a humanized IgG4 monoclonal. antibody against PD-1 under clinical. investigation; Dostarlimab (TSR-042, WBP-285) is a humanized monoclonal anti-body against PI)-1 under investigation by GlaxoSimthKline; INCMGA00012 (MGA012) is a humanized IgG4 monoclonal antibody developed by Incyte and MacroGenics; AMP-224 by AstraZeneca/MedImmune and GlaxoSmith Kline; AMP-514 (MFDI0680) by AstraZeneca. Exemplary PD-L1 antibodies and PD-L1 inhibitors include KN035 a PD-L1 antibody with subcutaneous formulation currently under clinical evaluations in the US, China, and. Japan; CK-301 by Checkpoint Therapeutics; AUNP12 is a 29-mer peptide as the first peptic PD-1/PD-L1 inhibitor developed by Aurigene and Laboratoires Pierre Fabre that is being evaluated in a clinical trial, following promising in vitro results; CA-170, discovered by Aurigene/Curis as the PD-L1 and VISTA antagonist, was indicted as a potent small molecule inhibitor in vitro; BMS-986189 is a macrocyclic peptide being evaluated by Bristol-Myers Squibb; and, Balstilimab (AGEN2034) a human monoclonal immunoglobulin G4 that blocks PD-1 developed by Agenus.

By way of example but not by way of limitation, in some embodiments, a biopsy sample from a subject diagnosed with, or suspected of having a glioma such as GBM is treated to visualize cells expressing p-ERK. The number of cells expressing p-ERK can then be compared to the total number of cells in the sample or in the visualization field, e.g., to define the percentage of p-ERK⁺ cells in the sample or in the visualization field. In some embodiments, such a percentage describes a p-ERK level in the sample. In some embodiments, a p-ERK level is described by the intensity of the IHC staining and number of cells expressing p-ERK in relative terms, e.g., as numerical values: 0=negative; 1=weak positive; 2=moderately positive; 3=strong positive. A complementary method for p-ERK IHC assessment is the quantification of p-ERK⁺ cell density in tumoral regions. Once a certified pathologist outlines the tumor regions in whole-slide images for each case, several representative regions of interest (ROI) have to be selected to quantify p-ERK⁺ cell density using a quantitative image analysis software. Parameters such as nuclear size, hematoxylin and DAB chromogen intensity staining need to be adjusted to identify cells with p-ERK⁺ staining in segmented nuclei and their surrounding cytoplasmic regions. The same software parameters will need to be used to analyze all cases.

In some embodiments, a p-ERK level is determined by semi-quantitative image analysis of p-ERK⁺ cell density; in some embodiments, a p-ERK level is determined by a pathologist, for example, in a clinical setting.

In some embodiments, a reference level, or threshold level of p-ERK expressing cells is determined. By way of example, p-ERK tumor samples from a cohort of GBM subjects can evaluated by semi-quantitative image analysis of p-ERK⁺ cell density. The median of all the values of the p-ERK⁺ cell density as determined by quantitative image analysis of all patient specimens analyzed may be used at the cutoff or threshold between high or low p-ERK groups.

In some embodiments, the determination of subject's p-ERK level allows for categorization of the subject as a likely responder or as a likely non-responder to a particular therapy such as PD-1 blockade. For example, a subject with recurrent GBM having a high p-ERK level (e.g., having a tumor sample that exhibits a moderate or strong positive for p-ERK⁺ cells) is categorized as a “likely responder.” It is anticipated that a likely responder will respond favorably and positively to a PD-1 blockade therapy and will for example, exhibit an improvement or abatement of symptoms, and/or an increase in longevity. A subject with recurrent GBM having a low p-ERK level (e.g., having a tumor sample that exhibits a negative or weak positive for p-ERK⁺ cells) is categorized as a “likely non-responder.” It is anticipated that a likely non-responder will show no improvement or abatement of symptoms, and no increase in longevity in response to PD-1 blockade therapy.

### Methods of Treatment

In some embodiments, a subject is diagnosed with GBM, and has a level of p-ERK indicating that the subject is a likely responder to a PD-1 blockade therapy. Thus, in some embodiments, the subject is administered one or more therapeutic PD-1 blockade therapies, alone or in combination with one or more additional therapeutic agents or therapeutic methodologies, such as surgery, chemotherapy, targeted therapy, radiation, vaccine therapies, oncolytic virotherapy, viral-mediated immunothherapy, gene immunotherapy, adoptive cell transfer therapy including but not limited to CAR T-cell therapy, or with other immune checkpoint inhibitors, such as IDO1, OX40, LAG3, KIR, 4-IBB, TIGIT, TIM-3, CTLA-4 monoclonal antibodies, small molecule pharmacology agents, and chemotherapy that could serve as an immune modulator.

Dosage and administration of PD-1 blockade therapy, alone or in combination with additional therapeutic agents can be determined by a skilled artisan using methods that are standard in the art, based on patient age, weight, sex, race, overall health, stage of the disease (e.g., GBM), etc. By way of example, but not by way of limitation, in some embodiments, a dosage range of from about 0.1 to about 50 mg/kg, from about 1 to about 10 mg/kg, from about 1 to about 20 mg/kg, from about 1 to about 30 mg/kg, or from about 1 to about 40 mg/kg. In some embodiments, the PD-1 blockade therapeutic is provided as a single administration. In some embodiments, the PD-1 blockade therapeutic is provided as multiple administrations. In some embodiments, the PD-1 blockade therapeutic is administered daily, every other day, twice per week, weekly, bi-weekly or monthly. In some embodiments, the PD-1 blockade therapeutic is administered intravenously.

The PD-1 blockade therapeutic may be administered alone, or simultaneously with, or sequentially with, one or more additional therapeutic agents or treatment methodologies.

p-ERK is Informative for Response to PD1 Blockade in Recurrent GBM

Immune checkpoint blockade has seen an unparalleled expansion in cancer therapy leading to long-term remissions and has been adopted as the standard of care of advanced melanoma, non-small cell lung cancer, clear-cell renal cell carcinoma, solid tumors with DNA mismatch repair deficiency or microsatellite instability, and an increasing number of other cancers. In contrast, its success has been limited in glioblastoma (GBM). Whereas several negative trials showed a lack of efficacy of this type of immunotherapy for GBM in general, clinical responses in subsets of patients have also been reported. This highlights the importance of personalized medicine for successful implementation of this immunotherapy for GBM. Unfortunately to date there are no reliable predictive biomarkers for response to PD-1 blockade in GBM, and predictive biomarkers in other cancers, have not proven as informative for this disease.

In our previous work (Zhao et al., Nature Med 2019), we performed an analysis of recurrent GBM patients treated with adjuvant PD-1 blockade. In this study, we reported that activating mutations of BRAF/PTPN11 are associated with response and overall survival in the context of this immunotherapy. Whereas this finding was important, using these mutations to guide patient care is not practical in a clinical setting, as these only occur in 2-3% of GBM, and in 30% of responder tumors. Thus, the majority of patients that respond cannot be identified, and therefore, this therapy cannot be incorporated into the management of these cases. The possibility of efficacious implementation of PD-1 blockade for a subset of recurrent GBM is of upmost importance as all GBM patients eventually recur, and at this point, there are no treatment options.

Given that BRAF and PTPN11 are upstream regulators of MAPK pathway, we investigated how activation of this signaling cascade relates to response to PD-1 blockade immunotherapy in recurrent GBM. Here, we report that MAPK signaling determined by ERK1/2 phosphorylation (p-ERK), is informative for response to PD-1 blockade in recurrent GBM. p-ERK prediction of response to PD-1 blockade applies to the majority of patients that enjoy long term survival, including tumors that do not harbor mutations of BRAF/PTPN11.

As demonstrated in Example 1, below, we report a novel and robust predictive biomarker for immunotherapy response in cancer, and in particular in a tumor for which there has not been overall success with this therapeutic modality. This biomarker is easy to implement and does not carry with it the cost or delays associated with the use of next-generation sequencing analysis for clinical practice. Our results indicate that the majority of recurrent GBM patients that enjoy response to PD-1 blockade can be identified. This is important as this therapy is otherwise difficult to adopt. On the other hand, this is relevant as at recurrence, GBM patients do not have effective therapeutic options.

### Advantages and Applications

To our knowledge, p-ERK constitutes the most robust indicator of response to immunotherapy for gliomas, and a completely novel predictive biomarker that has not been described in cancer. Example 1, below, illustrates the following.

There are multiple ongoing trials in which immune checkpoint blockade is being combined with other immunotherapy modalities such as oncolytic viruses, gene therapy for immunomodulation, and others. Our results might allow these other trials to identify a signal of response within the otherwise routinely reported lack of overall efficacy for novel therapies in recurrent GBM.

p-ERK and MAPK activation might be predictive of response to other forms of immunotherapy such as CAR T-cells, and could be informative for response to immunotherapy in other cancers. Our study will prompt this investigation.

Our study indicates that there is an association between oncogenic signal in tumor cells (i.e. MAPK), and the microenvironment. In particular, we found an associated phenotype of infiltrating myeloid cells. This observation adds to the growing literature supporting the idea that response to PD-1 blockade and immunotherapy in gliomas might be influenced by myeloid cells, and not directly depend on pre-existing T-cell infiltration.

Phosphorylation of ERK1/2 assessed by IHC can be used a predictive marker of response to immune checkpoint inhibitors and other types of immunotherapy in gliomas.

Immune checkpoint inhibitors are potential alternatives to the standard of care temozolomide in primary glioblastomas.

At recurrence, the therapeutic options available are limited for glioblastoma. Therefore, immune checkpoint inhibitors represent a potential effective treatment for patients having high p-ERK tumors.

Herein, data is presented to indicate that p-ERK is predictive of response to PD-1 blockade in glioblastoma.

### EXAMPLES

The following Examples are illustrative and should not be interpreted to limit the scope of the claimed subject matter.

### Example 1—ERK 1/2 Phosphorylation Predicts Survival Following Anti-PD-1 Immunotherapy in Recurrent Glioblastoma

### Abstract

PD-1 checkpoint inhibition has led to remarkable clinical responses in several cancer types. Whereas PD-1 blockade has not shown an overall survival (OS) benefit for glioblastoma (GBM) patients, a subset of them exhibit long-term responses to this immunotherapy. Previously, we reported an enrichment of BRAF/PTPN11 activating mutations in 30% of recurrent GBMs that responded to PD-1 blockade, but the molecular profile of the majority of responders remained elusive. Given that BRAF and PTPN11 promote MAPK/ERK signaling, we investigated whether activation of this pathway is associated with response to PD-1 inhibitors in recurrent GBM, including patients that do not harbor BRAF/PTPN11 mutations. Immunohistochemistry for ERK1/2 phosphorylation (p-ERK), a marker of MAPK/ERK pathway activation, was performed in a discovery cohort including pre-treatment specimens of 29 recurrent GBM patients treated with adjuvant PD-1 blockade, and 33 patients who did not undergo immunotherapy. p-ERK was predictive of response and OS following PD-1 blockade. Yet p-ERK was not associated with OS in patients not treated with immunotherapy. p-ERK was also associated with OS in a validation GBM cohort treated with adjuvant anti-PD-1 therapy. Single-cell RNA-seq and multiplex-immunofluorescence analyses revealed that p-ERK was mainly localized in tumor cells and high p-ERK GBMs contained tumor-infiltrating myeloid cells and microglia with elevated expression of MHC class II and associated genes. Thus, our findings indicate that ERK1/2 activation in recurrent GBM is predictive of response to PD-1 blockade and is associated with a distinct myeloid cell phenotype.

### Introduction

The prognosis of glioblastoma (GBM) patients is poor with a median overall survival (OS) of around 21 months¹. Whereas radiation, chemotherapy and tumor-treating fields are established treatments at initial diagnosis; at recurrence, there are no effective therapies. This is attributed partially to the notorious molecular and microenvironmental heterogeneity of GBM^{2,3}, which contributes to erratic and unpredictable responses to specific therapies. Thereby, the variable response to therapy across GBM patients often manifests as negative clinical trials in which an elusive subset of patients exhibit response.

Immune checkpoint blockade has seen an unparalleled expansion in cancer therapy leading to long-term remissions even in cases of advanced metastatic disease. It has been adopted as the standard of care for advanced melanoma, non-small cell lung cancer, clear-cell renal cell carcinoma, solid tumors with DNA mismatch repair deficiency or microsatellite instability (MSI), and an increasing number of cancers⁴. In contrast, its success has been limited in GBM⁵⁻⁷, partly due to the diverse and iterative mechanisms of intrinsic and iatrogenic immunosuppression. These include tumor infiltration by immunosuppressive cells, defects in the antigen processing and presentation machinery, sequestration of T cells in the bone marrow, and frequent use of immunosuppressive medications such as corticosteroids among other factors⁸⁻¹³. Whereas negative trials showed an overall lack of efficacy of immune checkpoint blockade for GBM patients, durable clinical responses have been reported in some patients^{5,14-16}. We recently reported an analysis of recurrent GBM patients treated with adjuvant PD-1 blockade where we uncovered molecular features associated with response to this type of immunotherapy¹⁴. Response was based on imaging and pathological criteria; responsive patients exhibited significant prolongation of survival independent of other therapies and known clinical or molecular prognostic variables. BRAF and PTPN11 activating mutations, which are known to drive MAPK/ERK pathway signaling¹⁷⁻¹⁹, were enriched in recurrent GBM patients that responded to PD-1 blockade. Somatic mutations in these MAPK pathway genes were encountered in only approximately 30% of patients who responded to PD-1 inhibitors in our GBM cohort and only 2-3% of GBM harbored such mutations in the unselected population from TCGA. Whereas these mutations might provide biological clues into the GBM biology associated with response to PD-1 inhibitors, these alterations have limited value as a predictive biomarker given that 70% of responder patients were not identified by these mutations¹⁴. In an effort to identify GBM tumors that are susceptible to PD-1 blockade beyond those harboring BRAF or PTPN11 mutations, we explored whether activation of the MAPK pathway is present in recurrent GBM that respond and exhibit prolonged survival following PD-1 blockade. To do this, we analyzed the phosphorylation/activation of ERK1/2, downstream effectors of the MAPK signaling, in specimens from recurrent GBM patients that were treated with PD-1 blockade as well as those who did not undergo immunotherapy. Furthermore, we evaluated the phenotypic and cellular differences of the tumor microenvironment of patients with elevated ERK1/2 activation through multiplex immunofluorescence staining and single-cell RNA-seq (scRNA-seq) of human GBMs.

Results

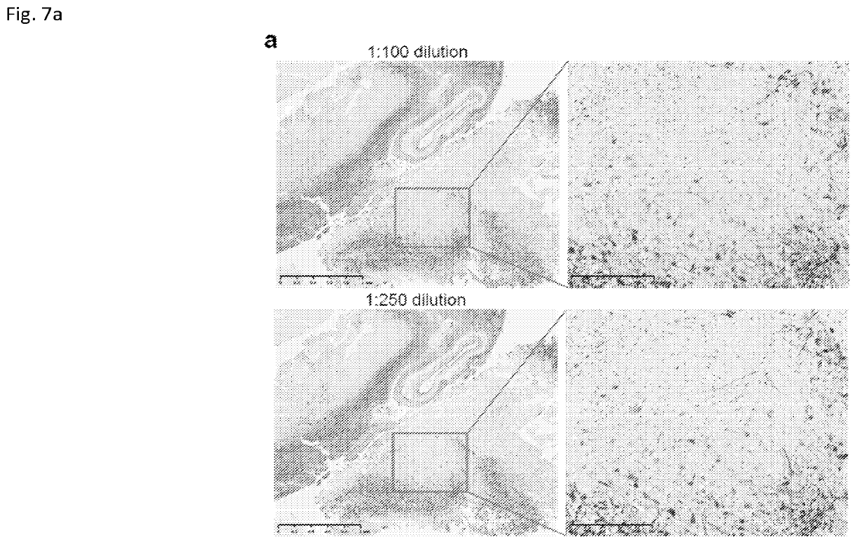

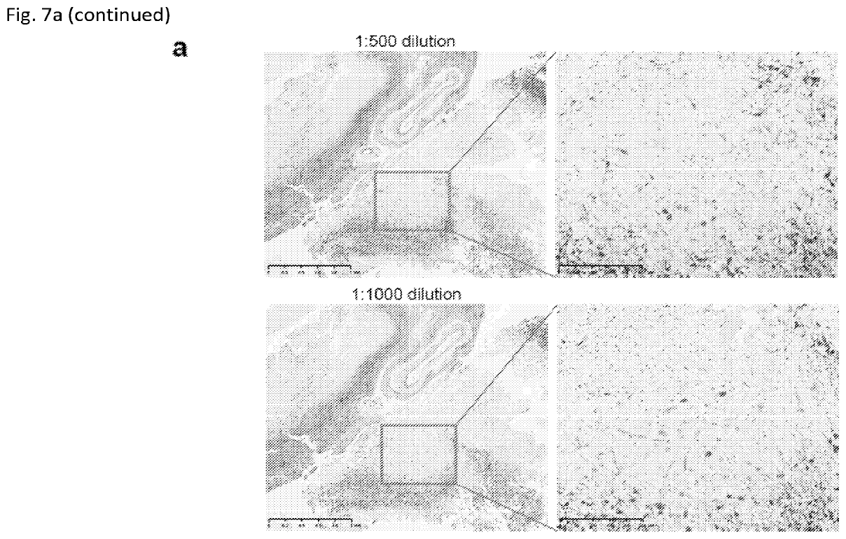

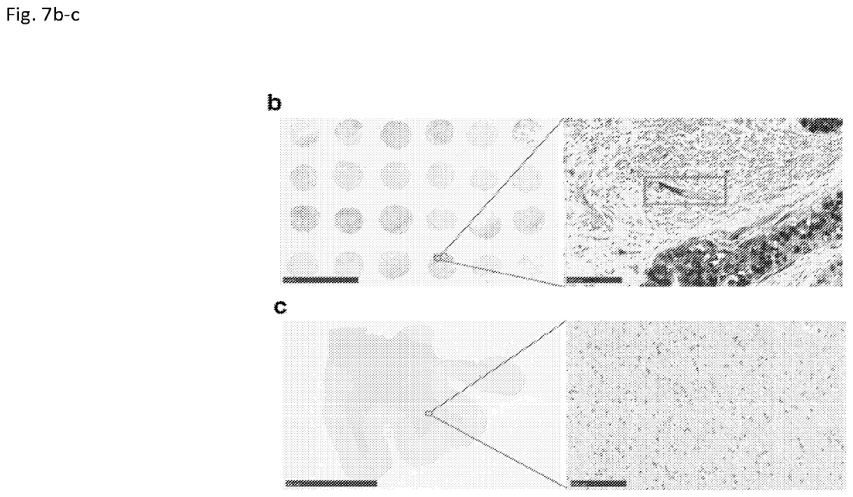

ERK1/2 phosphorylation is predictive of response and overall survival following PD-1 blockade in recurrent glioblastoma. To investigate whether MAPK pathway activation is associated with response to anti-PD-1 therapy beyond the patients identified by BRAF and PTPN11 activating mutations, we evaluated MAPK signaling in GBM through phosphorylation of ERK1/2 (p-ERK), the downstream effectors for this pathway, using immunohistochemistry (IHC)^{20,21}. To maximize the rigor of this analysis, we titrated the p-ERK antibody using tumor samples, non-tumoral human brain and breast cancer microarrays, which are known to express this phospho-antigen²² (FIG. 7a, b, and c).

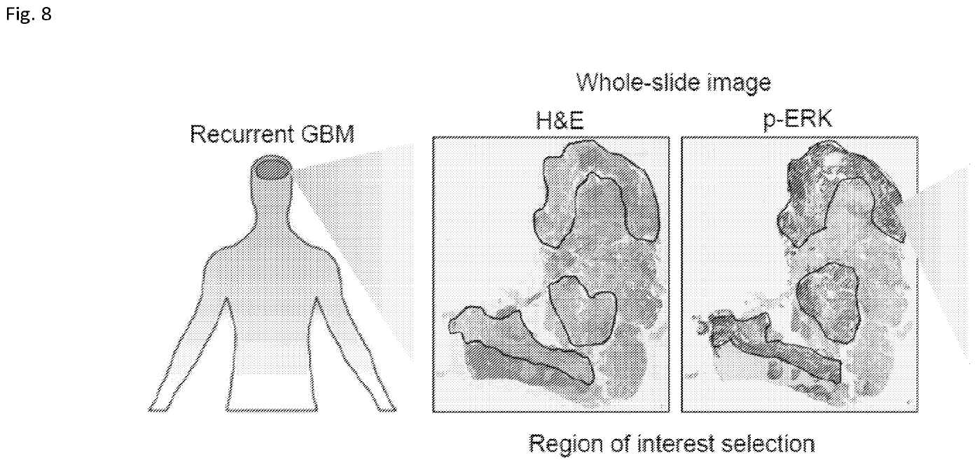

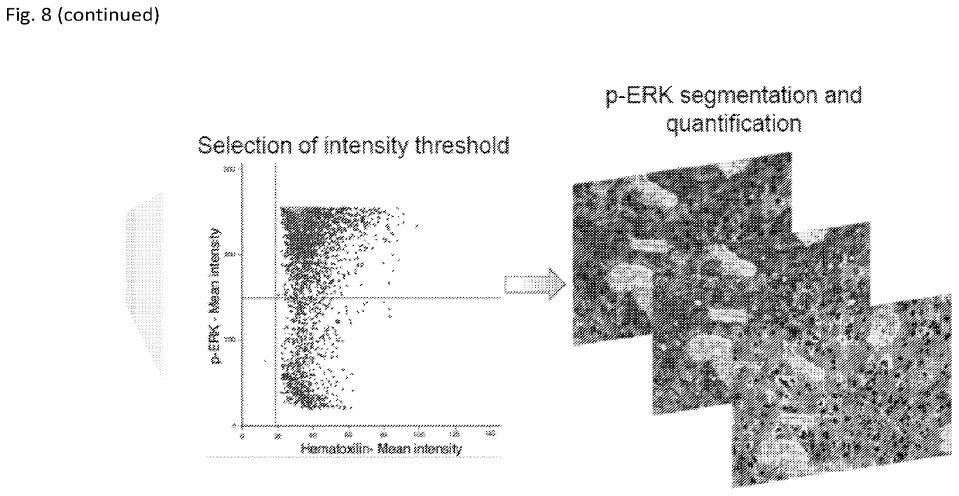

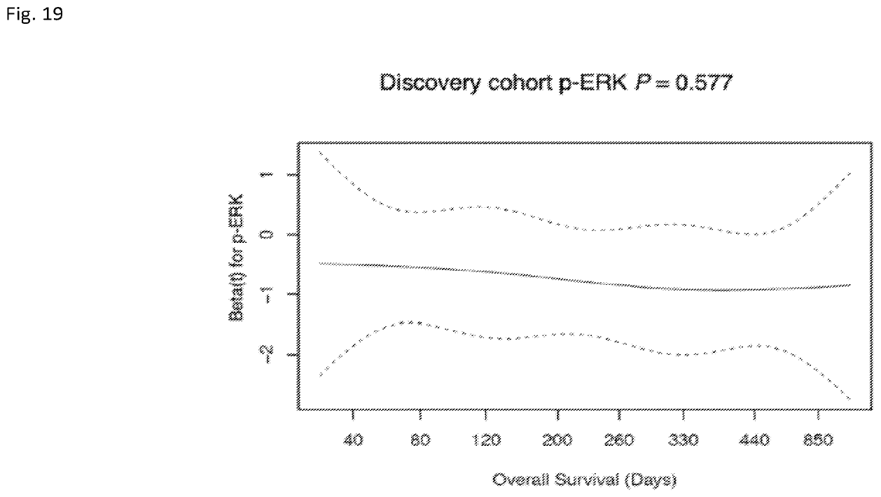

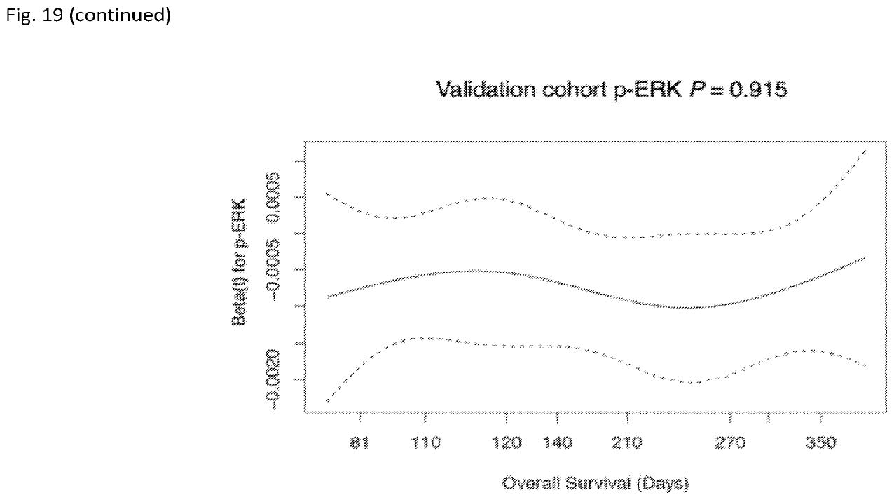

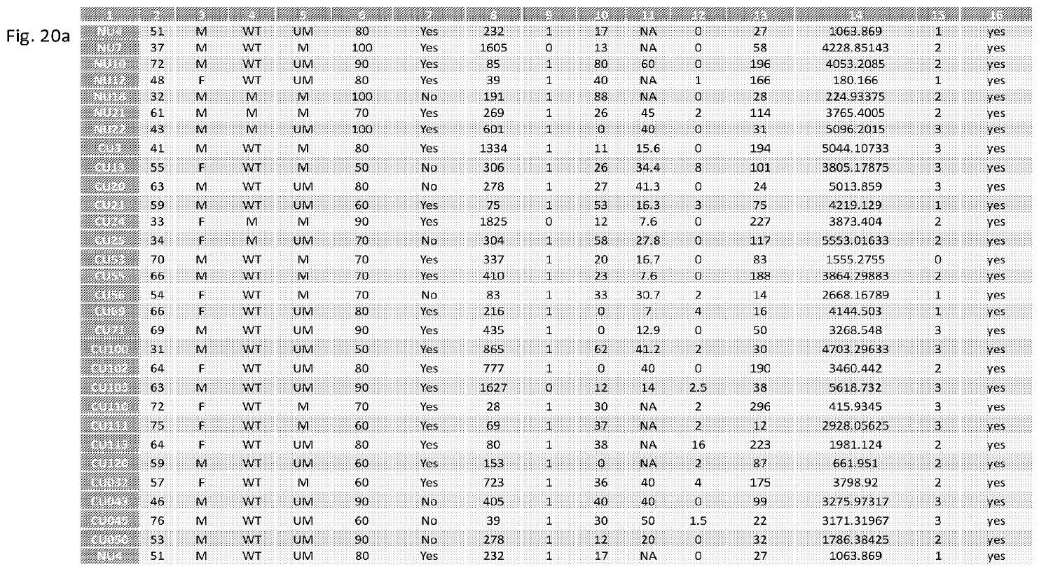

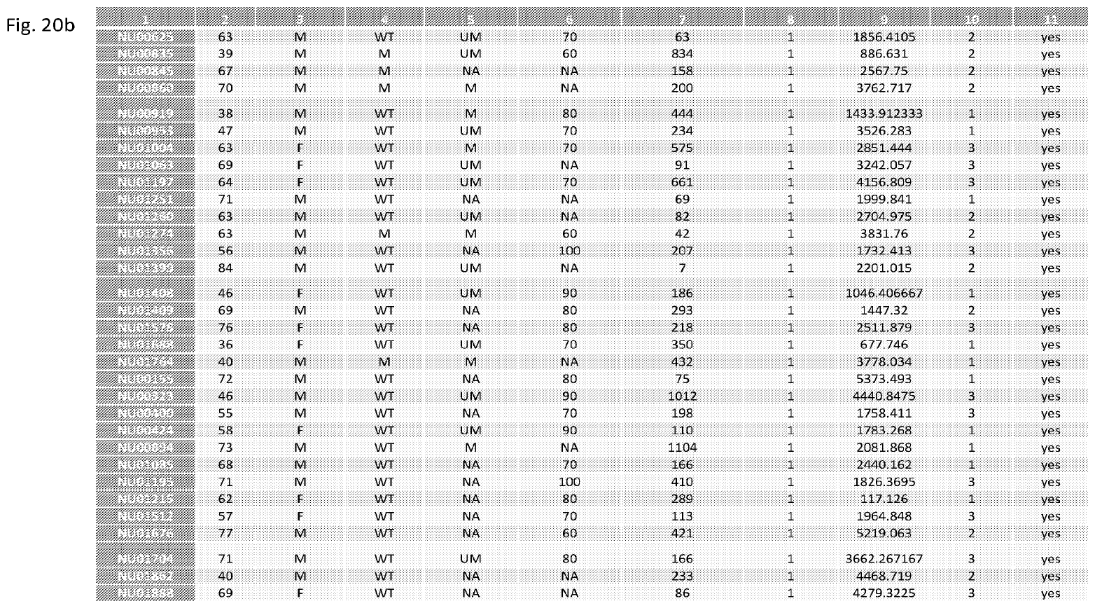

To determine MAPK activity in each specimen, we performed quantitative image analysis of the density of p-ERK⁺ cells within tumoral regions selected by a neuropathologist who was blinded to the clinical data (FIG. 8). p-ERK staining and quantification was performed in tumor specimens from recurrent GBM patients that underwent adjuvant PD-1 blockade (n=29) after tissue was obtained (samples were naïve to immunotherapy), and recurrent GBM patients that underwent surgery at recurrence, but did not undergo immunotherapy (n=33). Patient baseline clinical characteristics and known prognostic factors were similar among both groups (Table 1 at FIG. 19, and FIG. 20). We first investigated whether p-ERK tumor staining was associated with response to PD-1 blockade. For this, patients were classified as responders based on our previous definition of response¹⁴, in which they had to fulfill at least one of the two criteria:
1) Tissue sampled during surgery after PD-1 immunotherapy showed a robust reactive, inflammatory infiltrate and scant to no tumor cells.
2) Tumor size determined by magnetic resonance imaging (MRI) that was either stable or decreasing over at least six months from the initiation of PD-1 blockade.

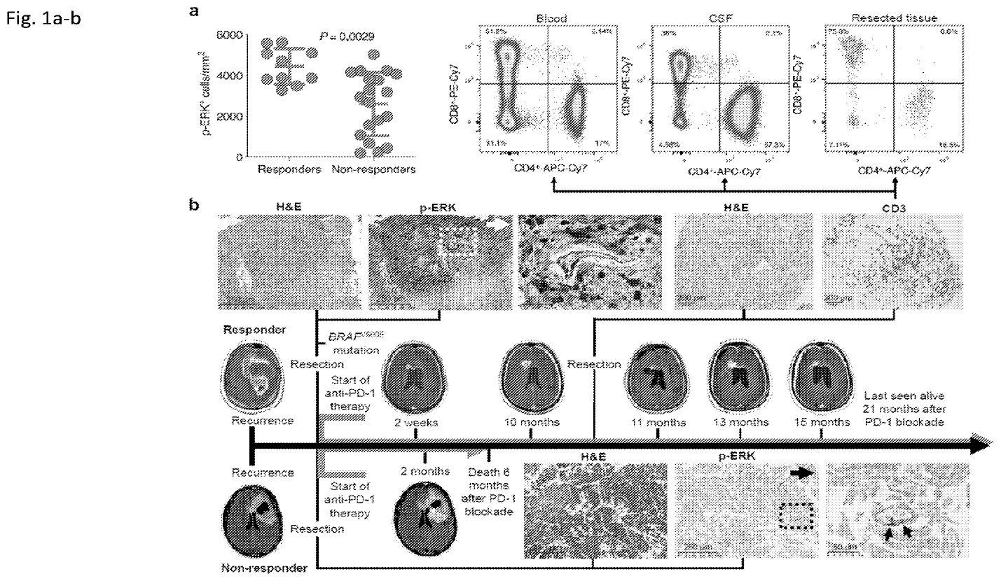

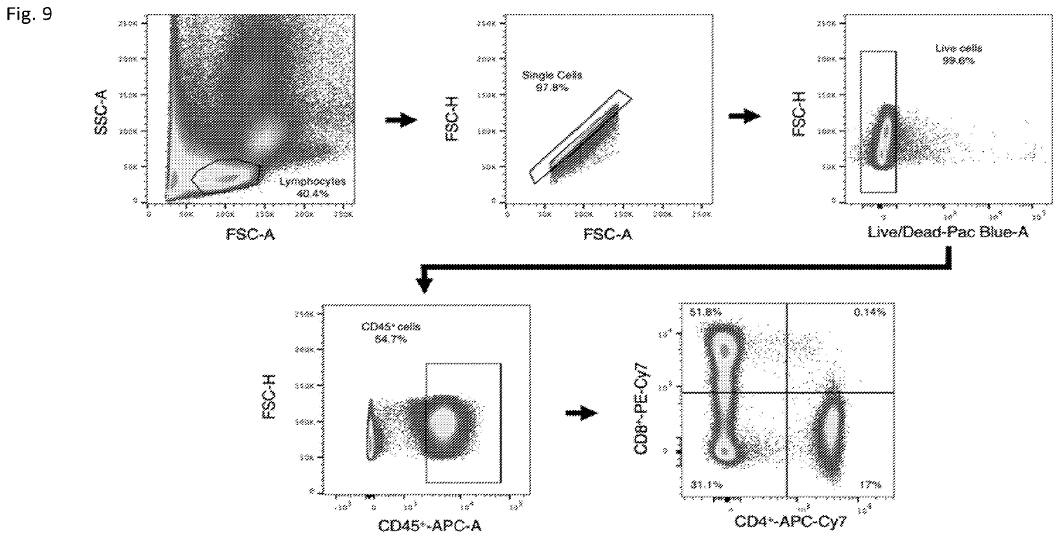

Quantification of p-ERK cell density in tumoral regions revealed that responder patients had significantly increased density of p-ERK⁺ cells relative to non-responder patients (P=0.0029, Mann Whitney U test; FIG. 1a). As a representative example, we present a patient with an MGMT-unmethylated, IDH-wild-type recurrent GBM that had a BRAF^{V600E} mutation who underwent resection followed by PD-1 blockade (FIG. 1b). p-ERK staining showed strong positivity for this tumor. This patient maintained stable disease for 9 months as determined by the RANO criteria²³, yet he developed hydrocephalus and an associated inflammatory profile in his cerebrospinal fluid (CSF). Ten months after surgery and initiation of PD-1 blockade, the growth of a right peri-ventricular enhancing lesion was noticed, which upon biopsy, revealed scant tumor cells and an abundant CD3⁺ T cell infiltrate consisting of both CD4⁺ and CD8⁺ T cells, and a similar lymphocytic pattern in the CSF (FIG. 1b and FIG. 9). The patient continued with the immunotherapy for a total of 21 months from recurrence prior to being lost to follow up. In contrast, we also present a patient with an MGMT-methylated IDH-mutant recurrent GBM that had minimal p-ERK tumor staining, while exhibiting p-ERK staining in endothelium. This patient underwent PD-1 blockade after surgery, but exhibited radiographic progression two months later, and died four months after (FIG. 1b).

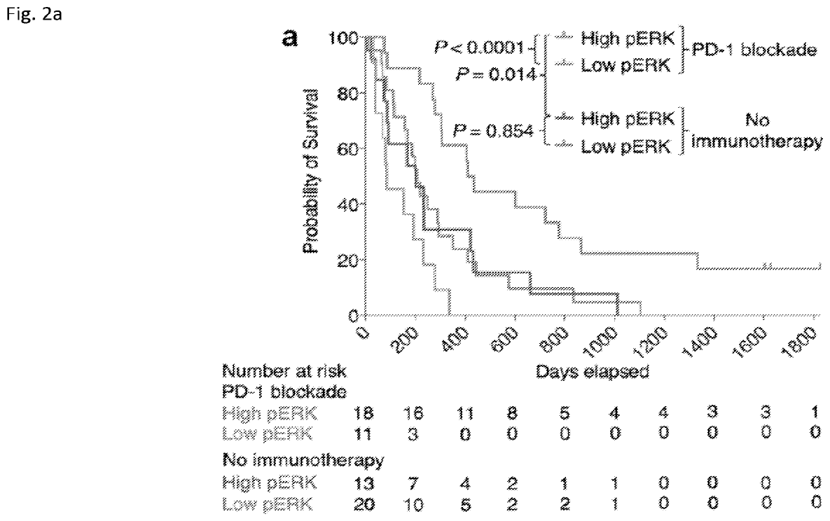

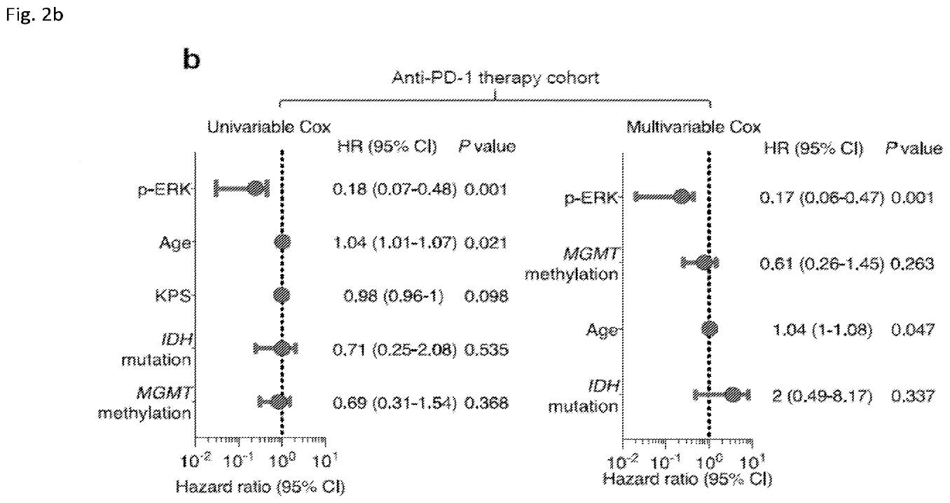

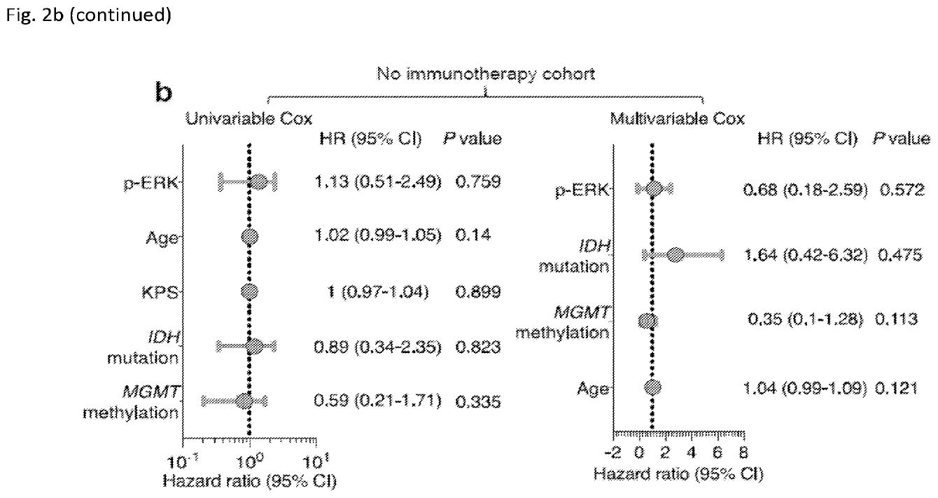

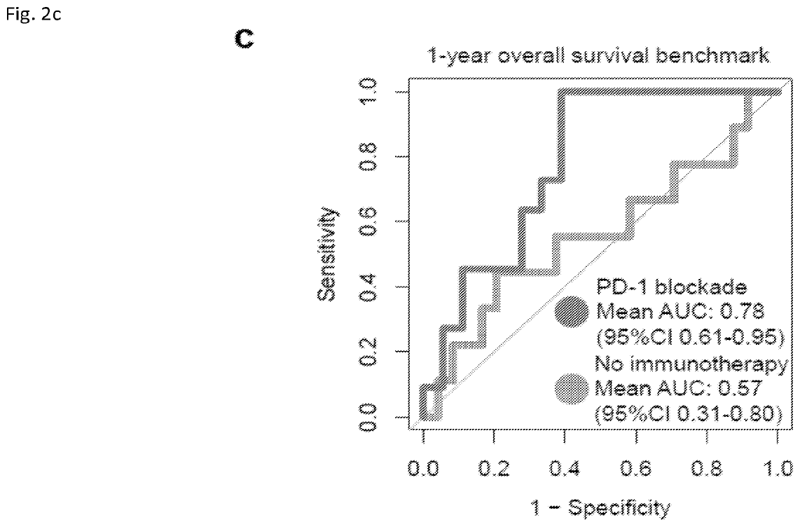

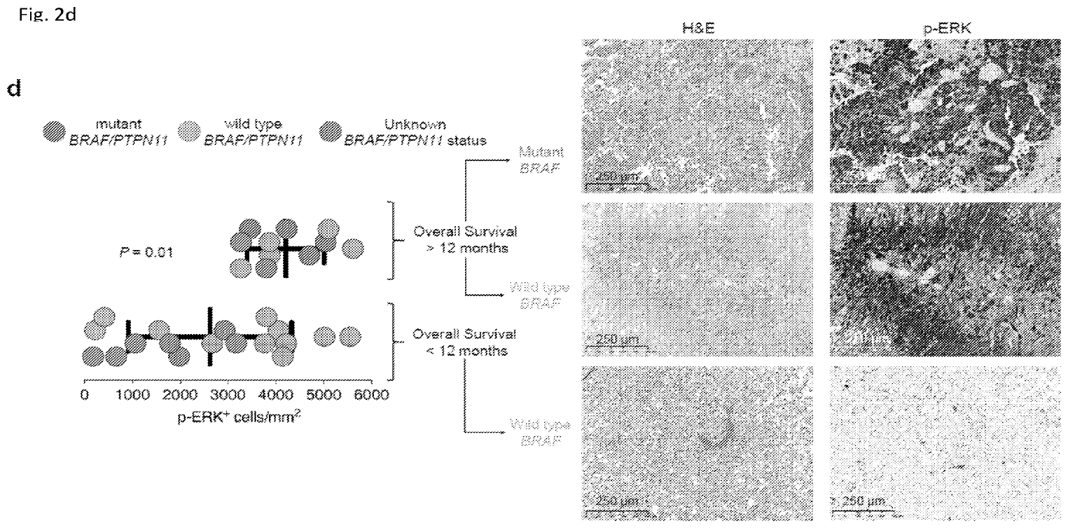

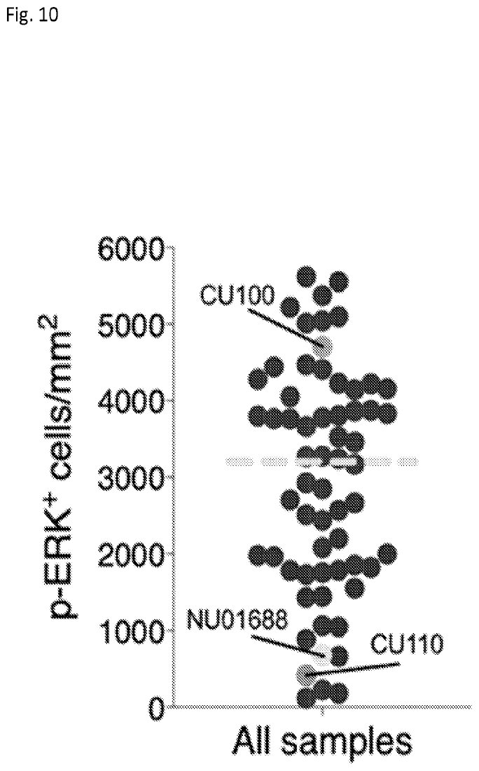

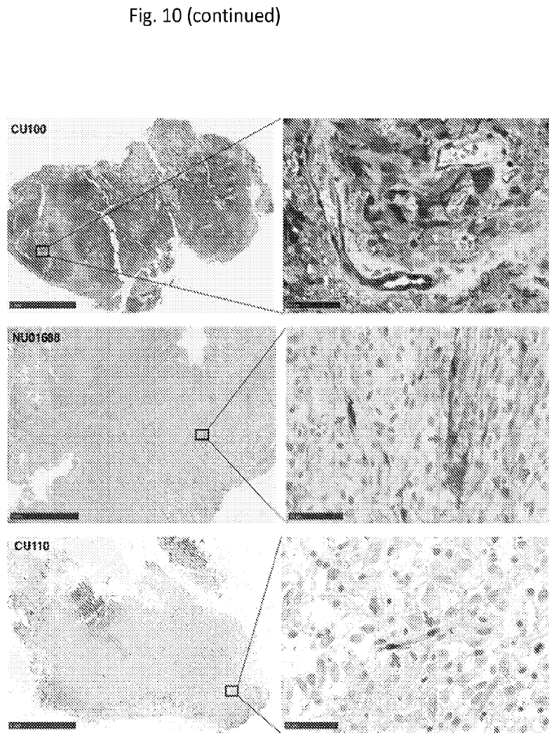

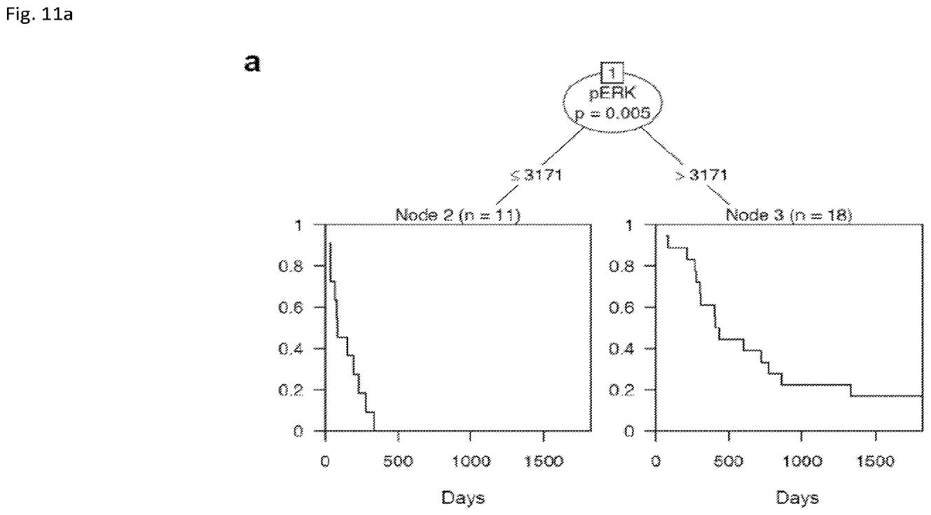

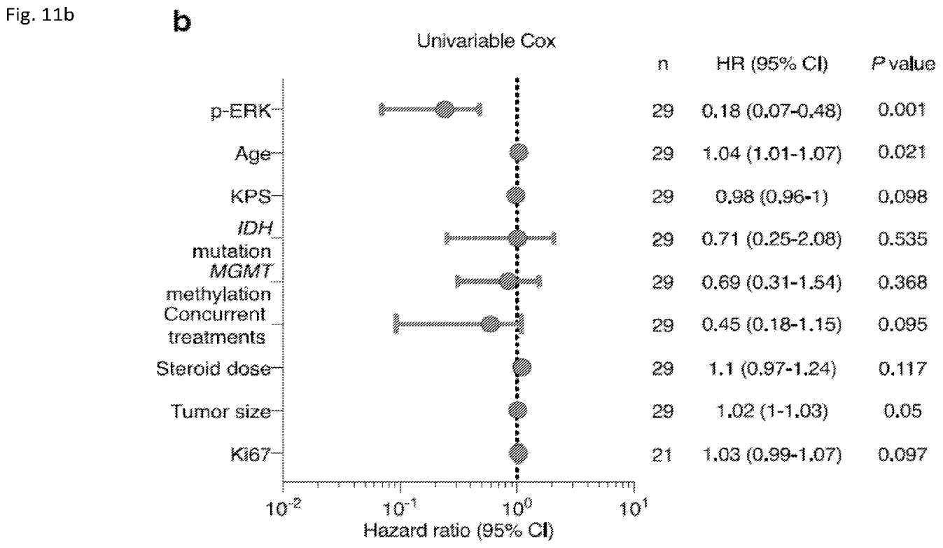

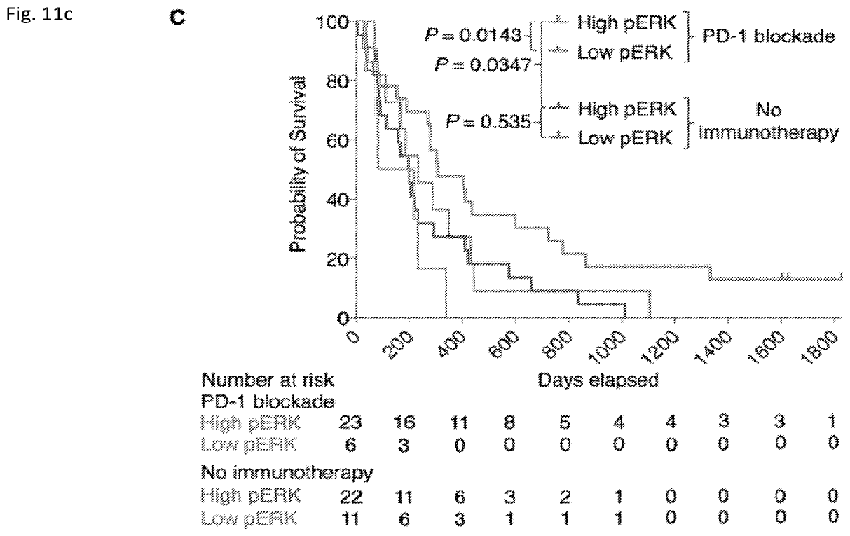

A recognized limitation of Mill for assessment of GBM progression is that brain inflammatory changes derived from immunotherapy may resemble disease progression, a phenomenon designated as pseudoprogression²⁴⁻²⁶, as exemplified on a biopsy specimen obtained 10 months after PD-1 blockade initiation (FIG. 1b). To investigate the predictive value of p-ERK for GBM susceptibility to PD-1 blockade independent of radiographic response, we performed analyses of OS based on the quantification of p-ERK cell density. Recurrent tumor samples from 29 GBM patients treated with PD-1 inhibitors and 33 patients that did not receive immunotherapy were classified to have either high or low p-ERK tumors. The cut-point value used to classify high and low p-ERK tumor groups was derived from the median (3207 p-ERK⁺ cells/mm²) of all the values of the p-ERK cell density determined by quantitative image analysis of all specimens (n=62) (FIG. 10). Based on the date of initiation of PD-1 blockade at recurrence, GBM patients treated with immunotherapy that had tumors classified as having high p-ERK demonstrated a significantly longer OS, with a hazard ratio (HR) of 0.23 compared to GBM patients having low p-ERK tumors that received immunotherapy (95% CI 0.079-0.69; P<0.0001, log-rank test; FIG. 2a). In this regard, the median OS of GBM patients with high p-ERK tumors was 13.89 months (422.5 days) compared to 2.72 months (83 days) for patients whose tumors had low p-ERK cell density. Conditional inference trees for cut-point optimization were also used to explore the interaction between p-ERK and anti-PD-1 therapy with respect to OS by utilizing the cohort that received PD-1 blockade. The optimal cut-point value of p-ERK cell density based on OS led to a value (3171 cells/mm²) of p-ERK high vs p-ERK low across patients that is close to the median p-ERK value (3207 cells/mm²) we used to dichotomize p-ERK as high vs low (FIG. 11a). In contrast, p-ERK was not associated with survival in recurrent GBM patients that did not receive immunotherapy (FIG. 2a). For this control cohort, survival was calculated from the date of surgery that led to the recurrent tumor specimen used for IHC staining. In this cohort, the high p-ERK group that had a median OS of 6.57 months (200 days) versus 6.65 months (202.5 days) in the low p-ERK group (HR=1.06, 95% CI 0.52-2.14; P=0.86, log-rank test). Furthermore, patients with high p-ERK tumors treated with PD-1 blockade also exhibited prolonged survival compared to those patients with high p-ERK tumors who did not receive the immunotherapy (HR=0.41, 95% CI 0.17-0.96; P=0.014, log-rank test; FIG. 2a). Of note, as of the last analysis, three patients classified as high p-ERK were still alive and receiving the immunotherapy.

To further assess p-ERK staining as a predictive biomarker for PD-1 immunotherapy, we performed univariable and multivariable analyses using the Cox proportional hazard model to evaluate the association of p-ERK cell density on survival. To this end, we first tested the proportional hazards assumption for OS on this cohort, which confirmed that the Cox proportional hazards model analysis was appropriate for this analysis. The univariable Cox analysis revealed that p-ERK cell density was associated with increased OS in patients treated with PD-1 blockade (HR=0.18, 95% CI 0.07-0.48; P=0.001, Wald test), but not in patients that were not treated with immunotherapy (HR=1.13, 95% CI 0.51-2.49; P=0.759, Wald test; FIG. 2b). We also investigated whether age, Karnofsky performance score (KPS), IDH mutations, MGMT promoter methylation, concurrent treatments, steroid use, tumor size or Ki67 labeling were associated with OS in the cohort of patients treated with PD-1 blockade, and found that age (HR=1.04, 95% CI 1.01-1.07; P=0.021) was weakly associated with shorter survival (FIG. 11b). Next, we investigated the influence of known prognostic factors for GBM patients such as age, MGMT promoter methylation status, and IDH mutations²⁷⁻²⁹ in our cohorts as part of a multivariable Cox model. This confirmed that ERK1/2 phosphorylation in patients treated with PD-1 inhibitors was associated with survival independently of these prognostic factors (HR=0.17, 95% CI 0.06-0.47; P=0.001, Wald test; FIG. 2b). The predictive value of p-ERK⁺ cells/mm² with respect to OS was also assessed using p-ERK by treatment interaction term (PD-1 blockade vs no immunotherapy). This analysis showed a significant association between p-ERK and OS (HR=0.18, 95% CI 0.06-0.56; P=0.003, Wald test). This supports the conclusion that elevated p-ERK is associated with OS prolongation only in the context of PD-1 inhibition.

To investigate the sensitivity and specificity of p-ERK as a predictive biomarker for response to PD-1 blockade, we generated ROC curves using 1-year OS as benchmark. In this context, the area under the curve (AUC) for the anti-PD-1 therapy cohort was 0.78 (95% CI 0.61-0.95) and 0.57 (95% CI 0.31-0.80) for the no immunotherapy cohort (FIG. 2c). Overall, these survival analyses indicate that high p-ERK is only associated with a survival benefit in the context of anti-PD-1 therapy, and therefore is predictive but not prognostic as a biomarker.

Next, we investigated the feasibility of using p-ERK staining for predicting response to PD-1 blockade through scoring by a neuropathologist who was blinded to outcomes and treatments. The pathologist designated the samples as high or low based on the staining pattern on the tumor regions. Whereas the pathologist's scoring showed a similar trend as the quantification of p-ERK (HR=0.35, 95% CI 0.095-1.3; P=0.0143, log-rank test; FIG. 11c), the association between p-ERK high tumors with OS was stronger when this biomarker was quantified. This analysis also suggests that computer-based quantification of p-ERK IHC might be more reproducible and reliable than scoring by pathologists.

We investigated whether elevated p-ERK cell density and its association with long-term survival following PD-1 blockade can occur irrespective of BRAF/PTPN11 activating mutations. Interestingly, patients that survived longer than 12 months from initiation of PD-1 blockade had elevated p-ERK⁺ cell density compared to patients that survived less than 12 months (P=0.01, Mann Whitney U test; FIG. 2d). Of the patients that survived more than 12 months, 5 patients had wild type BRAF and PTPN11, 4 had activating mutations in these genes, and 2 patients had unknown BRAF/PTPN11 mutational status. These results show that patients that experienced a longer OS in the context of anti-PD-1 therapy consistently exhibit high levels of p-ERK regardless of the genetic status of the BRAF/PTPN11 genes.

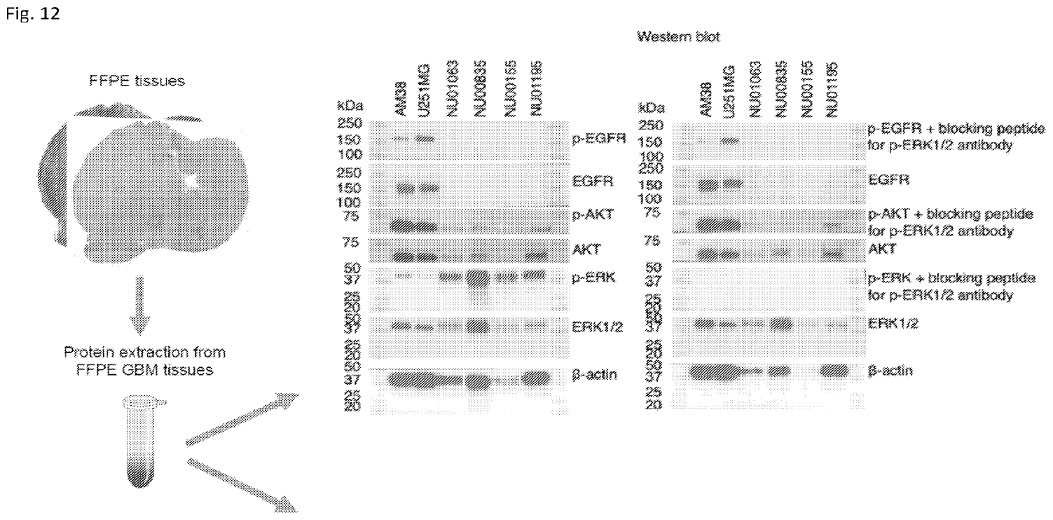

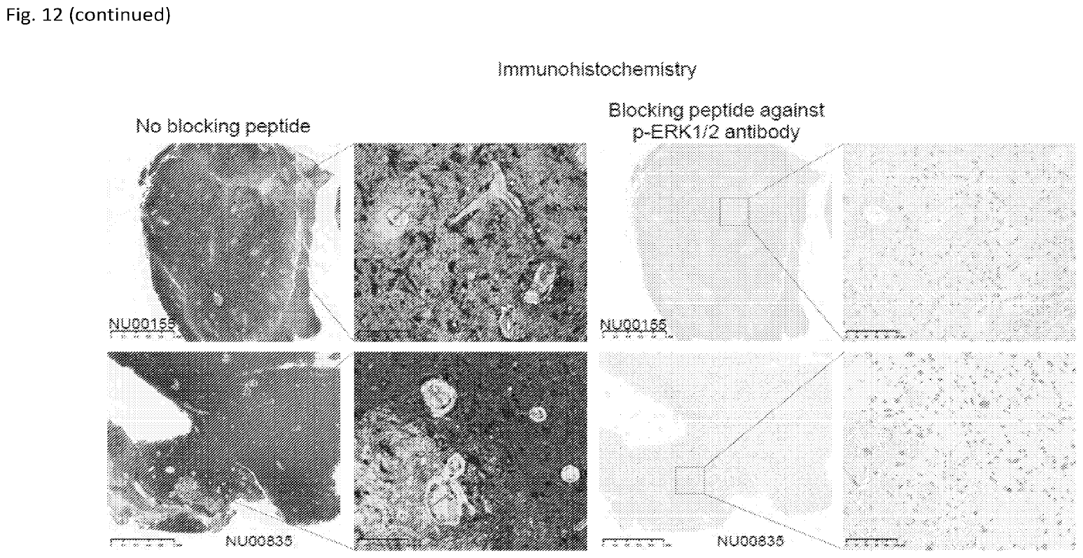

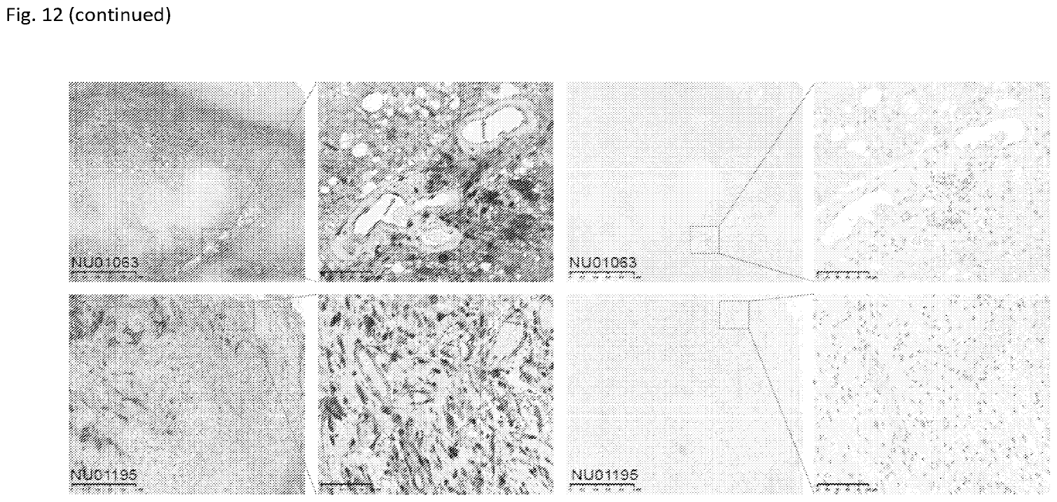

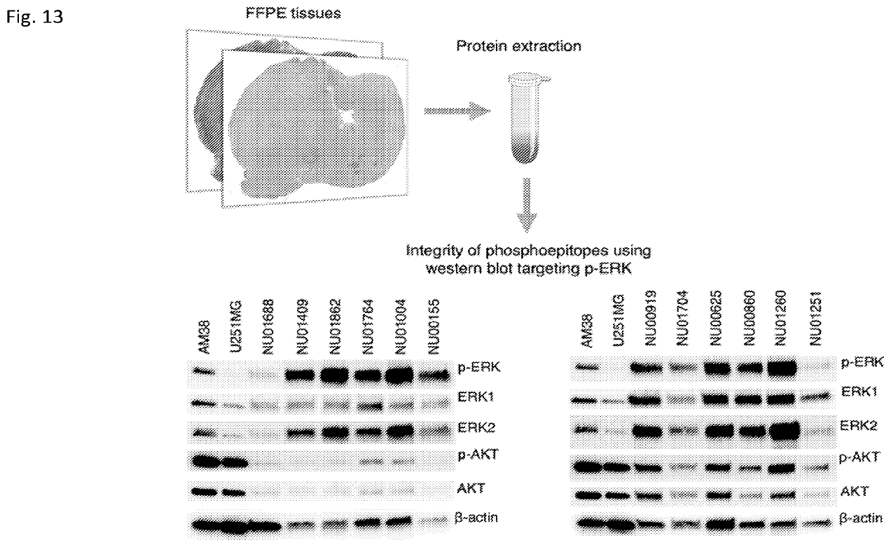

Assessment of phospho-epitope integrity and specificity of the IHC staining for p-ERK determination. Given that ischemic time can lead to degradation of phospho-epitopes such as p-ERK, and to rule our non-specific staining as potential confounders of our survival analysis relying on p-ERK IHC, we investigated the integrity and specificity of p-ERK staining in our samples. We performed a peptide competition assay by western blot and IHC and confirmed unequivocal specific staining of p-ERK in formalin-fixed paraffin-embedded (FFPE) GBM samples, ruling out any kind of cross-reactivity by the p-ERK antibody (FIG. 12). Additionally, we evaluated the preservation of the p-ERK in a set of unstained slides from FFPE GBM samples used that were used for the survival analysis, and using western blot we detected the expected band for p-ERK on these samples, confirming the integrity of the p-ERK phospho-epitope (FIG. 7).

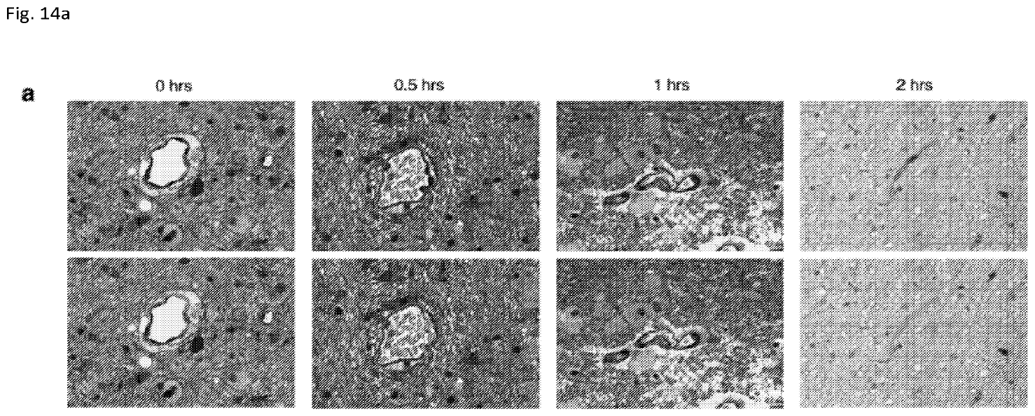

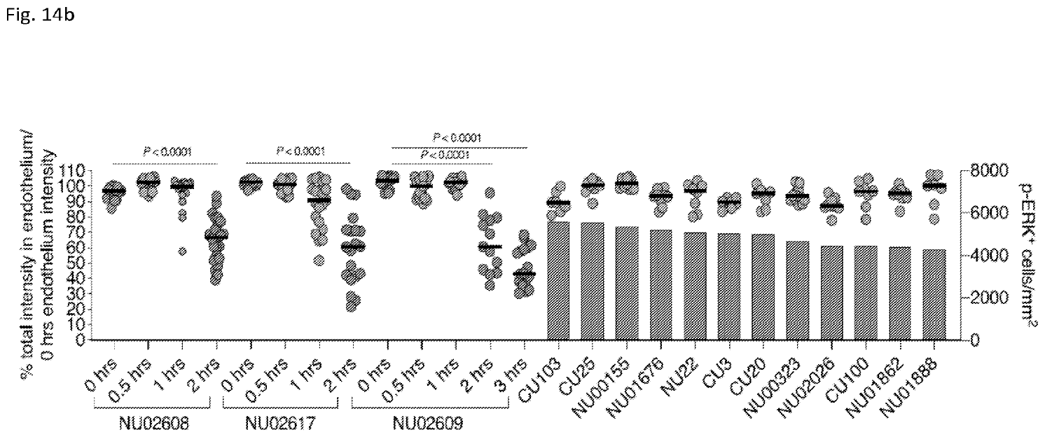

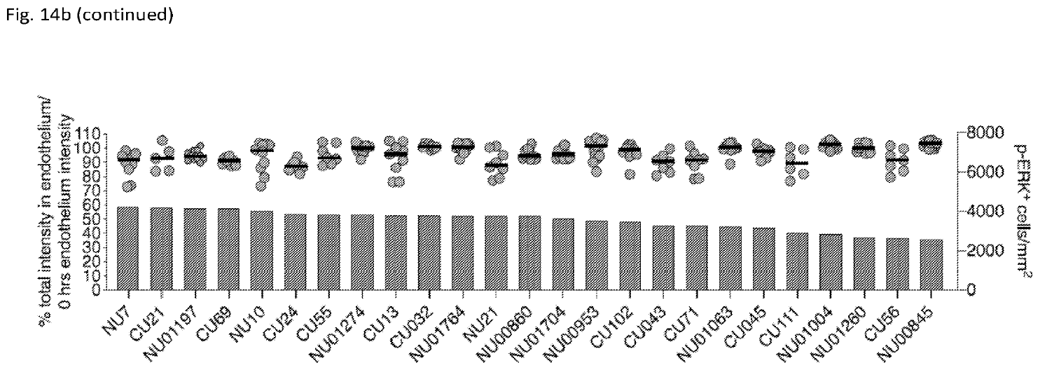

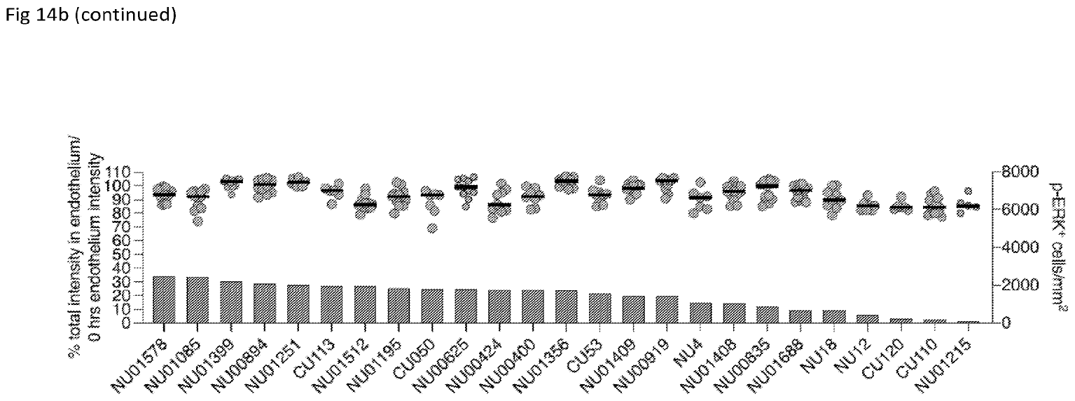

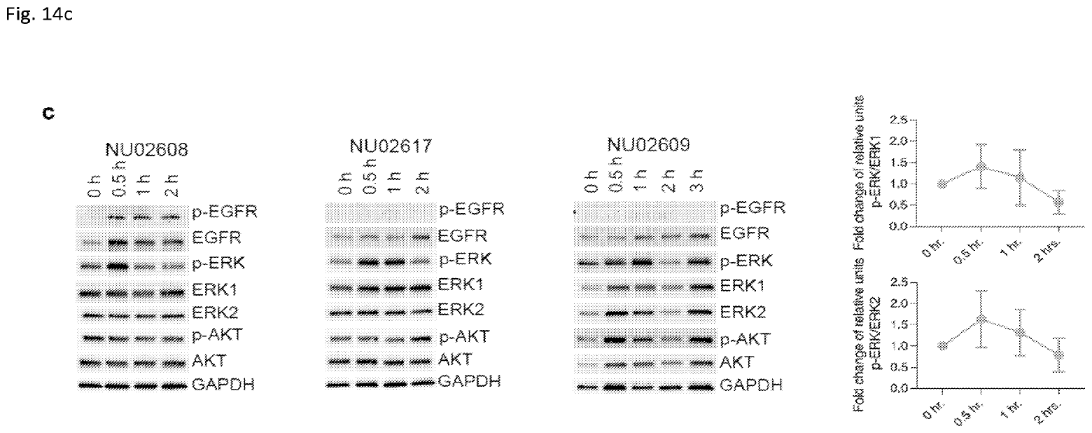

Given that preanalytical variables such as delay in fixation time can affect phosphorylated proteins^{30,31}, we studied the degradation dynamics of p-ERK in GBM samples after different ischemic times. We obtained fresh GBM tumor specimens and fixed them at different time intervals following surgical resection (0 hr., 0.5 hr., 1 hr., 2 hrs.). Then, we stained these samples against p-ERK using the same antibody dilution. We analyzed endothelial cells as a common denominator across samples as p-ERK is strongly expressed by these cells^{32,33}. We quantified and compared the intensity of endothelial p-ERK staining for each tumor sample fixed at different ischemic time points and for tumor samples in the discovery cohort (FIG. 14a). We found that the intensity of endothelial p-ERK in GBM samples from our discovery cohort used for the survival analysis remains relatively stable, without a significant decrease relative to 0 minutes of ischemia time, and for up to 1 hour of ischemic time. Endothelial p-ERK staining exhibits degradation starting at 2 hrs. time point (FIG. 14b). Additionally, we performed western blot evaluating phospho-ERK, phospho-AKT, and phospho-EGFR in tumor samples subjected to different ischemic times (FIG. 14c). Collectively, these results show that p-ERK epitope is preserved for up to 1 hr. of ischemic time, and that the samples stained and on the samples from our discovery cohort analyzed, p-ERK is detectable and the staining observed is specific for this phospho-protein.

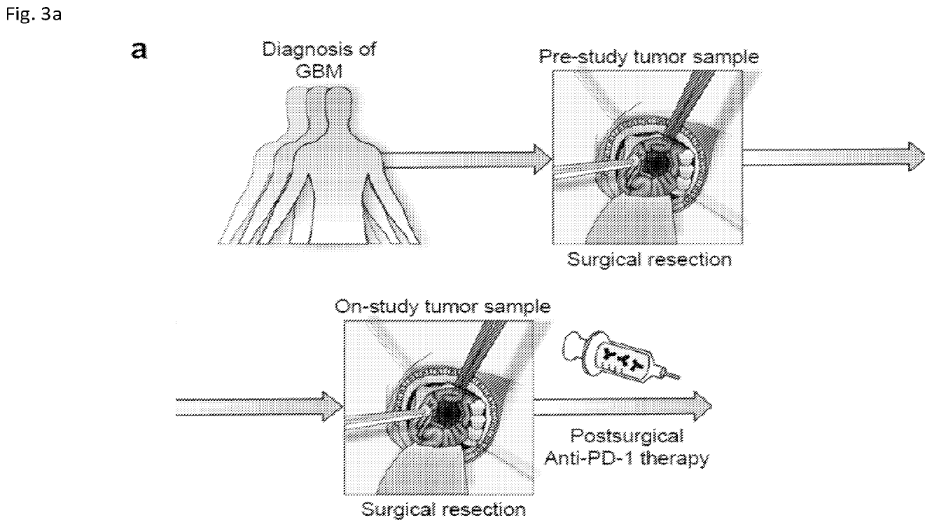

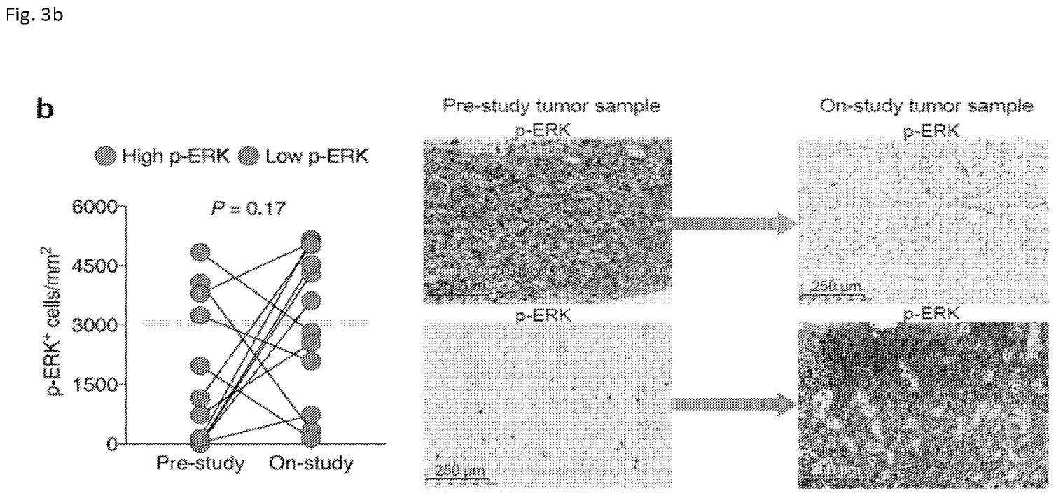

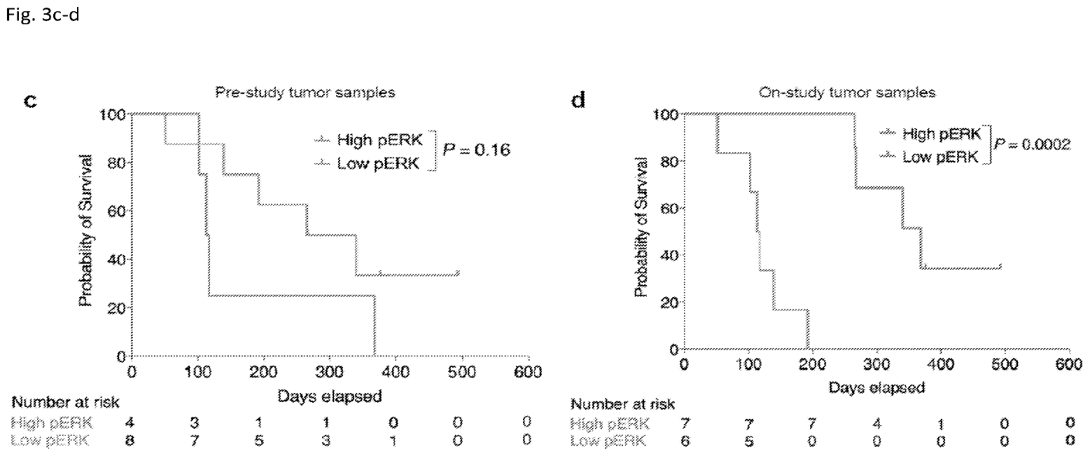

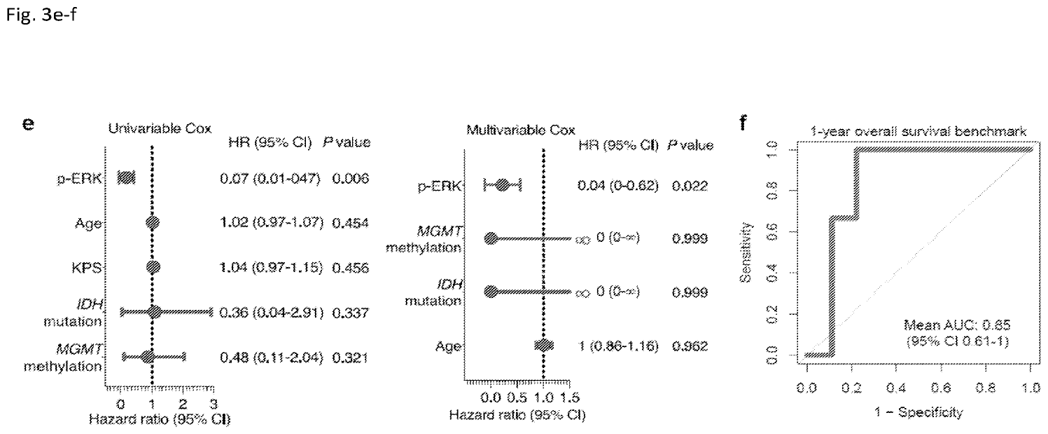

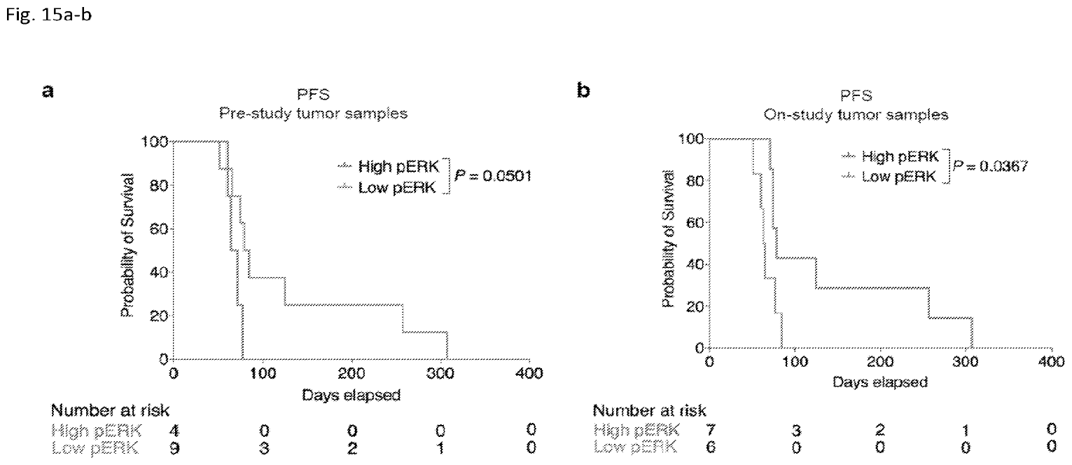

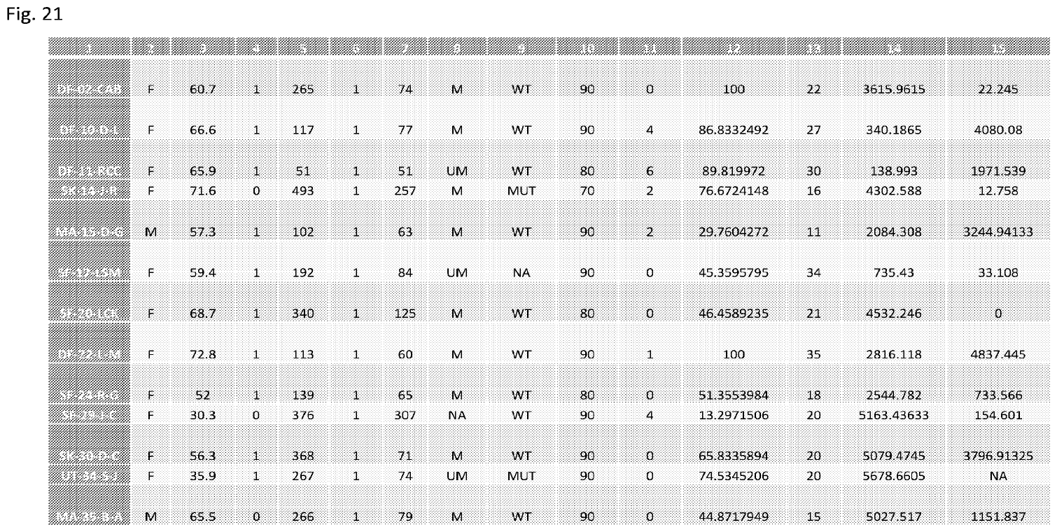

p-ERK determined on samples acquired shortly before initiation of PD-1 blockade better predict response in recurrent GBM. Tumor cell phenotype and the associated microenvironment can change over time, particularly between newly diagnosed GBM and recurrent disease^{2,34}. To investigate whether the timing of tissue used for p-ERK determination relative to initiation of PD-1 blockade influences the predictive properties of this biomarker, we used paired samples obtained at different time points prior to initiation of PD-1 blockade from the Cloughesy T F. et al. clinical trial¹⁶ (NCT02852655), which included an arm that underwent adjuvant PD-1 administration (analyzed samples were naïve to PD-1 blockade) (FIG. 21). We compared p-ERK from GBM specimens obtained during the surgery that preceded the enrollment on this trial (pre-study samples) versus samples from the same patients obtained during surgery at recurrence, as part of the trial, weeks before initiating treatment with PD-1 inhibitors (on-study tumor samples) (FIG. 3a). This cohort also served as an independent validation set where tumor samples and clinical outcomes were collected prospectively. For this validation cohort, the designation of high vs low p-ERK gliomas was done using the same quantification method and cut-point value (3207 cells/mm²) used to partition groups in the discovery cohort. Though we found no systematic overall change of p-ERK high vs low designation between the two timepoints (P=0.17, Wilcoxon rank test), the p-ERK cell density greatly fluctuated between pre- and on-study time points for most of the tumors, including the majority of cases that were initially designated as high p-ERK that became low p-ERK (91.6%) and 62.5% that were initially designated as low that became high p-ERK in the group of on-study samples (FIG. 3b). Next, we performed survival analysis to evaluate the performance of pre-study samples and on-study samples to predict OS following anti-PD-1 therapy. Based on the staining of pre-study samples, patients from low p-ERK group exhibited marginal longer progression-free survival (PFS) compared to the high p-ERK group (P=0.0501, Log-rank test; FIG. 15a), and no significant differences in OS (P=0.16, Log-rank test; FIG. 3c). In contrast, when using the on-study samples for staining and analysis, the high p-ERK group exhibited a longer PFS (P=0.0367, Log-rank test; FIG. 15b) as well as longer OS (P=0.0002, Log-rank test; FIG. 3d) compared to the low p-ERK patients. The median OS of GBM patients with high p-ERK tumors was 12.09 months (368 days) compared to 3.78 months (115 days) for patients whose tumors had low p-ERK activation. These results suggest that tumors acquired shortly before PD-1 blockade initiation are more reliable to predict response of recurrent GBM patients to PD-1 blockade. To further validate our initial observations, we performed the Cox proportional hazard model to test the association of p-ERK cell density with survival using the on-study tumor samples. In this independent GBM cohort, p-ERK cell density was associated with OS in the univariate (HR=0.07, 95% CI 0.01-0.47; P=0.006, Wald test) and multivariable analysis (HR=0.04, 95% CI 0-0.62; P=0.022, Wald test; FIG. 3e). We also employed ROC curves to determine the ability of p-ERK cell density to distinguish between recurrent GBM patients living more than 1 year in the validation cohort. The mean AUC was 0.85 (95% CI 0.61-1) further demonstrating the ability of p-ERK to discern which GBM patients would be appropriate candidates for anti-PD-1 therapy (FIG. 3f).

In sum, these results emphasize the predictive power of p-ERK from GBM samples acquired close to initiation of PD-1 blockade. Furthermore, these results validate our initial observations that high p-ERK is associated with prolonged survival for patients that were treated with adjuvant PD-1 blockade.

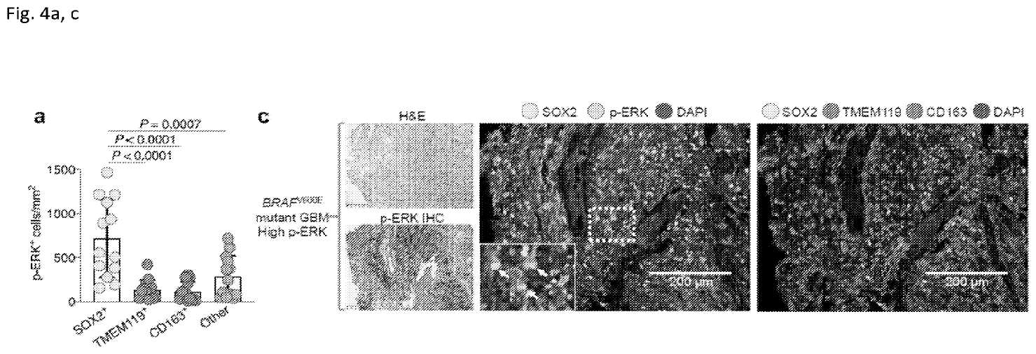

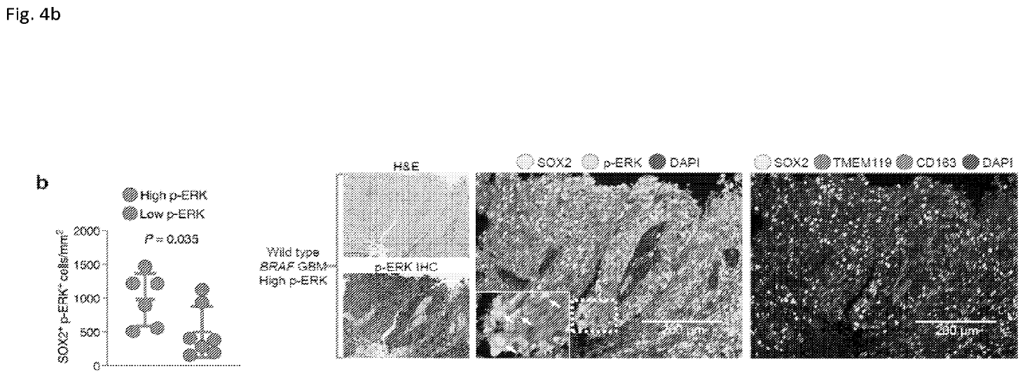

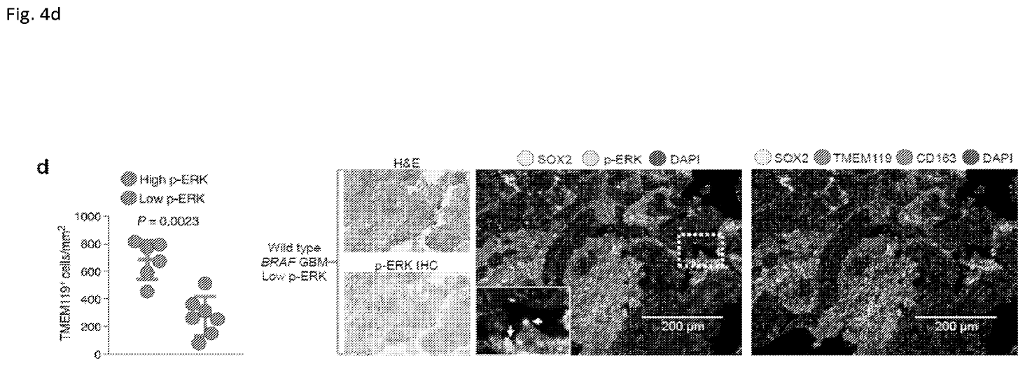

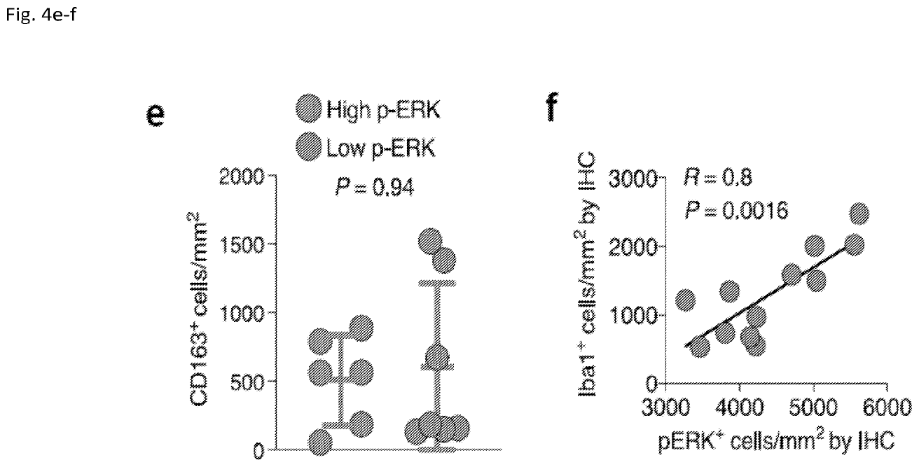

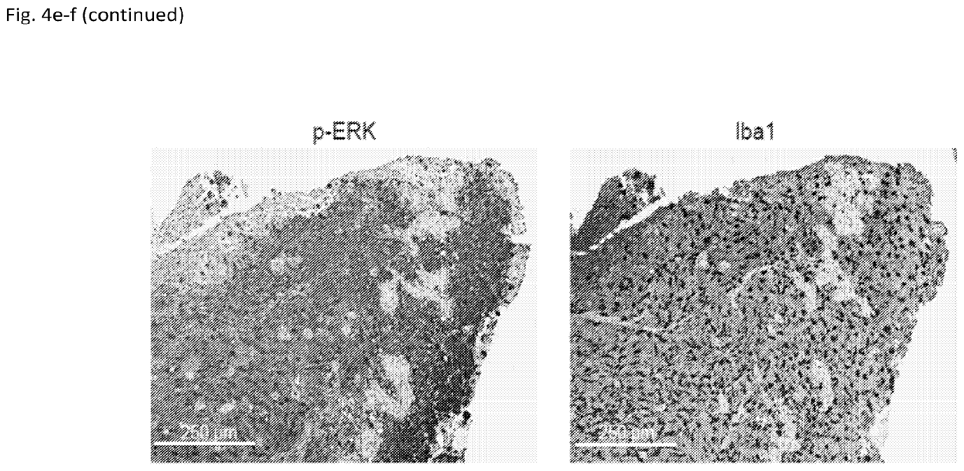

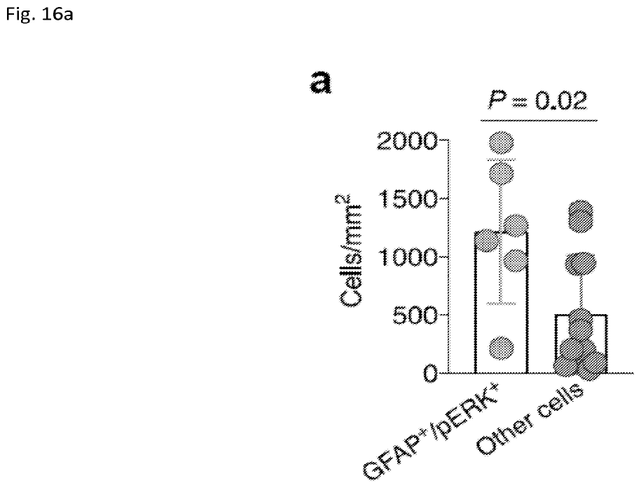

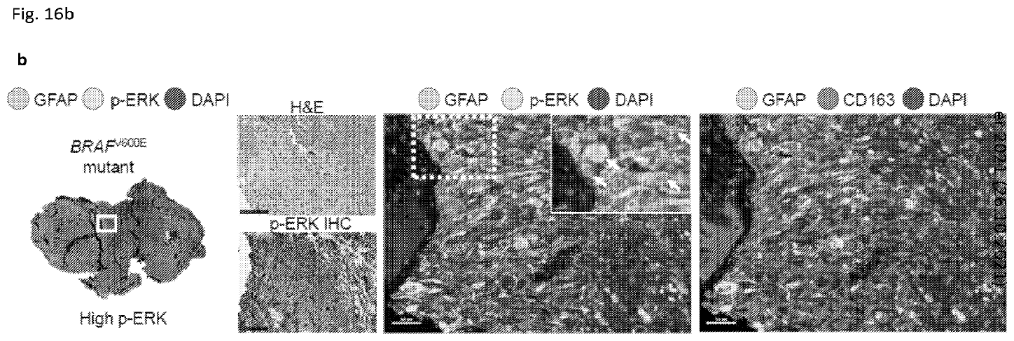

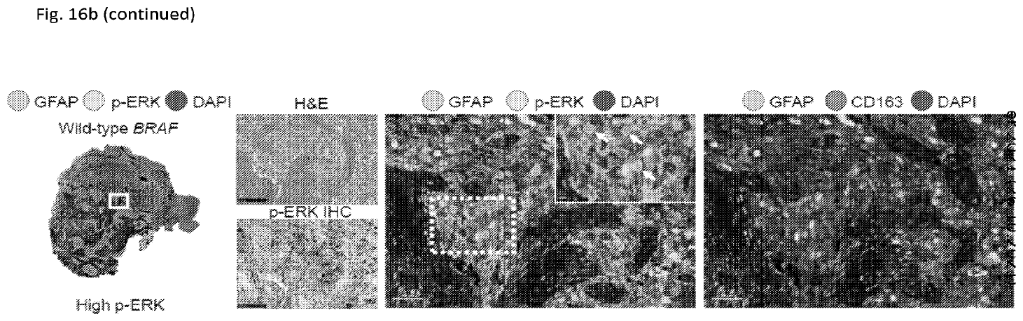

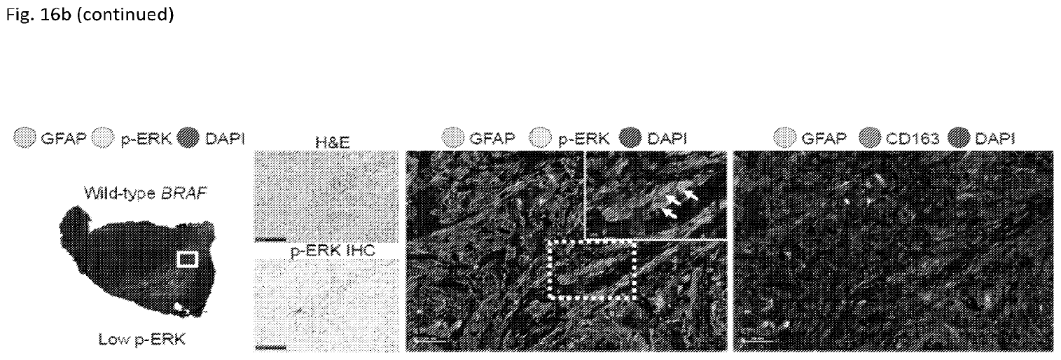

ERK1/2 phosphorylation localizes in tumor cells and is associated with tumor-infiltrating myeloid cells and microglia. To investigate which cell populations contribute to p-ERK staining in GBM, we performed multiplex immunofluorescence analysis in GBM samples evaluating p-ERK and the predominate cellular populations in the tumor microenvironment, including SOX2 (tumor marker³⁵), TMEM119 (microglial marker^{34,36,37}), CD163 (myeloid cell marker³⁷⁻³⁹), and DAPI. By quantifying these cell populations, we found that the phosphorylation of ERK1/2 was detected predominantly in SOX2⁺ cells relative to TMEM119⁺ cells (P<0.0001, one-way ANOVA), CD163⁺ cells (P<0.0001, one-way ANOVA), and other cells (SOX2⁻ TMEM119⁻ CD163⁻ cells, P=0.0007, one-way ANOVA; FIG. 4a). Multiplex staining utilizing GFAP to label tumor cells also confirmed that most p-ERK staining was predominantly associated with tumor cells/astrocytes (FIGS. 16a and b). We also found higher numbers of SOX2⁺ p-ERK⁺ cells in high p-ERK gliomas compared to low p-ERK gliomas (P=0.035, Mann Whitney U test; FIG. 4b). As representative examples, we present a BRAF^{V600E} mutated GBM and a tumor that was wild type for BRAF/PTPN11, both of which had high levels of phosphorylation of ERK1/2 expressed by SOX2⁺ cells (FIG. 4c). In contrast, in a recurrent GBM classified as having low levels of ERK1/2 activation by IHC, the majority of the SOX2⁺ cells were negative for p-ERK (FIG. 4c). These results show that the majority of p-ERK⁺ cells in gliomas are tumor cells with smaller contributions to p-ERK from immune and stromal cells.

Considering the abundance of myeloid cells in the GBM microenvironment^{39,40}, and their implication in the response and resistance to immune checkpoint inhibitors in GBM^{7,41-43}, we analyzed whether p-ERK cell density is associated with the infiltration of these immune cells in GBM samples. We found that the numbers of TMEM119⁺ cells were elevated in GBM with high p-ERK relative to the low p-ERK tumors (P=0.0023, Mann Whitney U test; FIG. 4d). Yet no difference was found in the numbers of glioma-infiltrating CD163⁺ cells between gliomas with high vs low p-ERK levels (P=0.94, Mann Whitney U test; FIG. 4e). We also stained glioma samples with the macrophage/microglia cell marker Iba1 by IHC and evaluated its association with p-ERK cell density. We found that p-ERK cell density derived from the IHC quantification (which was used to predict response to PD-1 blockade), correlated with the Iba1⁺ cell density across specimens (R=0.8, P=0.0016, Pearson r correlation; FIG. 4f).

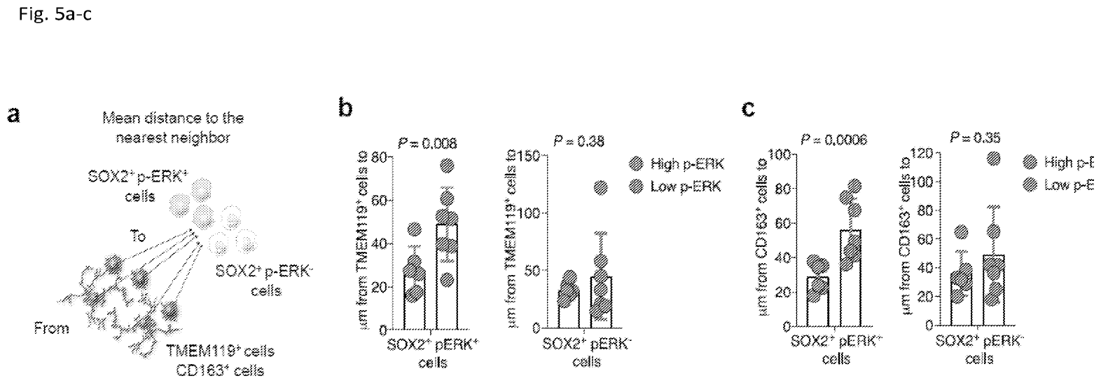

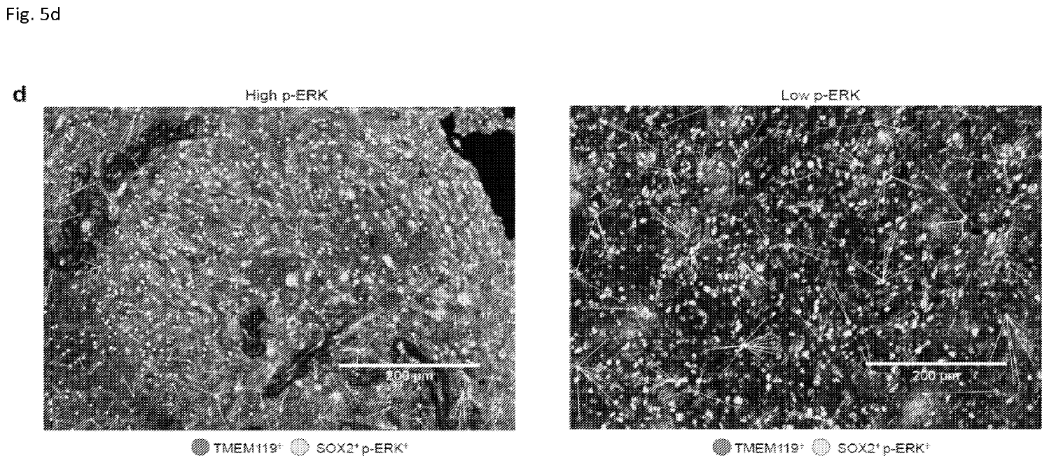

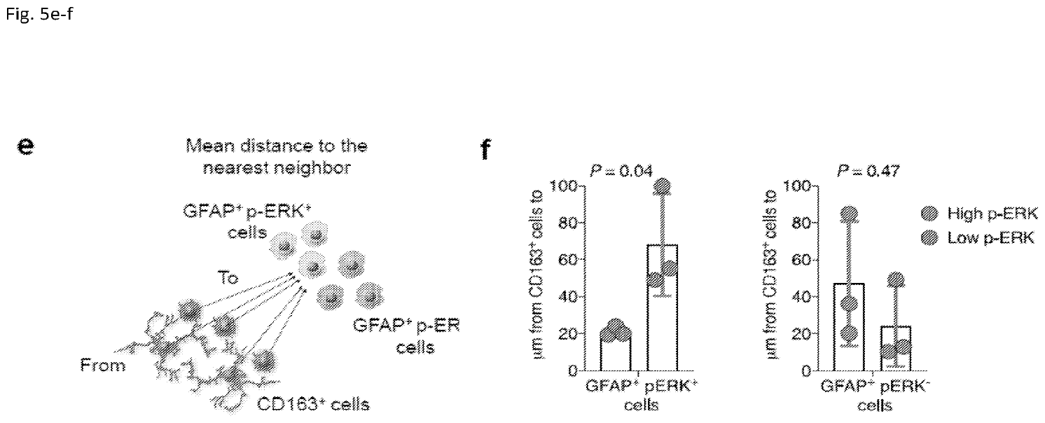

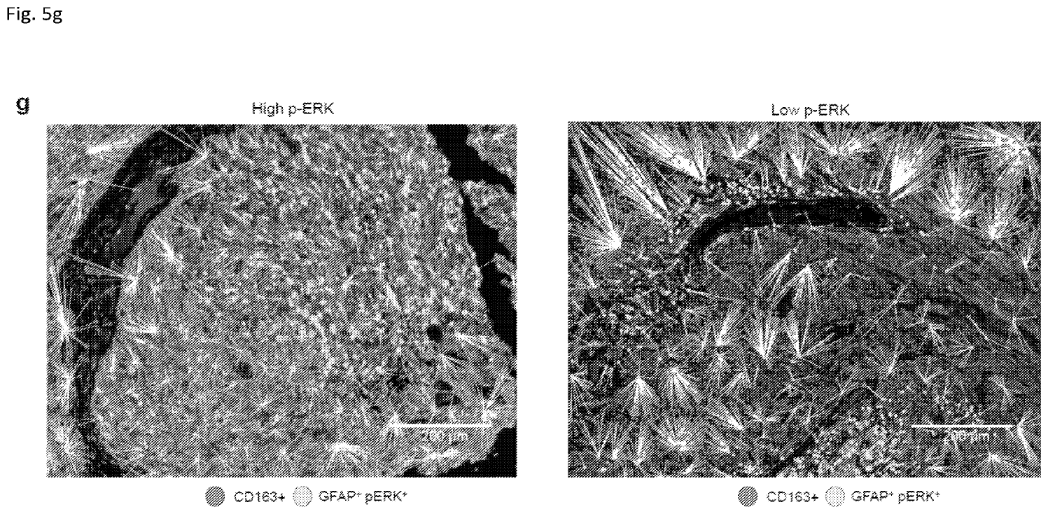

Next, we performed spatial analyses to further investigate the interaction between SOX2⁺ p-ERK⁺ cells and glioma-infiltrating myeloid cells. We calculated the distances between TMEM119⁺ and CD163⁺ cells to SOX2⁺ p-ERK⁺ cells (FIG. 5a). We found that TMEM119⁺ cells were closer to SOX2⁺ p-ERK⁺ cells in high p-ERK tumors compared to low p-ERK tumors (P=0.008, unpaired t test; FIG. 5b). Conversely, we did not find differences in distances between TMEM119⁺ cells and SOX2⁺ p-ERK⁻ cells between high and low p-ERK groups (P=0.38; unpaired t test; FIG. 5b). Similarly, the distances between CD163⁺ cells to SOX2⁺ p-ERK⁺ were shorter in tumors with high p-ERK than those with low p-ERK (P=0.0006, unpaired t test; FIG. 5c), yet no difference was noted in the distance between CD163⁺ cells to SOX2⁺ p-ERK⁻ cells between high and low p-ERK tumors (P=0.35; unpaired t test; FIG. 5c). Representative images for this spatial analysis are presented on FIG. 5d. Similarly, when using GFAP⁺ to label tumor/astrocytes to measure the distance between tumor cells and myeloid cells (FIG. 5e), we found that the distance from CD163⁺ cells to GFAP⁺ p-ERK⁺ was shorter for tumors with high p-ERK than those with low p-ERK (P=0.04, unpaired t test), whereas no differences in these distances were noted for GFAP⁺ pERK⁻ cells (P=0.47, unpaired t test; FIG. 5f). Representative images for this analysis are presented on FIG. 5g. These results suggest that gliomas with an elevated number of p-ERK⁺ cells are associated with high infiltration of myeloid cells. Compared to other solid tumors in which there is a separation between immune cells and tumor regions⁴⁴, the elevated number of p-ERK⁺ tumor cells in close proximity to glioma-infiltrating myeloid cells suggests a high degree of mixing between these cell populations.

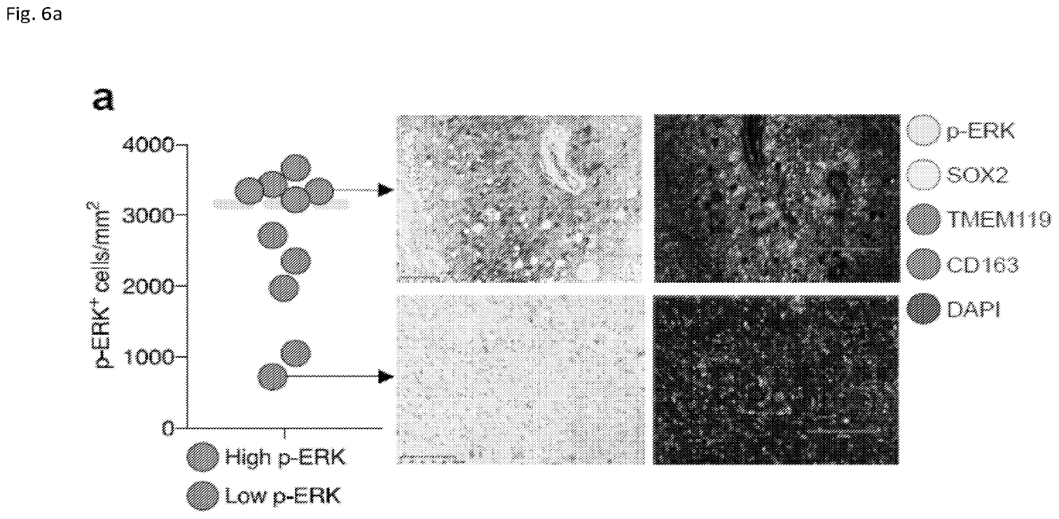

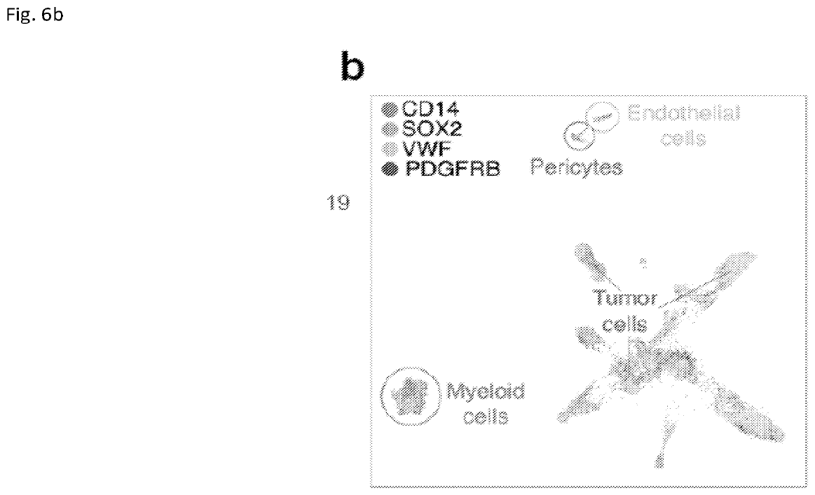

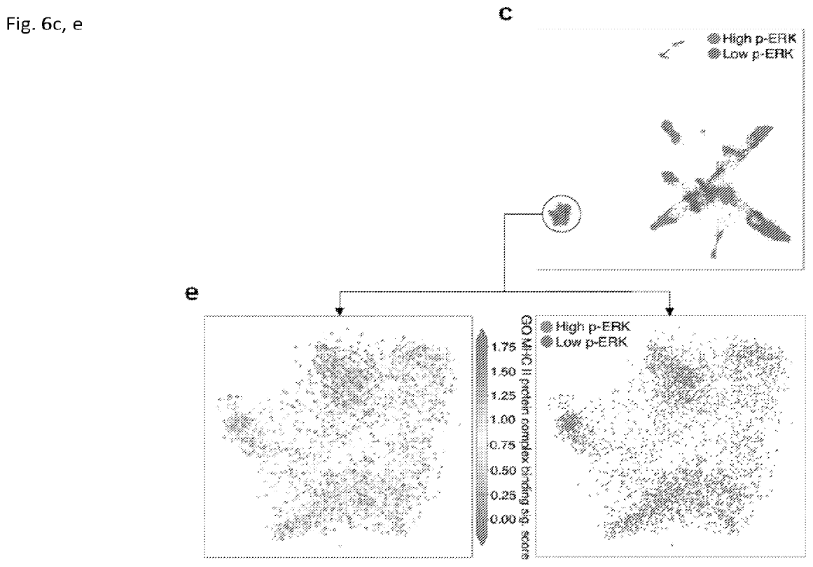

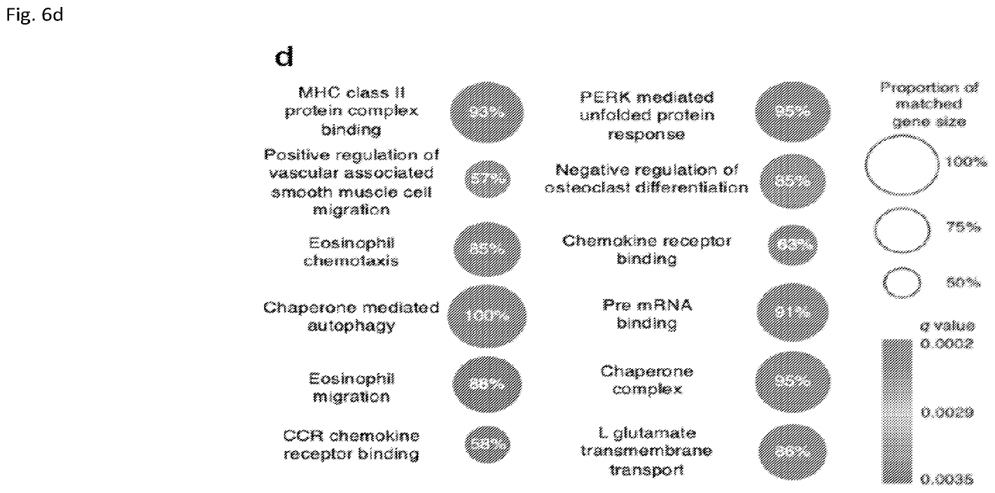

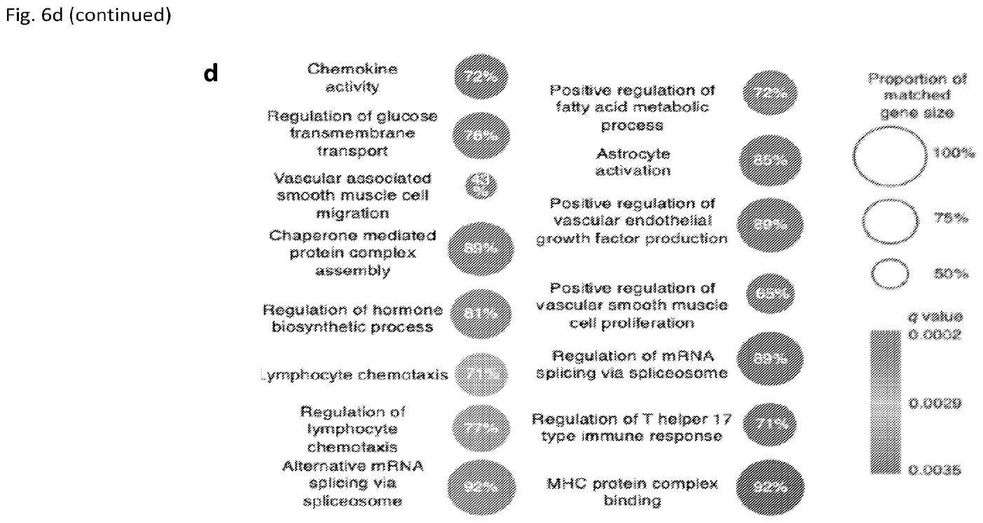

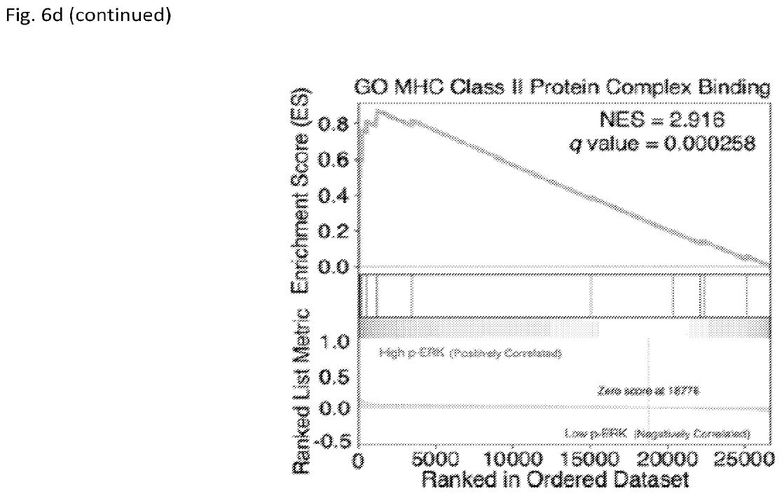

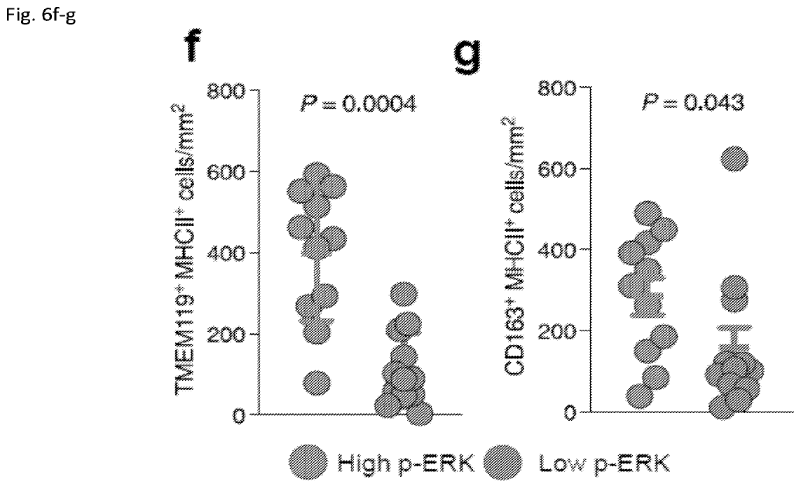

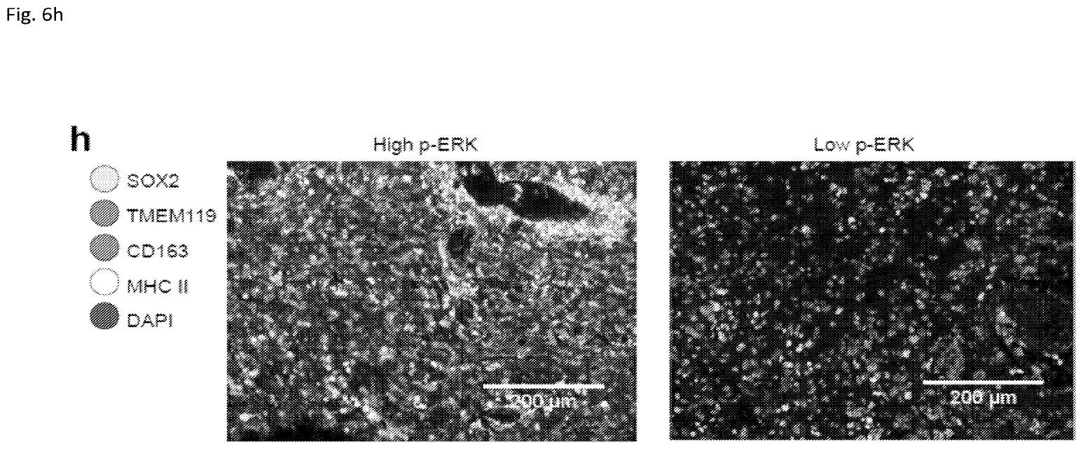

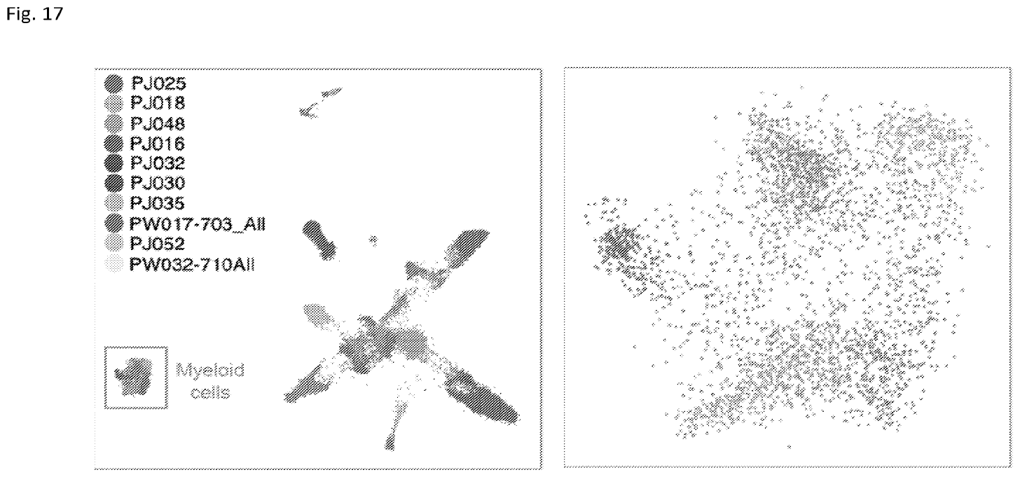

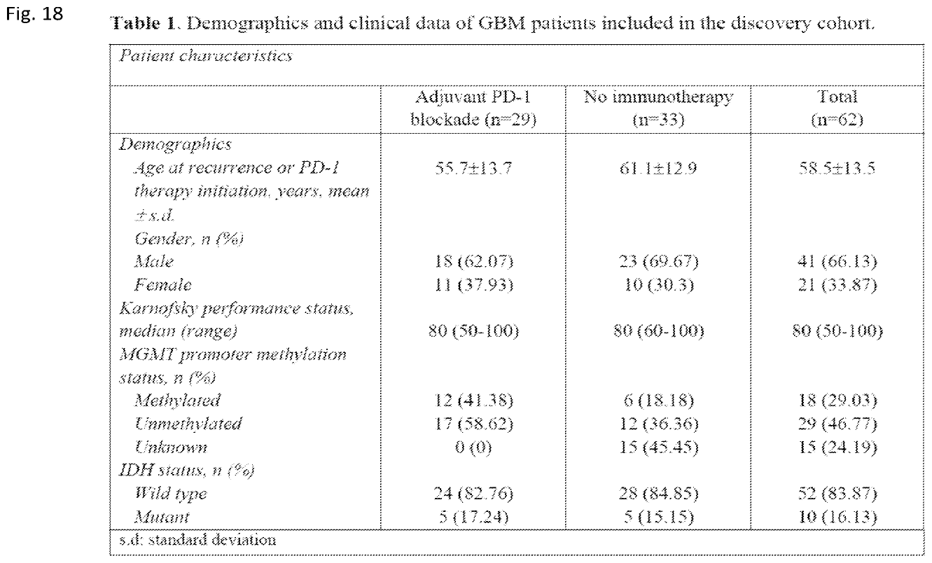

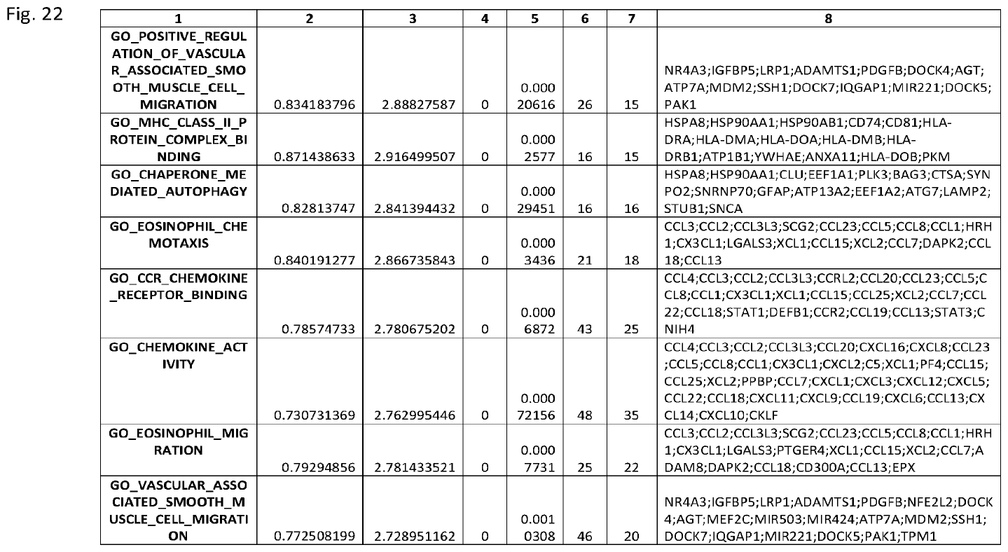

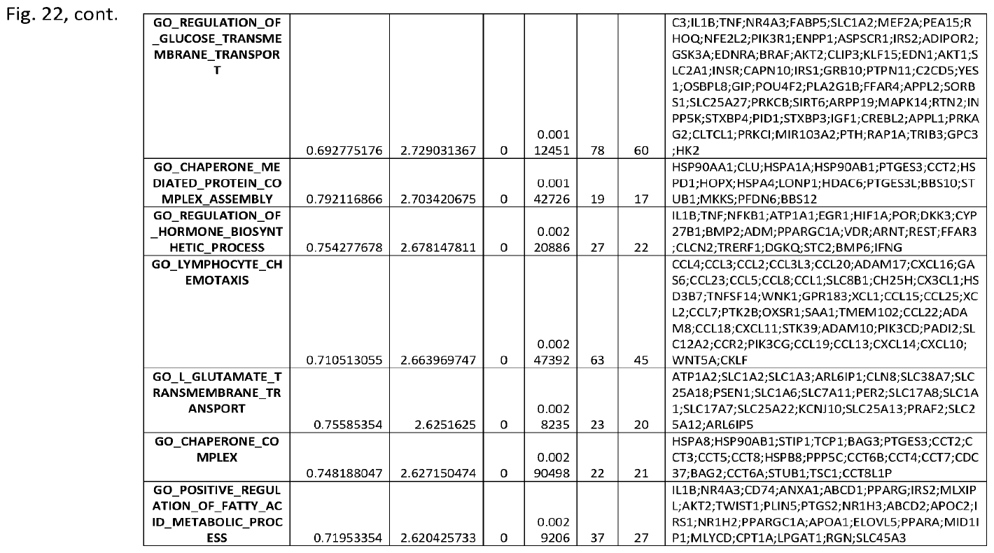

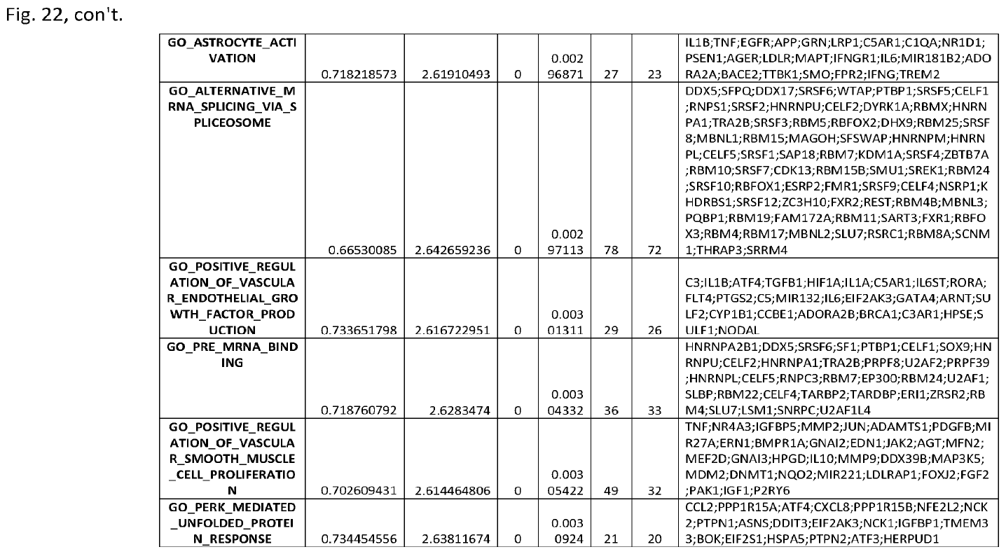

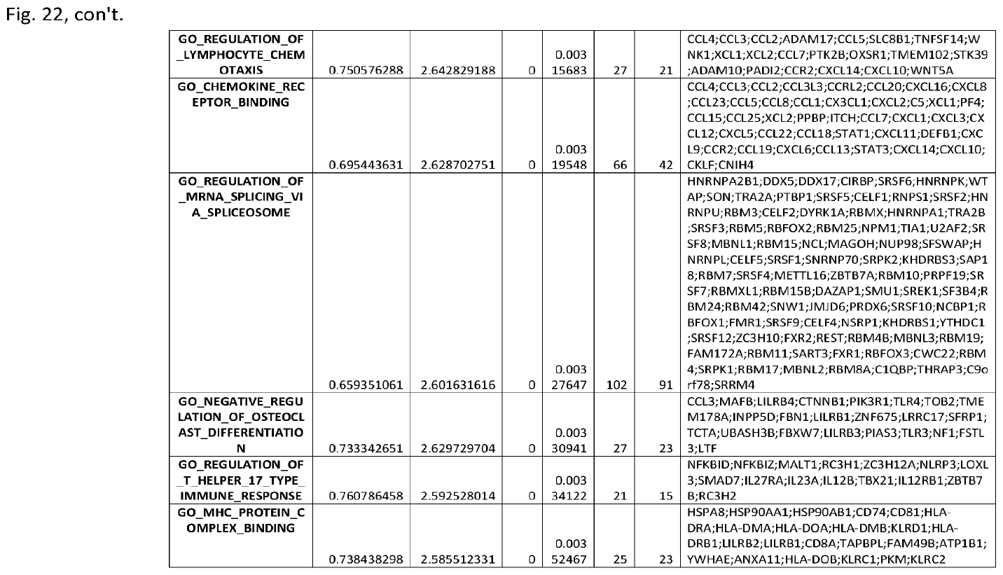

Tumor-infiltrating myeloid cells of GBM with elevated p-ERK1/2 have a distinct phenotype with upregulation of MHC class II. Given the myeloid cell infiltration we observed to be associated with ERK1/2 activation in tumor cells, we investigated the phenotype of these myeloid cells using scRNA-seq. We analyzed 21,326 cells from ten GBM specimens³⁵ that were paired with p-ERK IHC staining. p-ERK cell density was quantified using the same methodology as the discovery and validation GBM cohort, and we used the same cut-point value to designate tumors as high vs low p-ERK, resulting in 5 p-ERK high tumors and 5 p-ERK low tumors (FIG. 6a). Next, we performed unsupervised clustering of all cells without employing cell type markers (FIG. 17). Subsequently, we annotated the clusters based on the expression of validated cell type markers used for single-cell transcriptomics for tumor cells (SOX2), myeloid cells (CD14), endothelial cells (VWF), and pericytes (PDGFR B)³⁵. Whereas tumor cells clustered into several sample-dependent groups, the myeloid cell compartment, pericytes, and endothelium derived from all GBM samples clustered uniformly (FIG. 6b for cell phenotype, FIG. 6c for high vs low p-ERK designation from IHC, and FIG. 17 for per tumor sample UMAP plot). We focused our analysis on myeloid cells given their increased infiltration in tumors with elevated p-ERK, their relative abundance and their known immunomodulatory roles in the context of immune checkpoint blockade in GBM^{7,41-43}. To investigate the transcriptional differences within the myeloid cell population between high and low p-ERK tumors, we performed gene set enrichment analysis (GSEA⁴⁵) with the gene ontology (GO) set collection. With this analysis, 27 pathways were significantly enriched in the myeloid cells derived from high p-ERK tumors (false discovery rate (FDR), q<0.05; FIG. 6d and FIG. 22. Interestingly, the top gene set enriched in the myeloid cells infiltrating high p-ERK tumors was the GO term “MHC class II protein complex binding” (NES=2.916, q=0.000258; FIGS. 6d and e), among several other GO themes related to lymphocyte chemotaxis, chemokines and antigen presentation (FIG. 6d). In contrast, we did not find significant gene sets enriched in myeloid cells from low p-ERK tumors. Given the enrichment of the “MHC class II protein complex binding” GO term in myeloid cells from high p-ERK GBMs, we investigated whether these myeloid cells also express the MHC class II (MHC II) molecule. We employed multiplex immunofluorescence staining evaluating SOX2 (tumor cell marker), CD163 (myeloid cell marker), TMEM119 (microglial marker), MHC II, and DAPI. This analysis revealed that TMEM119⁺ cells from high p-ERK tumors had elevation of MHC II protein expression relative to tumors with low p-ERK (P=0.0004, Mann Whitney U test; FIG. 6f). We also observed a similar trend for MHC II protein expression in tumor-infiltrating CD163⁺ myeloid cells of high p-ERK tumors relative to tumors with low p-ERK (P=0.043, Mann Whitney U test; FIG. 6g). Representative images for this analysis are presented on FIG. 6h. Collectively, these results suggest that tumors with elevated activation of ERK1/2 are characterized by a particular microenvironment in which myeloid cells, mostly microglia, express MHC II and a gene signature related to this antigen-presenting molecule.

Discussion

Our study supports previous observations that only a fraction of GBM patients treated with PD-1 blockade exhibit radiographic responses and a survival benefit. Although a recent clinical trial of PD-1 blockade in recurrent GBM failed to show an OS increase, the study reported positive responses in 7.8% of patients based on radiographic criteria, with prolonged durability of response as typically seen with PD-1 inhibitors in other solid tumors⁵. Given that the majority of patients discontinued the immunotherapy due to tumoral progression assessed by radiographic response, which can be related to pseudoprogression⁵, the percentage of GBM patients who may benefit from PD-1 blockade could be higher. Moreover, recent studies report encouraging results for the efficacy of PD-1 blockade in the neoadjuvant setting^{16,46}.

Predictive biomarkers of response to PD-1 inhibitors in other cancers include PD-L1 protein expression, high tumor mutational burden (TMB), mismatch repair (MMR) protein deficiency⁴⁷⁻⁴⁹, and the status of the tumor immune microenvironment⁵⁰. Studies have reported efficacy of immune checkpoint inhibitors in patients with GBMs harboring germline mutations in MMR pathways and the POLE gene^{15,51,52}. However, these biomarkers (high TMB, MSI-high, POLE mutations, tumor-infiltrating lymphocytes, and PD-L1 expression) are not informative for the majority of GBMs⁵⁻⁷. MMR-deficient hypermutant tumors that arise as a result of treatment with temozolomide do not seem to benefit from immune checkpoint inhibitors⁵³⁻⁵⁵. Indeed, recent evidence indicates that mutations derived from chemotherapy-induced MMR deficiency tend to be subclonal and less immunogenic^{55,56}. Thus, to date there are no reliable markers for response to PD-1 blockade for GBM patients.

We previously reported that recurrent GBMs responsive to PD-1 blockade were enriched in activating mutations in BRAF and PTPN11. Nevertheless, these mutations were only present in 2-3% of GBM (˜30% of responsive patients)¹⁴. In this study, we report that patients that respond to PD-1 blockade exhibit activation of the ERK cascade (phosphorylation of ERK1/2), including those that do not have mutations in BRAF/PTPN11. The median OS in recurrent GBM is approximately 5-9 months, with resectable tumors closer to 9 months⁵⁷⁻⁶⁰. We observed that 62.5% of the GBM patients in the high p-ERK group were alive at 1 year after initiation of the immunotherapy compared to 0% of patients in the low p-ERK group. This survival benefit is unlikely to be explained by survivorship bias from resectable patients as all underwent surgery, and it seems to be independent of other prognostic clinical and molecular variables. These results are encouraging, as the effect was validated on an independent cohort in which data was collected prospectively as part of a clinical trial. Nevertheless, adoption of p-ERK as a predictive biomarker will require prospective validation in additional clinical trials.

Our study identified robust differences in the microenvironment associated with p-ERK/MAPK signaling in GBM. Particularly, an increased number of tumor-infiltrating myeloid cells and microglia in proximity to p-ERK⁺ tumor cells in GBM that have elevated p-ERK, which responded to PD-1 blockade. Similarly, we showed that development of mouse transgenic gliomas in the absence of CD8⁺ T-cells leads to elevated intratumoral p-Erk1/2, and that this signaling correlates with a robust increase tumor infiltration by Cd11b⁺ and Iba1⁺ myeloid cells, and a proinflammatory tumor microenvironment⁶¹.

While some studies have characterized immunosuppressive glioma-infiltrating myeloid cells^{42,62,63}, some others have described antitumoral myeloid cells in the setting of cancer immunotherapy, including in glioma^{43,64,65}. In a recent preclinical study, transgenic gliomas in CD8a KO mice, which are depleted for CD8⁺ T-cells had prolongation of survival after PD-1 blockade 43 Of note, murine gliomas with genetic ablation of CD8a had increased percentage of proinflammatory CD11b⁺ MHC 11 cells and peripheral expansion of CD4⁺ T cells compared to their wild-type CD8a counterparts. This evidence highlights the possible direct contribution of myeloid cells and microglia to response to PD-1 blockade^{43,61}.

Glioma-infiltrating myeloid cells exhibit a range of phenotypes from immunosuppressive to pro-inflammatory. Our investigation suggests that infiltrating myeloid cells of high p-ERK tumors might have higher antigen presentation capabilities via MHC II. Indeed, scRNA-seq from myeloid cells from tumors with high p-ERK showed enrichment of genes related to MHC II, a finding that we confirmed with multiplex immunofluorescence for MHC II. In addition, high p-ERK tumors were also enriched in GO terms related to lymphocyte chemotaxis, chemokines and antigen presentation. A recent study suggested that recurrent GBMs with low mutational burden have enrichment of inflammatory gene signatures including a MHC II gene set, and longer survival after immunotherapy⁶⁶. These findings concur with a recent study of metastatic melanomas, in which increased expression of MHC II-associated genes was associated with response to anti-PD-1 immunotherapy⁶⁷. In this regard, antigen presentation by MHC II to CD4⁺ T cells has been suggested to be necessary to induce an effective anti-tumoral response during immune checkpoint inhibition⁶⁸. On the other hand, in GBM patients treated with neoadjuvant PD-1 blockade, peripheral CD4⁺ T cells had elevated expression of CD152 (CTLA4) and CD127 (IL7-Rα) but downregulation of PD-1¹⁶. These findings together with our data suggest an interplay between MHC II-mediated antigen presentation with peripheral activated memory CD4⁺ T cells shown to increase after anti-PD-1 therapy in recurrent GBM patients.

Our results suggest that in the case of high p-ERK tumors, the abundance of TMEM119⁺ cells correspond to microglia^{34,36,37}. Moreover, the expression of MHC II in tumor infiltrating myeloid cells was more robust for TMEM119⁺ cells rather than CD163⁺ cells in high p-ERK tumors. Microglial cells have low levels of MHC II expression in homeostatic states^{69,70}. However, HLA-DR is upregulated in human glioma-infiltrating microglia compared to non-cancerous controls³⁷. On the other hand, it has been suggested that impaired MHC class II expression by glioma-infiltrating microglia leads to evasion from CD4⁺ T cells^{71,72}, which could explain the lack of response to PD-1 blockade in some GBM patients. Whereas previous studies have demonstrated the ability of human and murine microglia to impair tumor growth^{43,73-75}, their exact function in the context of immune checkpoint inhibition remains ill-defined. More so, single-cell technologies are increasingly dissecting with greater detail the diversity of myeloid cells in gliomas³⁴, so it will be interesting to see the impact of immune checkpoint blockade in the proportions and functionality of these immune cell populations. Considering that the tumoral immune composition varies between GBM patients^{2,39,40}, it is possible that a heterogenous clinical response to PD-1 blockade could derive from phenotypic differences of tumor-associated myeloid cells that are modulated by interchangeable cellular states recently described for glioma cells³.

We did not find an association between p-ERK and T cell infiltration (data not shown). This is not surprising as we studied immunotherapy-naïve specimens; T cell sequestration in the bone marrow and scant tumor-infiltrating effector T cells are characteristic of GBM patients⁹, and T cell infiltration remains modest at best in studies employing neoadjuvant PD-1 blockade¹⁶. In other solid tumors, neoadjuvant PD-1 blockade induced an expansion of peripheral T cell clones that were able to infiltrate tumors between 2 and 4 weeks after initiation of immunotherapy⁷⁶. In our cohort, we noticed an increase in CD4⁺ and CD8⁺ T cells in the CSF and the brain tissue in a high p-ERK GBM patient months after being treated with continuous PD-1 blockade (FIG. 1b). Though we acknowledge this is a description of a single case, it is possible that longer treatment periods with the anti-PD-1 antibody may induce a more robust infiltration and T cell effector activity. Due to the interval (2-3 weeks) between neoadjuvant PD-1 immunotherapy and tissue acquisition^{7,16}, current studies in GBM may not have captured this phenomenon.

One of the limitations of the current study is that it does not establish causality. Therefore, the mechanism underlying for why p-ERK is a predictor of PD-1 blockade response remains to be determined. Although we found OS differences between high and low p-ERK GBM patients in the discovery and validation cohorts, we acknowledge that another limitation of our study is the number of patients analyzed, and the retrospective design of the study. Therefore, prospective validation of this biomarker on a trial evaluating anti-PD-1 therapy is important. Additionally, given the emerging evidence of potential therapeutic benefit derived from neoadjuvant PD-1 blockade in GBM^{7,16,46}, another important unsolved question is whether p-ERK1/2 remains predictive of response in specimens obtained following initiation with this immunotherapy.

In summary, our results suggest that phosphorylation of ERK1/2 is indicative of response to adjuvant PD-1 blockade in recurrent GBM patients, even in the absence of activating mutations of BRAF and PTPN11 that promote MAPK pathway signaling. Thus, p-ERK1/2 staining might identify the majority of GBM patients that respond to this therapy. This may offer an opportunity to apply immunotherapy with a personalized approach for GBM, providing therapeutic benefit for a subset of patients while avoiding futile treatments for others.

Methods

Study design and patient selection. This is a retrospective cohort clinical study followed by a validation cohort of a prospective clinical trial that evaluates the association of p-ERK as biomarker for response to anti-PD-1 therapy in recurrent GBM patients. As part of the discovery cohort, a control group that did not receive PD-1 blockade was included for survival comparisons with the group that received the immunotherapy. For the discovery cohort, patients were at least 18 years old who were diagnosed with recurrent GBM and treated with either pembrolizumab or nivolumab or without immunotherapy from two institutions: Northwestern University (n=40) and Columbia University (n=22). Institutional review board approval was acquired from each institution. The study was conducted in accordance with the institutional ethical regulations and the Declaration of Helsinki principles. The clinical and molecular characteristics of the patients are provided in FIG. 20. Patients with tumor specimens' requirements for satisfactory neuropathological assessment were included for further analysis. Inclusion and Exclusion criteria are the following:

| Inclusion criteria | Exclusion criteria |
|---|---|
| Diagnosis of histologically confirmed | Patients that did not |
| recurrent glioblastoma | have adequate tumor tissue |
| Age ≥18 years old | for immunohistochemistry |
| Treatment with a PD-1 inhibitor | assessment at recurrence |
| (nivolumab or pembrolizumab) following | Patients that did not |
| surgery for resection of recurrent GBM | have clinical data |
| Patients must have received first line | available for analysis |
| treatment with temozolomide and | |
| radiotherapy | |

For the validation cohort, we analyzed the tumor samples of recurrent GBM patients (n=13) from Cloughesy T et al. (NCT02852655)¹⁶. We studied the adjuvant arm of this prospective controlled clinical trial. As mentioned in the original publication, patients received pembrolizumab 200 mg by intravenous infusion every 3 weeks after recovery from surgery until either tumor progression or an adverse event requiring study drug discontinuation. Clinical data is accessible in the referenced article and in FIG. 21.

Survival analysis. The primary endpoint in the discovery and validation cohorts is overall survival (OS). Secondary endpoints included OS rate at 12 months and progression-free survival (PFS). OS is defined as the time of initiation of anti-PD-1 therapy to the date of death from any cause for the immunotherapy cohort. For the no immunotherapy cohort, OS is defined as the time of recurrence to the date of death from any cause. OS was censored for GBM patients who were alive at the time of the cutoff date for the discovery and validation cohorts. In addition, clinical and molecular variables influencing survival were explored as part of a Cox proportional hazard model, including age, Karnofsky performance score (KPS), IDH mutational status, MGMT methylation, steroid dose at the time of immunotherapy, size of residual tumor determined by MM measurement, Ki67, and concurrent treatments. Partition of the groups into high and low p-ERK tumors was determined by using the median of the values derived from the software-based quantification of p-ERK cell density of all the GBM patients in the discovery cohort (anti-PD-1 therapy group and No immunotherapy cohort). The same value used to define high and low p-ERK tumors in the discovery cohort was applied to the validation cohort to define the same groups. Additionally, to evaluate the ability of p-ERK as a biomarker to select patients that could benefit from treatment with PD-1 blockade in the clinical setting, a neuropathologist scored the tumor samples from 0 to 3, for which specimens were defined as high (2 or 3) and low (0 or 1) p-ERK tumors. For both methods of partitioning into high and low p-ERK groups, software-based quantification of p-ERK cell density and neuropathological assessment of p-ERK-expressing tumor regions, we employed two statistical methods for survival analysis: 1) two-sided log-rank test; 2) Wald test resulting from univariable and multivariable Cox proportional hazard models with the quantification and the score of p-ERK as a variable. Multivariable models looked at the association between p-ERK and survival after adjusting for variables of interest as additive effects. The proportional hazards assumption was checked through the assessment of scaled Schoenfeld residuals. Error bars on forest plots indicate 95% confidence intervals for the hazard ratios. FIG. 19.

To calculate the mean AUC values in the discovery and validation cohort, we employed the R package pROC⁷⁷ in which we evaluated the ability of p-ERK cells/mm² to predict survival at 12 months after initiation of anti-PD-1 therapy in the discovery and validation cohort. To further explore the interaction between p-ERK and immunotherapy with respect to OS, cut-point optimization using conditional inference trees with the R package partykit^{78,79} (v.1.2-9) was performed for exploratory purposes. In optimizing the cut-point value for p-ERK, we used the PD-1 blockade treatment arm to avoid confounding due to differences in OS between arms (PD-1 blockade and No immunotherapy).

GBM samples. Tumor samples from participating institutions used for analysis for the discovery cohort (Northwestern University and Columbia University) were collected by the dedicated Brain Tumor Bank staff and clinical pathology cores that have standard operating procedures (SOPs) in which tissue is fixed and catalogued in a timely fashion. Slides were cut from blocks shortly prior to staining, so no unstained slides were stored for long periods.

Determination of the integrity of p-ERK from FFPE embedded tumors. Based on the availability of tissue blocks from the Nervous System Tumor Bank, we obtained tissue scrolls from a representative set of 7 low and 5 high p-ERK tumors from the same FFPE GBM tissues used for our survival analyses (n=12). Next, we extracted phosphoproteins and total proteins using the QProteome FFPE Tissue Kit following the manufacturer's protocol (Qiagen, cat. 37623). We performed western blot against p-ERK1/2 using the same antibody used to perform IHC in FFPE GBM tissues and 2 GBM cell lines representing positive (AM38, a BRAF mutant cell line) and negative (U251MG) controls. To assess the integrity of an additional phosphorylated protein in these tumors, we evaluated the phospho-AKT (4060, clone: D9E, Cell Signaling Technology, dilution 1:1000), ERK1 (13-8600, Thermo Fisher Scientific, dilution 1:1000), ERK2 (9108, Cell Signaling Technology, dilution 1:1000), and β-actin (3700, Cell Signaling Technology, dilution 1:1000).

Peptide competition assay. The phospho-p44/42 (ERK1/2) antibody (4370S, Cell Signaling Technology) was incubated overnight at 4° C. with a specific blocking peptide in a 1:10 dilution that saturated the p-ERK antibody (phospho-p44/42 MAPK (ERK1/2) blocking peptide, catalog #1150, Cell Signaling Technology). As controls for the blocking peptide, this was also incubated with the antibodies detecting p-EGFR and p-AKT. Western blot was performed in GBM samples evaluating p-ERK (4370, Cell Signaling Technology, dilution 1:1000), ERK1/2 (4695, Cell Signaling Technology, dilution 1:1000), p-EGFR (3777, clone: D7A5, Cell Signaling Technology, dilution 1:1000), EGFR (4267, clone: D38B1, Cell Signaling Technology, dilution 1:1000), p-AKT (4060, clone: D9E, Cell Signaling Technology, dilution 1:1000), AKT (4691, clone: C67E7, Cell Signaling Technology, dilution 1:1000), and β-actin (3700, Cell Signaling Technology, dilution 1:1000). Additionally, the specificity of the p-ERK1/2 antibody was evaluated by IHC in the same set of samples used to perform the peptide competition assay by western blot.

Western Blot. Western blotting was performed using conventional protocols. In brief, cells from fresh tumor specimens were lysed with M-PER buffer (Thermo Fisher Scientific) which contained protease and phosphatase inhibitors (Thermo Fisher Scientific). After quantification of proteins acquired from either FFPE GBM tissues or fresh tumor specimens, these were loaded in a 4-20% Mini-PROTEAN TGX Stain-Free protein gel (Biorad) for electrophoresis. Then, blots were blocked for 1 hour at room temperature with 5% milk in 10 mM Tris-HCl pH 7.5, 150 mM NaCl containing 0.1% Tween 20 (TBST) and incubated at room temperature for 1 hour with the corresponding primary antibodies. Blots were washed after incubation with the primary antibodies and then incubated with an anti-rabbit IgG HRP-linked antibody (7074, Cell Signaling Technology, dilution 1:2000) for 1 hour at room temperature. Finally, antigen-antibody reaction was detected using Clarity Western ECL substrate kit (catalog #1705061, Biorad) following manufacturer's protocols.

Evaluation of the effect of ischemic time on p-ERK stability. 3 human tumor specimens were obtained during neurosurgery, and immediately divided into similar size portions, which then were subjected to different ischemic times (0 hr., 0.5 hr., 1 hr., and 2 hrs.) before fixation. Next, samples were processed and embedded in paraffin blocks that were cut to get tissue sections. IHC staining was performed in these sections with the same dilution used to stain all GBM samples in the study. Stained slides were scanned to perform image analysis of endothelial cells. Depending on the number of vessels in each tumor, 5 to 24 ROIs each corresponding to one endothelial cell were acquired for tumor samples subjected to different ischemic times and from tumor of the discovery cohort. Next, the intensity of endothelial cells fixed immediately after resection (0 hr. of ischemic time) was used as the reference to compare the intensity of all the tumor samples.

Immunohistochemistry staining. IHC and H&E staining was performed using standard immunoperoxidase staining on formalin-fixed paraffin-embedded tissue sections of 5 μm thick from resected recurrent tumors. Sections were stained with (mouse) anti-human monoclonal antibodies against phospho-p44/42 (ERK1/2) (4370S, Cell Signaling Technology, dilution 1:500), mouse anti-Iba1 (Abcam, dilution 1:1000) mouse anti-CD3 (DakoCytomation, dilution 1:200). The procedure was performed on a DAKO Autostainer Link 48 slide stainer (Agilent Technologies). Paraffin sections were deparaffinized with xylene in the stainer and then underwent heat-mediated antigen retrieval with sodium citrate buffer. Sections were counterstained with hematoxylin, dehydrated, and mounted with coverslips. The slides were scanned and digitalized with the Hamamatsu K.K. Nanozoomer 2.0 HT and were visualized with the NDP.view2 Viewing software. A board-certified neuropathologist evaluated the staining digitally to ensure the appropriate quality of the tumor tissue.

IHC image analysis. For the quantification of the IHC images for p-ERK, a neuropathologist outlined the tumoral regions on each sample in a blinded fashion regarding treatments, survival outcomes, and other clinical characteristics. We employed the HistoQuest version 6.0 software (TissueGnostics) for the semiautomatic quantification of p-ERK cell density. We employed a nuclear segmentation method, a ring mask with interior radius of −0.45 μm and exterior radius of 0.91 μm, and a cytoplasmic cell mask. For each cell, the nucleus was segmented and a cytoplasmic region surrounding the nucleus was defined. To identify positive nuclei, we adjusted the software parameters in order to detect hematoxylin alone (color RGB: 30, 45, 84) and DAB chromogen (color RGB: 94, 48, 14) combined with hematoxylin. To consider a cell positive for p-ERK, stained cells had to exceed the mean intensity threshold of 150 in the cytoplasmic compartment after the machine identified a nucleus. p-ERK⁺ cell density was defined as the number of p-ERK⁺ cells in a given area (mm²). For the quantification of Iba1, we employed a nuclear segmentation method with cell mask segmentation without using a ring mask. Then, we taught the machine which was the positive signal derived from the DAB chromogen in the color separation parameters (color RGB: 81, 41, 16). Positive Iba1 signal was the result of a mixture between the DAB chromogen and hematoxylin (color RGB: 102, 109, 128). A mean intensity threshold of 130 was set to define a cell positive for Iba1. Similar to p-ERK cell quantification, we analyzed the tumor regions that the neuropathologist delineated. p-ERK and Iba1 cell density was defined as the number of p-ERK⁺ or Iba1⁺ cells in a given area (mm²). For the tumor samples that had 2 or more spatially separated tumor regions, these tissues were quantified, and the resulting values were averaged to provide a single value for a particular tumor sample.

Tumor sample processing for flow cytometry data acquisition and analysis. GBM tumor samples were collected and kept in cold phosphate buffer solution (PBS) before processing the samples for FACS analyses. Tumor samples were gently dissociated using 70 μm cell strainers (Fisher Scientific 352350) in cold FACS buffer (PBS+2 mM EDTA+BSA 2%) and washed two times in a large volume of FACS buffer (50 mL). Cells were incubated for 5 minutes with human Fc block (BD, 564219) on ice and then incubated with antibodies at 4° C. for 30 mins. Cells were washed, and 7AAD (BD, 559925) was added (1:100 dilution) before FACS acquisition.

Blood samples were collected on heparin tubes and mix at a 1:1 ratio with DMEM medium at room temperature to perform lymphocyte (BD, NC9587917) PBMC isolation. PBMC were washed in FACS buffer and stained similarly to the tumor samples. Antibodies used were APC anti-CD45 (Biolegend, 304012), PE-Cy7 anti-CD8a (eBiosciences, 25-0087-42), APC-eFluor 780 anti-CD4 (eBiosciences, 47-0049-42). Data were acquired on BD Fortessa LSRII and analyzed by first gating live cells, then CD45⁺ cells, and finally CD4⁺ cells in the X axis and CD8⁺ in the Y axis of the scatter plot. All the analyses were conducted using FlowJo v. 10.6.2.

Multiplex immunofluorescence staining. Sections of 5-μm thickness were obtained from FFPE embedded tumor tissue. Deparaffinization of the slides was done with xylene, and then rehydrated in histological grade ethanol and fixed with 3% hydrogen peroxide in methanol before antigen retrieval using pH6 citrate buffer or pH9 EDTA buffer. DAB staining was first performed to determine the optimal concentrations of each antibody. For the first panel, the primary antibodies used were: p-ERK (catalog 4370, clone D13.14.4E, Cell Signaling Technology, 1:1600), CD163 (catalog ab213612, clone EPR19518, Abcam, 1:600), MHC II (catalog ab180779, Abcam, 1:400), TMEM119 (catalog HPA051870, Sigma-Aldrich, 1:250), SOX2 (catalog ab92494, clone EPR3131, Abcam, 1:5000). Tyramide signal amplification (TSA) visualization was done using the Opal 7-color IHC Kit (NEL821001KT, Akoya Biosciences) that includes 6 reactive fluorophores of which we used Opal 520 (1:100), Opal 540 (1:200), Opal 570 (1:800), Opal 620 (1:150), Opal 690 (1:100) and DAPI. After optimization of each antibody, the multiplex staining for 7 colors was performed with an antigen retrieval step, protein blocking, epitope labeling, and signal amplification between each cycle. Finally, we used Spectral DAPI (Akoya Biosciences) to counterstain the slides and were mounted with long lasting aqueous-based mounting medium.

For the second panel, the primary antibodies used were the following: p-ERK (clone D13.14.4E, Cell Signaling Technology, 1:1600), GFAP (catalog ab68428, clone EPR1034Y, Abcam, 1:300), CD163 (catalog ab213612, clone EPR19518, Abcam, 1:100). Tyramide signal amplification (TSA) visualization was done using the Opal 7-color IHC Kit (NEL821001KT, Akoya Biosciences) that includes 6 reactive fluorophores Opal 480 (1:350), Opal 520 (1:200), Opal 570 (1:50), Opal 620 (1:250), Opal 690 (1:600), Opal 780 (1:300), and DAPI. After optimization of each antibody, the multiplex staining for 7 colors was performed with an antigen retrieval step, protein blocking, epitope labeling, and signal amplification between each cycle. Finally, we used Spectral DAPI (Akoya Biosciences) to counterstain the slides and were mounted with long lasting aqueous-based mounting medium.

Imaging and analysis of multispectral images. For the first panel, multispectral imaging (MSI) was performed using the Vectra 3 Automated Quantitative Pathology Imaging System from Akoya Biosciences. Briefly, whole slide images were acquired after autoadjusting focus and signal intensity. Then, MSI was acquired in the tumor regions delineated by the neuropathologist at 20× of original magnification. Then, we created a spectral library for all fluorophores to subject acquired multispectral images to spectral unmixing to visualize the signal of each marker (SOX2, p-ERK, TMEM119, CD163, MHC II, DAPI) in inForm Tissue Finder software (inForm 2.4.9, Akoya Biosciences). Nuclear-based cell segmentation using DAPI was performed as well as phenotyping of the cell markers on inForm.

For the second panel, the stained slides were scanned using Vectra Polaris (Akoya Biosciences). Images were visualized and analyzed using Phenochart (v1.0.12) to select multiple regions of interest (ROIs) encompassing the tumoral regions delineated by the neuropathologist to maintain consistency with the IHC quantification analysis. The selected ROIs were uploaded to inForm Tissue Finder software (inForm 2.4.9, Akoya Biosciences) to subject the images to spectral unmixing that allows the evaluation and separation of weak and overlapping signals from the background autofluorescence for each single marker of each antibody. Then, after adjusting the parameters to identify nucleus of the analyzed cells, cell segmentation was performed to determine the nuclear and cytoplasmic compartments on each cell. For the two panels, we used a machine-learning algorithm within inForm in which cells were automatically assigned to a specific phenotype (SOX2⁺, TMEM119⁺, CD163⁺, p-ERK⁺, MHC II⁺) (CD163⁺, GFAP⁺, p-ERK⁺, CD163⁻, GFAP⁻, p-ERK⁻). The processing and analysis of images from all tumor samples were exported to cell segmentation tables. Exported files from inForm were processed in R using R packages Phenoptr and PhenoptrReports to merge and create consolidated single files for each tumor sample. Consolidated files had double cell phenotypes as outputs that we employed for further quantification and spatial analyses using the Phenoptr R addin.

Cell quantification and spatial analysis of multiplex immunofluorescence images. The consolidated files obtained using Phenoptr were analyzed to quantify the density of SOX2⁺ p-ERK⁺, SOX2⁺ p-ERK⁻, TMEM119⁺, CD163⁺, TMEM119⁺ MHC and CD163⁺ MHC II⁺ For the spatial analysis, mean cell counts within a specified radius of 15 μm from SOX2⁺ p-ERK⁺ cells to TMEM119⁺ were calculated using Phenoptr as an R addin. Then, mean distance between the nearest neighbors were calculated from myeloid cells (TMEM119⁺ and CD163+ cells) to SOX2 p-ERK⁺ and SOX2 p-ERK⁻ cells. The spatial map viewer addin within R allowed the visualization of nearest cell neighbor between selected phenotypes in a single field of a high and low p-ERK tumor.

For the second panel, we followed the same analysis of consolidated files in which we identified GFAP⁺, CD163+, GFAP⁺ p-ERK⁺ cells, CD163⁺ p-ERK⁺ cells, and GFAP⁻ CD163⁻ p-ERK⁺ cells. For the spatial analysis, mean cell counts within a specified radius of 15 μm from GFAP⁺ p-ERK⁺ cells to CD163⁺ cells were calculated using Phenoptr. Additionally, mean distances between the nearest neighbor were calculated from CD163⁺ cells to GFAP⁺ p-ERK⁺ cells and from CD163⁺ cells to GFAP⁺ p-ERK⁻ cells for each recurrent tumor sample. For the visualization of distances between CD163⁺ cells and GFAP⁺ p-ERK⁺ cells and GFAP⁺ p-ERK-cells, the spatial map viewer within R was used selecting the desired phenotypes in particular fields. Cartoons were created using Adobe Illustrator version 22.1.0.

scRNA-seq processing. Single-cell RNA-seq data was obtained from 10 GBM cases previously published by Yuan, et al.³⁵ All datasets were first filtered to remove genes coding for mitochondrial and ribosomal proteins. Count matrices for each case were then merged together keeping the union of genes, with genes with zero total counts being discarded. Raw counts were then normalized to log 2(1+TPK), as described in Yuan, et al.³⁵. Z-scores for gene expression were calculated based on these normalized counts. For visualization: first Principal Components Analysis (PCA) was applied to reduce the total dimensionality to 5% of the number of genes, then Uniform Manifold Approximation and Projection (UMAP)⁸⁰ with default parameters to non-linearly reduce that into a two-dimensional embedding. Agreeing with the previous analysis from Yuan, et al.³⁵, four cell types were readily identified using the standard markers of CD14, VWF, PDGFRB, and SOX2 for myeloid, endothelial, pericytes, and tumor cells, respectively.

Statistical analysis. GraphPad Prism v6.0c and 8, Python 3.6, R v. 4.0.2, and Microsoft Excel v16.33 were used for statistical analyses. Numerical data was reported as mean sd. For the analysis requiring two-sided nonparametric calculations, Mann-Whitney U-test was used for non-paired observations. Pearson's r was used to correlate raw data values of the indicated variables depicting linear relationships. One-way ANOVA was used for multiple comparisons and P values were adjusted using the Tukey's test for multiple comparisons. For comparisons, adjusted P value threshold of 0.05 was considered statistically significant.

Data and code availability. Data and custom codes will be available upon reasonable request. Raw sequencing data for the samples utilized for our scRNA-seq analysis can be encountered on GEO under the series GSE103224 and GSE141383.

### REFERENCES

1. Stupp, R., et al. Effect of Tumor-Treating Fields Plus Maintenance Temozolomide vs Maintenance Temozolomide Alone on Survival in Patients With Glioblastoma: A Randomized Clinical Trial. JAMA 318, 2306-2316 (2017).
2. Wang, Q., et al. Tumor Evolution of Glioma-Intrinsic Gene Expression Subtypes Associates with Immunological Changes in the Microenvironment. Cancer Cell 32, 42-56.e46 (2017).
3. Neftel, C., et al. An Integrative Model of Cellular States, Plasticity, and Genetics for Glioblastoma. Cell 178, 835-849.e821 (2019).
4. Gong, J., Chehrazi-Raffle, A., Reddi, S. & Salgia, R. Development of PD-1 and PD-L1 inhibitors as a form of cancer immunotherapy: a comprehensive review of registration trials and future considerations. J Immunother Cancer 6, 8 (2018).
5. Reardon, D. A., et al. Effect of Nivolumab vs Bevacizumab in Patients With Recurrent Glioblastoma: The CheckMate 143 Phase 3 Randomized Clinical Trial. JAMA Oncol (2020).
6. Nayak, L., et al. Randomized phase II and biomarker study of pembrolizumab plus bevacizumab versus pembrolizumab alone for recurrent glioblastoma patients. Clin Cancer Res (2020).
7. de Groot, J., et al. Window-of-opportunity clinical trial of pembrolizumab in patients with recurrent glioblastoma reveals predominance of immune-suppressive macrophages. Neuro Oncol 22, 539-549 (2020).
8. Arrieta, V. A., et al. The possibility of cancer immune editing in gliomas. A critical review. Oncoimmunology 7, e1445458 (2018).
9. Chongsathidkiet, P., et al. Sequestration of T cells in bone marrow in the setting of glioblastoma and other intracranial tumors. Nat Med 24, 1459-1468 (2018).
10. Jackson, C. M., Choi, J. & Lim, M. Mechanisms of immunotherapy resistance: lessons from glioblastoma. Nat Immunol 20, 1100-1109 (2019).
11. Arrieta, V. A., et al. Can patient selection and neoadjuvant administration resuscitate PD-1 inhibitors for glioblastoma? J Neurosurg, 1-6 (2019).
12. Woroniecka, K., et al. T-Cell Exhaustion Signatures Vary with Tumor Type and Are Severe in Glioblastoma. Clin Cancer Res 24, 4175-4186 (2018).
13. Lukas, R. V., Wainwright, D. A., Horbinski, C. M., Iwamoto, F. M. & Sonabend, A. M. Immunotherapy Against Gliomas: is the Breakthrough Near? Drugs 79, 1839-1848 (2019).
14. Zhao, J., et al. Immune and genomic correlates of response to anti-PD-1 immunotherapy in glioblastoma. Nat Med 25, 462-469 (2019).
15. Lukas, R. V., et al. Clinical activity and safety of atezolizumab in patients with recurrent glioblastoma. J Neurooncol 140, 317-328 (2018).
16. Cloughesy, T. F., et al. Neoadjuvant anti-PD-1 immunotherapy promotes a survival benefit with intratumoral and systemic immune responses in recurrent glioblastoma. Nat Med 25, 477-486 (2019).
17. Davies, H., et al. Mutations of the BRAF gene in human cancer. Nature 417, 949-954 (2002).
18. Dhillon, A. S., Hagan, S., Rath, 0. & Kolch, W. MAP kinase signalling pathways in cancer. Oncogene 26, 3279-3290 (2007).
19. Ruess, D. A., et al. Mutant KRAS-driven cancers depend on PTPN11/SHP2 phosphatase. Nat Med 24, 954-960 (2018).
20. Milde-Langosch, K., et al. Expression and prognostic relevance of activated extracellular-regulated kinases (ERK1/2) in breast cancer. Br J Cancer 92, 2206-2215 (2005).
21. Vicent, S., et al. ERK1/2 is activated in non-small-cell lung cancer and associated with advanced tumours. Br J Cancer 90, 1047-1052 (2004).
22. Svensson, S., et al. ERK phosphorylation is linked to VEGFR2 expression and Ets-2 phosphorylation in breast cancer and is associated with tamoxifen treatment resistance and small tumours with good prognosis. Oncogene 24, 4370-4379 (2005).
23. Wen, P. Y., et al. Updated response assessment criteria for high-grade gliomas: response assessment in neuro-oncology working group. J Clin Oncol 28, 1963-1972 (2010).
24. Ranjan, S., et al. Clinical decision making in the era of immunotherapy for high grade-glioma: report of four cases. BMC Cancer 18, 239 (2018).
25. Roth, P., Valavanis, A. & Weller, M. Long-term control and partial remission after initial pseudoprogression of glioblastoma by anti-PD-1 treatment with nivolumab. Neuro Oncol 19, 454-456 (2017).
26. Okada, H., et al. Immunotherapy response assessment in neuro-oncology: a report of the RANO working group. Lancet Oncol 16, e534-e542 (2015).
27. Gramatzki, D., et al. Limited role for extended maintenance temozolomide for newly diagnosed glioblastoma. Neurology 88, 1422-1430 (2017).
28. Yan, H., et al. IDH1 and IDH2 mutations in gliomas. N Engl J Med 360, 765-773 (2009).
29. Hegi, M. E., et al. MGMT gene silencing and benefit from temozolomide in glioblastoma. N Engl J Med 352, 997-1003 (2005).
30. Vassilakopoulou, M., et al. Preanalytical variables and phosphoepitope expression in FFPE tissue: quantitative epitope assessment after variable cold ischemic time. Lab Invest 95, 334-341 (2015).
31. Holzer, T. R., et al. Ischemic time impacts biological integrity of phospho-proteins in PI3K/Akt, Erk/MAPK, and p38 MAPK signaling networks. Anticancer Res 31, 2073-2081 (2011).
32. Narasimhan, P., Liu, J., Song, Y. S., Massengale, J. L. & Chan, P. H. VEGF Stimulates the ERK 1/2 signaling pathway and apoptosis in cerebral endothelial cells after ischemic conditions. Stroke 40, 1467-1473 (2009).
33. Nagasawa-Masuda, A. & Terai, K. ERK activation in endothelial cells is a novel marker during neovasculogenesis. Genes Cells 21, 1164-1175 (2016).
34. Pombo Antunes, A. R., et al. Single-cell profiling of myeloid cells in glioblastoma across species and disease stage reveals macrophage competition and specialization. Nat Neurosci 24, 595-610 (2021).
35. Yuan, J., et al. Single-cell transcriptome analysis of lineage diversity in high-grade glioma. Genome Med 10, 57 (2018).
36. Bennett, M. L., et al. New tools for studying microglia in the mouse and human CNS. Proc Natl Acad Sci USA 113, E1738-1746 (2016).
37. Sankowski, R., et al. Mapping microglia states in the human brain through the integration of high-dimensional techniques. Nat Neurosci 22, 2098-2110 (2019).
38. Moyes, K. W., et al. Effects of tumor grade and dexamethasone on myeloid cells in patients with glioma. Oncoimmunology 7, e1507668 (2018).
39. Friebel, E., et al. Single-Cell Mapping of Human Brain Cancer Reveals Tumor-Specific Instruction of Tissue-Invading Leukocytes. Cell 181, 1626-1642.e1620 (2020).
40. Klemm, F., et al. Interrogation of the Microenvironmental Landscape in Brain Tumors Reveals Disease-Specific Alterations of Immune Cells. Cell 181, 1643-1660.e1617 (2020).
41. Saha, D., Martuza, R. L. & Rabkin, S. D. Macrophage Polarization Contributes to Glioblastoma Eradication by Combination Immunovirotherapy and Immune Checkpoint Blockade. Cancer Cell 32, 253-267.e255 (2017).
42. Goswami, S., et al. Immune profiling of human tumors identifies CD73 as a combinatorial target in glioblastoma. Nat Med 26, 39-46 (2020).
43. Rao, G., et al. Anti-PD-1 Induces M1 Polarization in the Glioma Microenvironment and Exerts Therapeutic Efficacy in the Absence of CD8 Cytotoxic T Cells. Clin Cancer Res 26, 4699-4712 (2020).
44. Keren, L., et al. A Structured Tumor-Immune Microenvironment in Triple Negative Breast Cancer Revealed by Multiplexed Ion Beam Imaging. Cell 174, 1373-1387.e1319 (2018).
45. Subramanian, A., et al. Gene set enrichment analysis: a knowledge-based approach for interpreting genome-wide expression profiles. Proc Natl Acad Sci USA 102, 15545-15550 (2005).
46. Schalper, K. A., et al. Neoadjuvant nivolumab modifies the tumor immune microenvironment in resectable glioblastoma. Nat Med 25, 470-476 (2019).
47. Motzer, R. J., et al. Nivolumab versus Everolimus in Advanced Renal-Cell Carcinoma. N Engl J Med 373, 1803-1813 (2015).
48. Le, D. T., et al. Mismatch repair deficiency predicts response of solid tumors to PD-1 blockade. Science 357, 409-413 (2017).
49. Hellmann, M. D., et al. Nivolumab plus Ipilimumab in Lung Cancer with a High Tumor Mutational Burden. N Engl J Med 378, 2093-2104 (2018).
50. Cristescu, R., et al. Pan-tumor genomic biomarkers for PD-1 checkpoint blockade-based immunotherapy. Science 362(2018).
51. Johanns, T. M., et al. Immunogenomics of Hypermutated Glioblastoma: A Patient with Germline POLE Deficiency Treated with Checkpoint Blockade Immunotherapy. Cancer Discov 6, 1230-1236 (2016).
52. Bouffet, E., et al. Immune Checkpoint Inhibition for Hypermutant Glioblastoma Multiforme Resulting From Germline Biallelic Mismatch Repair Deficiency. J Clin Oncol 34, 2206-2211 (2016).
53. Ahmad, H., Fadul, C. E., Schiff, D. & Purow, B. Checkpoint inhibitor failure in hypermutated and mismatch repair-mutated recurrent high-grade gliomas. Neurooncol Pract 6, 424-427 (2019).
54. Samstein, R. M., et al. Tumor mutational load predicts survival after immunotherapy across multiple cancer types. Nat Genet 51, 202-206 (2019).
55. Touat, M., et al. Mechanisms and therapeutic implications of hypermutation in gliomas Nature 580, 517-523 (2020).
56. McGranahan, N., et al. Clonal neoantigens elicit T cell immunoreactivity and sensitivity to immune checkpoint blockade. Science 351, 1463-1469 (2016).
57. Wick, W., et al. Lomustine and Bevacizumab in Progressive Glioblastoma. N Engl J Med 377, 1954-1963 (2017).
58. Stupp, R., et al. NovoTTF-100A versus physician's choice chemotherapy in recurrent glioblastoma: a randomised phase III trial of a novel treatment modality. Eur J Cancer 48, 2192-2202 (2012).
59. Azoulay, M., et al. Benefit of re-operation and salvage therapies for recurrent glioblastoma multiforme: results from a single institution. J Neurooncol 132, 419-426 (2017).
60. Ryken, T. C., Kalkanis, S. N., Buatti, J. M., Olson, J. J. & Committee, A. C. J. G. The role of cytoreductive surgery in the management of progressive glioblastoma: a systematic review and evidence-based clinical practice guideline. J Neurooncol 118, 479-488 (2014).
61. Robert, K. J., et al. CD8⁺ T-cell-mediated immunoediting influences genomic evolution and immune evasion in murine gliomas. Clin Cancer Res 26, 4390-4401 (2020).
62. Alban, T. J., et al. Global immune fingerprinting in glioblastoma patient peripheral blood reveals immune-suppression signatures associated with prognosis. JCI Insight 3(2018).
63. Chen, P., et al. Symbiotic Macrophage-Glioma Cell Interactions Reveal Synthetic Lethality in PTEN-Null Glioma. Cancer Cell 35, 868-884.e866 (2019).
64. Hutter, G., et al. Microglia are effector cells of CD47-SIRPα antiphagocytic axis disruption against glioblastoma. Proc Natl Acad Sci USA 116, 997-1006 (2019).
65. Strauss, L., et al. Targeted deletion of PD-1 in myeloid cells induces antitumor immunity. Sci Immunol 5(2020).
66. Gromeier, M., et al. Very low mutation burden is a feature of inflamed recurrent glioblastomas responsive to cancer immunotherapy. Nat Commun 12, 352 (2021).
67. Liu, D., et al. Integrative molecular and clinical modeling of clinical outcomes to PD1 blockade in patients with metastatic melanoma. Nat Med 25, 1916-1927 (2019).
68. Alspach, E., et al. MHC-II neoantigens shape tumour immunity and response to immunotherapy. Nature 574, 696-701 (2019).
69. Mrdjen, D., et al. High-Dimensional Single-Cell Mapping of Central Nervous System Immune Cells Reveals Distinct Myeloid Subsets in Health, Aging, and Disease. Immunity 48, 380-395.e386 (2018).
70. Mundt, S., et al. Conventional DCs sample and present myelin antigens in the healthy CNS and allow parenchymal T cell entry to initiate neuroinflammation. Sci Immunol 4(2019).
71. Qian, J., et al. TLR2 Promotes Glioma Immune Evasion by Downregulating MHC Class II Molecules in Microglia. Cancer Immunol Res 6, 1220-1233 (2018).
72. Schartner, J. M., et al. Impaired capacity for upregulation of MHC class II in tumor-associated microglia. Glia 51, 279-285 (2005).
73. Sarkar, S., et al. Therapeutic activation of macrophages and microglia to suppress brain tumor-initiating cells. Nat Neurosci 17, 46-55 (2014).
74. Mora, R., et al. TNF-alpha- and TRAIL-resistant glioma cells undergo autophagy-dependent cell death induced by activated microglia. Glia 57, 561-581 (2009).
75. Sarkar, S., et al. Microglia induces Gas1 expression in human brain tumor-initiating cells to reduce tumorigenecity. Sci Rep 8, 15286 (2018).
76. Zhang, J., et al. Compartmental Analysis of T-cell Clonal Dynamics as a Function of Pathologic Response to Neoadjuvant PD-1 Blockade in Resectable Non-Small Cell Lung Cancer. Clin Cancer Res 26, 1327-1337 (2020).
77. Robin, X., et al. pROC: an open-source package for R and S+ to analyze and compare ROC curves. BMC Bioinformatics 12, 77 (2011).
78. T, H., K, H. & A, Z. Unbiased Recursive Partitioning: A Conditional Inference Framework. Journal of Computational and Graphical Statistics 15(3), 651-674 (2006).
79. T, H. & A, Z. partykit: A Modular Toolkit for Recursive Partytioning in R. Journal of Machine Learning Research 16, 3905-3909 (2015).
80. McInnes, L., Healy, J., Saul, N. & Großberger, L. UMAP: Uniform Manifold Approximation and Projection. Journal of Open Source Software 3(2018).
81. Patel, A. P., et al. Single-cell RNA-seq highlights intratumoral heterogeneity in primary glioblastoma. Science 344, 1396-1401 (2014).
82. Liberzon, A., et al. Molecular signatures database (MSigDB) 3.0. Bioinformatics 27, 1739-1740 (2011).

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. A method for treating malignant glioma in a subject in need thereof, wherein the subject has a high, or an increased level of phosphorylated extracellular-signal-regulated kinase (p-ERK) compared to a reference level or a control subject, the method comprising: administering to the subject a programmed death protein 1 (PD-1) inhibitor, or a programmed death-ligand 1 (PD-L1) inhibitor.

2. The method of claim 1, wherein the malignant glioma is glioblastoma (GBM).

3. The method of claim 1, wherein the PD-1 inhibitor is administered with one or more additional therapeutic agents.

4. The method of claim 1, wherein the p-ERK level is determined in glioma tumor tissue.

5. The method of claim 1, wherein the PD-1 inhibitor comprises one or more of Nivolumab, Pembrolizumab, Cemiplimab, Spartalizumab (PDR001), Camrelizumab (SHR1210), Sintilimab (IBI308), Tislelizumab (BGB-A317), Toripalimab (JS 001), Dostarlimab (TSR-042, WBP-285), INCMGA00012 (MGA012), AMP-224, and AMP-514 (MED10680), Balstilimab (AGEN2034).

6. (canceled)

7. The method of claim 1, wherein the PD-L1 inhibitor comprises one or more of Atezolizumab, Avelumab, Durvalumab, KN035, CK-301, AUNP12, CA-170, and BMS-986189.

8. The method of claim 6, further comprising administering a PD-1 inhibitor.

9. The method of claim 1, wherein the subject does not have a mutation in the BRAF gene or the PTPN11 gene.

10. A method comprising:
determining the level of p-ERK in a malignant glioma tumor sample from a subject; and
if the p-ERK level is higher than the p-ERK level in a control sample or a reference p-ERK level, administering to the subject a PD-1 inhibitor, or administering to the subject a PD-L1 inhibitor.

11. The method of claim 10, wherein the malignant glioma comprises GBM.

12. The method of claim 11, wherein the GBM is recurrent.

13-15. (canceled)

16. A method of classifying a subject diagnosed with malignant glioma as a candidate for PD-1 inhibitor therapy or for PD-L1 inhibitor therapy, the method comprising:
determining the level of p-ERK in a glioma tumor sample from the subject;
comparing the determined p-ERK level with a control p-ERK level or a reference p-ERK level, wherein if the determined level is higher than the control level or the reference level, classifying the subject as a candidate for PD-1 inhibitor therapy or classifying the subject as a candidate for PD-L1 inhibitor therapy.

17. The method of claim 16, wherein the malignant glioma comprises GBM.

18. The method of claim 17, wherein the GBM is recurrent.

19-21. (canceled)


SEQUENCE LISTING (2 sequences)

SEQ ID NO: 1 - protein, 379 aa, Homo sapiens
MAAAAAQGGGGGEPRRTEGVGPGVPGEVEMVKGQPFDVGPRYTQLQYIGEGAYGMVSSAYDHVRKTRVAIKKISPFEHQTYCQRTLREIQILLRFRHENVIGIRDILRASTLEAMRDVYIVQDLMETDLYKLLKSQQLSNDHICYFLYQILRGLKYIHSANVLHRDLKPSNLLINTTCDLKICDFGLARIADPEHDHTGFLTEYVATRWYRAPEIMLNSKGYTKSIDIWSVGCILAEMLSNRPIFPGKHYLDQLNHILGILGSPSQEDLNCIINMKARNYLQSLPSKTKVAWAKLFPKSDSKALDLLDRMLTFNPNKRITVEEALAHPYLEQYYDPTDEPVAEEPFTFAMELDDLPKERLKELIFQETARFQPGVLEAP

SEQ ID NO: 2 - protein, 360 aa, Homo sapiens [sequence omitted]